(19) United States
(12) Patent Application Publication
(10) Pub. No.: US 2026/0306814 A1
(43) Pub. Date: Oct. 8, 2026

(54) PRECISION ADMINISTRATION OF ULTRA-LOADED INJECTABLE NANOCARRIERS

(71) Applicants: Massachusetts Institute of Technology, Cambridge, MA (US); The Brigham and Women's Hospital, Inc., Boston, MA (US)

(72) Inventors: Carlo Giovanni Traverso, Newton, MA (US); Jason Siu-Wei Li, Cambridge, MA (US); Seungho Lee, Cambridge, MA (US); Will Tritschler, Boston, MA (US); Nabil Shalabi, Lexington, MA (US); Yogesh Deshpande, Watertown, MA (US); Cooper Konz, Somerville, MA (US)

(73) Assignees: Massachusetts Institute of Technology, Cambridge, MA (US); The Brigham and Women's Hospital, Inc., Boston, MA (US)

(21) Appl. No.: 19/552,372

(22) Filed: Feb. 27, 2026

Related U.S. Application Data
(60) Provisional application No. 63/764,203, filed on Feb. 27, 2025.

(51) Int. Cl. A61M 37/00

(52) U.S. Cl. CPC A61M 37/0015; A61M 2037/0023; A61M 2205/21; A61M 2205/8281; A61M 2210/1053

(57) ABSTRACT

Self-righting articles, such as self-righting capsules for administration to a subject, are generally provided. In some embodiments, the self-righting article is configured to orient itself relative to a surface, e.g., along a tissue engaging surface. In some embodiments, the self-righting article has a particular shape and/or distribution of density (or mass) that enables the self-righting behavior of the article. In some embodiments, the self-righting article comprises a tissue interfacing component and/or a pharmaceutical agent. In some embodiments, the self-righting article is configured to release one or more tissue interfacing components.

### FIELD

The present invention generally relates to self-righting systems and related components such as loaded injectable nanocarriers, as well as components with relatively high loading of active pharmaceutical ingredients (API).

### BACKGROUND

The GI tract offers an incredible opportunity for diagnosing and treating patients. The development of smart dosage systems and articles to enable this has witnessed significant growth over the preceding decade. One of the most significant challenges in maximizing delivery and interaction with the mucosa is ensuring juxtaposition between an article and/or dosing system and the GI mucosa. Prior attempts at doing this have included the introduction of mucoadhesive as well as texturing of one side of a 2-sided system. Orally ingested drugs generally diffuse through the GI tract tissue walls in order to enter the blood stream. Typical ingested pills or articles release their cargo into the GI tract randomly and allow it to move via convection and diffusion to the tissue wall. However, many biologic drugs such as insulin cannot move through the liquid in the GI tract because they will be, for example, degraded by enzymes, even if housed in a solid formulation.

Additionally, many pharmaceutical drug formulations on the market require administration via in injection, including numerous vaccines, RNA, and peptides. Injections traditionally involve the use of a liquid formulation passing through a hollow needle and entering into the body intravenously or intramuscularly. However, these liquid formulations can cause the active pharmaceutical ingredient (API) to become unstable and thus may require refrigeration and/or increase the bulk of the dose significantly because of the required dilution.

Accordingly, improved systems, articles and methods are needed.

### SUMMARY

The present invention generally relates to self-righting articles.

In some embodiments, the self-righting article comprises (i) a tissue-engaging surface, (ii) one or more curved surfaces adjacent the tissue-engaging surface, (iii) a drug-containing portion, wherein the drug-containing portion occupies greater than or equal to 30 vol % of the self-righting article and/or the drug-containing portion comprises greater than or equal to 250 uL of drug, (iv) a tissue interfacing component, (v) an actuation mechanism configured to actuate horizontally (e.g., within 45 degrees of orthogonal) with respect to a major axis of the tissue-engaging surface, and (vi) one or more springs, where at least one spring is operably linked to the actuation mechanism and/or the tissue interfacing component.

In some embodiments, the self-righting article comprises (i) one or more curved surfaces adjacent a tissue-engaging surface, (ii) a drug-containing portion, wherein the drug-containing portion occupies greater than or equal to 30 vol % of the self-righting article, wherein the drug-containing portion comprises a plurality of tissue interfacing components, (iii) an actuation mechanism configured to actuate horizontally with respect to a major axis of the tissue-engaging surface, and (iv) one or more springs, where at least one spring is operably linked to the actuation mechanism and/or the plurality of tissue interfacing components.

In some embodiments, the self-righting article comprises (i) one or more curved surfaces adjacent a tissue-engaging surface, (ii) a drug-containing portion, wherein the drug-containing portion comprises greater than or equal to 250 uL of drug, (iii) a needle, (iv) an actuation mechanism configured to actuate horizontally with respect to a major axis of the tissue-engaging surface, and (v) one or more springs, where at least one spring is operably linked to the actuation mechanism and/or the needle.

In some embodiments, the self-righting article comprises (i) one or more curved surfaces adjacent a tissue-engaging surface, (ii) a drug-containing portion, wherein the drug-containing portion comprises greater than or equal to 250 uL of drug, (iii) a liquid jet injection component, (iv) an actuation mechanism configured to actuate horizontally with respect to a major axis of the tissue-engaging surface, and (vi) one or more springs, where at least one spring is operably linked to the actuation mechanism and/or the jet injection component.

In some embodiments, the self-righting article comprises an outer shell, an active pharmaceutical ingredient disposed within the outer shell configured for delivery to a location internal to a subject, the outer shell comprising (i) a flat surface, (ii) a lateral monostable cross-section, and (iii) a longitudinal, bistable, cross-section, such that the outer shell reorients such that the flat surface contacts a surface, regardless of the initial orientation of the self-righting article.

Other advantages and novel features of the present invention will become apparent from the following detailed description of various non-limiting embodiments of the invention when considered in conjunction with the accompanying figures. In cases where the present specification and a document Incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A provides a cross-sectional schematic diagram of an exemplary self-righting article showing a side view of lateral self-orientation and stability, according to one set of embodiments;

FIG. 1B provides a cross-sectional schematic diagram of an exemplary self-righting article showing a side view of longitudinal self-orientation and stability, according to one set of embodiments;

FIG. 1C provides a cross-sectional schematic diagram of an exemplary self-righting article showing self-righting behavior, according to one set of embodiments;

FIG. 2 provides a cross-sectional schematic diagram of an exemplary self-righting article comprising a horizontal liquid injector, according to one set of embodiments;

FIG. 3 provides a cross-sectional schematic diagram of an exemplary self-righting article comprising a microneedle injector, according to one set of embodiments;

FIG. 4 provides a cross-sectional schematic diagram of an exemplary self-righting article comprising a horizontal liquid jetting injector, according to one set of embodiments

FIG. 5 provides a schematic illustrating the self-righting behavior and autonomous triggering and delivery of drug into the submucosal space of the GI tract using an exemplary self-righting article comprising a horizontal liquid injector; according to one set of embodiments;

FIG. 6 provides a schematic illustrating the self-righting behavior and autonomous triggering and delivery of drug into the submucosal space of the GI tract using an exemplary self-righting article comprising a microneedle injector; according to one set of embodiments;

FIG. 7 illustrates a cross-sectional schematic diagram of an exemplary self-righting article comprising a horizontal liquid injector, an oblique projection of the self-righting article using computer aided drawing (CAD), and photographs of the various internal and exterior components of the self-righting article, according to one set of embodiments;

FIG. 8 illustrates a cross-sectional schematic diagram of an exemplary self-righting article comprising a microneedle injector, an oblique projection of the self-righting article using computer aided drawing (CAD), and photographs of the various internal and exterior components of the self-righting article, according to one set of embodiments;

FIG. 9 illustrates the self-righting mechanism of an exemplary horizontal liquid injector, according to one set of embodiments. When a major longitudinal cross-section of the device is substantially perpendicular to an exterior tissue surface (e.g., surface of stomach or GI tract), a longitudinal cross-sectional dimension of the device is configured to orient to the major longitudinal axis of the device parallel to the exterior surface (e.g., surface of the stomach or GI tract). Thereinafter, a lateral cross-sectional dimension of the device is configured to orient the device such that a tissue-engaging surface of the device is in contact with the exterior tissue.

FIGS. 10A and 10B further illustrate the self-righting mechanism shown in FIG. 9, according to one set of embodiments;

FIGS. 11A and 11B illustrate the self-righting mechanism of an exemplary horizontal microneedle injector, according to one set of embodiments;

FIGS. 12A and 12B illustrate the self-righting mechanism of an exemplary horizontal liquid jetting injector, according to one set of embodiments;

FIGS. 13A-13C illustrate photomicrographs of an exemplary horizontal liquid injector (FIG. 13A), an exemplary horizontal microneedle injector (FIG. 13B), and an exemplary horizontal liquid jetting injector within in the stomach of pigs, respectively, according to some embodiments;

FIG. 14 illustrates the in vivo triggering of an exemplary horizontal liquid injector within the stomach of a pig, according to one set of embodiments. After triggering, the exposed needle penetrates the underlying tissue and delivers its liquid cargo into the tissue. The exposed needle and the resultant puncture hole are clearly visible in FIG. 14 (top). FIG. 14 also shows an x-ray of the device within the pigs stomach following deployment;

FIGS. 15A and B illustrates two exemplary embodiments of a horizontal liquid jet injector, according to some embodiments;

FIGS. 16A-G illustrates exemplary gastrointestinal drug delivery devices and gastric tissue structure contemplated herein. FIG. 16A shows a schematic adapted from Abramson et al. illustrating the device pathway in the gastrointestinal tract, including self-orientation, needle insertion, needle retraction, and excretion; FIG. 16B shows a schematic adapted from Abramson et al., Science 363, 611-615 (2019) illustrating a solid SOMA device showing size, drug loading, and injection mechanism; FIG. 16C shows a schematic adapted from Abramson et al., Nat. Biotechnol. 40, 103-109 (2022) illustrating a liquid SOMA device illustrating liquid drug formulation, injection, and retraction mechanisms; FIG. 16D shows a schematic adapted from Arrick et al., Nature 636, 481-487 (2024) illustrating a jetting SOMA device showing triggering sequence and internal mechanism; FIG. 16E shows a schematic adapted from Dhalla et al., Drug Deliv. Transl. Res. 12, 294-305 (2021) illustrating device activation via chemical reaction inflating a sealed balloon, aligning a micro syringe to insert a needle into the small intestine wall; FIG. 16F shows a schematic adapted from Palacios et al., J. Control Release (2025) illustrating capsule self-pressurization enabling aerosol-based biologic delivery; FIG. 16G shows a table adapted from Friis et al., J. Mech. Behav. Biomed. Mater. (2023) illustrating a comparative analysis of human and porcine stomachs, showing distinct tissue layers and thicknesses, according to some embodiments;

FIGS. 17A-B: illustrates exemplary OROS capsule dimensions and theoretical model schematic. FIG. 17A shows a schematic adapted from Caffarel-Salvador et al. Curr. Opin. Pharmacol. 36, 8-13 (2017) illustrating OROS capsule diameter and length compared to standard capsule sizes, with diameter matching size 00+ and length matching size 3; FIG. 17B illustrates a simplified schematic of an exemplary jetting device illustrating the theoretical model framework, showing the model inputs alongside the main contributors to hydraulic losses, according to some embodiments;

FIGS. 18A-H show COMSOL geometries and meshes for simplified and full-device nozzle simulations. FIG. 18A shows a 3D geometry of the conic straight circular nozzle; FIG. 18B shows a user-defined mesh for the conic straight circular nozzle; FIG. 18C shows a 3D geometry of the conic straight slot nozzle; FIG. 18D shows a user-defined mesh for the conic straight slot nozzle; FIG. 18E shows a 2D section of the nozzle geometry indicating the parameters subject to optimization (FIGS. 18A-18E refer to the simplified simulation); FIG. 18F shows the full 3D geometry of the external conic straight circular nozzle with optimizable parameters highlighted; FIG. 18G shows the full 3D device with the straight circular nozzle and no internal 90° flow bend; FIG. 18H shows the full 3D device with an integrated cylindrical outlet and optimizable parameters (all geometries and simulations were developed in COMSOL MULTIPHYSics® (version 6.3, COMSOL AB, Stockholm, Sweden)), according to some embodiments;

FIGS. 19A-E show a pneumatic bench setup for nozzle testing and ex vivo depot formation. FIG. 19A shows the complete bench setup with pneumatic actuator connected to the air cylinder, release lever for device triggering, and interchangeable nozzle; a force sensor is positioned beneath the nozzle; FIG. 19B shows the circular nozzle under testing; FIG. 19C shows the slot nozzle under testing; FIG. 19D shows the placement of gastric tissue beneath the pneumatic bench setup for ex vivo studies of depot formation in the gastric antrum; FIGS. 19E-F shows a plot of the force generated during jetting as a function of time (a peak force of 0.63N is required to generate a depot in a pig's stomach, FIG. 19F); FIG. 19G shows a plot of the total volume delivered by jetting, relative to control devices (standard syringe and needle). Jetting devices delivered on average 215 uL per injection compared to controls which only delivered about 206 uL per injection, constituting a volume delivery efficiency of about 82% (n=5), according to some embodiments;

FIGS. 20A-D show CAD models and physical prototypes of exemplary non self-orienting and self-orienting devices contemplated herein. FIG. 20A shows a non self-orienting device showing both the CAD model and physical implementation, including the two nozzle CAD designs used for validation; FIG. 20B shows a device CAD and physical implementation without the 90° internal bend; FIG. 20C shows a self-orienting device with external conic straight circular nozzle and 150 μL drug payload, shown in CAD and physical form; FIG. 20D shows a nozzle-less self-orienting device with 200 μL drug payload, shown in CAD and physical form, according to some embodiments;

FIG. 21A-G shows exemplary experimental setups and workflows for testing exemplary jetting devices contemplated herein. FIG. 21A shows an exemplary force measurement setup using piezoelectric force transducers (Kistler 9215A load cell); FIG. 21B shows an illustration of a high-speed camera setup for evaluating jet velocity, columnarity, and jetting period; FIG. 21C shows an illustration of a high-speed camera setup for assessing ex-vivo depot formation; FIG. 21D illustrates the workflow for micro-CT imaging of ex vivo tissue with a depot; FIG. 21E shows the setup for ex-vivo self-orientation testing with launching platform; FIG. 21F illustrates an exemplary in vitro testing setup with direct jetting into a vial; and FIG. 21G shows a schematic of the workflow for in vivo experiments, according to some embodiments;

FIGS. 22A-D show the results of an exemplary theoretical jetting model according to some embodiments. FIG. 22A shows pressure-displacement curves for different spring configurations tested, with peak input pressures indicated; FIG. 22B shows jet duration as a function of outlet diameter after fine-tuning of the model, for both three- and four-helical spring configurations; FIG. 22C shows piston displacement over time for varying outlet diameters, with dashed lines representing the total stroke length of 4.65 mm; and FIG. 22D shows the instantaneous output jet power for different outlet diameters, according to some embodiments;

FIGS. 23A-H show the simplified simulation results and experimental validation of exemplary jets tested. FIG. 23A shows the jet power over time for 450 μm and 500 μm circular nozzles, confirming the theoretical prediction that jet power increases with outlet area; FIG. 23B shows the simulated jet power for a 450 μm circular nozzle and an equivalent slot nozzle, with the circular nozzle achieving higher jet power; FIG. 23C shows COMSOL-simulated velocity field for a contraction angle of 40°; FIG. 23D shows COMSOL-simulated velocity field for a contraction angle of 75°; FIG. 23E shows jet power for different contraction angles, showing that smaller contraction angles yield higher jet power; FIG. 23F shows force sensor readout for the three-helical-spring configuration across circular and slot nozzles, validating simulation trends; FIG. 23G shows a boxplot of measured jetting period for the three-helical-spring configuration and different nozzles, showing mean and standard deviation; and FIG. 23H shows a boxplot of measured peak jet force for the three-helical-spring configuration and different nozzles, showing mean and standard deviation, according to some embodiments;

FIG. 24A-H shows full 3D simulation results with experimental validation of exemplary jets tested. FIG. 24A shows the simulated jet force for the four-helical-spring configuration with nozzle outlet diameters of 500 μm, 600 μm, and 800 μm; FIG. 24B shows the measured jet force for the same configuration and outlet diameters as in FIG. 24A; FIG. 24C shows a boxplot of measured jetting period for the four-helical-spring configuration and different nozzles, showing mean and standard deviation; FIG. 24D shows a boxplot of measured peak jet force for the four-helical-spring configuration and different nozzles, showing mean and standard deviation; FIG. 24E shows the simulated jet force for the four-helical-spring configuration to assess the internal flow contribution of a 90° bend, comparing results with and without the bend; FIG. 24F shows the measured jet force for the four-helical-spring configuration without the 90° bend; FIG. 24G shows the simulated jet force for the four-helical-spring configuration without the external nozzle, comparing drug loads of 200 μL and 150 μL; and FIG. 24H shows the measured jet force for the four-helical-spring configuration without the external nozzle and a drug load of 200 μL, according to some embodiments;

FIG. 25A-C shows high-speed imaging of jet formation and device recoil. FIG. 25A shows jet formation with the non-self-orienting device equipped with a 600 μm nozzle and three helical springs (sequential frames show jet columnarity and the time required for the tip of the jet to travel the 2 cm offset to the reference plane, 15 ms); FIG. 25B shows jet formation with the non-self-orienting device equipped with a 600 μm nozzle and four helical springs, with frames illustrating jet columnarity and the shorter travel time for the tip of the jet to reach the 2 cm offset (10 ms); and FIG. 25C shows sequential frames showing device triggering and recoil on ex vivo tissue (the third frame highlights minimal recoil, and the fourth frame highlights depot formation at the target site), according to some embodiments;

FIG. 26A-F shows ex vivo depot formation and volumetric analysis. FIG. 26A shows ex vivo depot formation using the pneumatic bench setup, serving as the gold standard for visualizing depot morphology; FIG. 26B shows partial ex vivo depot obtained with the three-helical-spring configuration, indicating the need for increased input pressure; FIG. 26C shows ex vivo depot obtained with the four-helical-spring configuration, showing a large amount of formulation delivered; FIG. 26D shows sequential high-speed camera frames of ex vivo depot formation for the four-helical-spring configuration: at 2.9 ms the device is triggered, and by 24 ms jetting is completed, resulting in the depot shown in c. e, MicroCT scan of the ex vivo depot formed using a 100% iohexol solution, with the depot area appearing brighter than the surrounding tissue; and FIG. 26F shows results from three trials and a control, showing measured depot volume (μL) from MicroCT scans and calculated delivery efficiency based on a loaded volume of 200 μL, according to some embodiments;

FIGS. 27A-D show the self-orientation analysis and experimental validation. FIG. 27A shows a mathematical self-orientation analysis for the three-helical-spring configuration, the four helical-spring configuration, and the four-helical-spring nozzle-less configuration, with the relevant design variables shown for each (the three-helical-spring configuration, tested in vivo, is taken as the benchmark); FIG. 27B shows sequential frames from the ex vivo self-orientation test illustrating the two-phase self-orientation process; FIG. 27C shows all possible device orientations when dropped into the stomach, with the corresponding self-orientation success rate measured over five trials; FIG. 27D shows in vivo experiments with the three-helical spring self-orienting device, showing successful self-orientation in the stomach, whereas the control device (blue) did not self-orient (the in vivo test was repeated twice, yielding consistent results), according to some embodiments; and

FIG. 28A-B show RNA transfection and encapsulation efficiency using exemplary devices contemplated herein. FIG. 28A shows bioluminescence signal 24 h post-transfection for self-orienting devices, jetting control, and pure formulation control, showing a significant drop between the latter two; and FIG. 28B shows encapsulation efficiency for jetting control and four jetting replicates, with similar values across all replicates.

### DETAILED DESCRIPTION

### Overview

Self-righting articles, such as self-righting capsules for administration to a subject, are generally provided. In some embodiments, the self-righting article may be configured such that the article may orient itself relative to a surface (e.g., a surface of a tissue of a subject). The self-righting articles described herein may comprise a tissue engaging surface configured to engage (e.g., interface with or inject into) with a surface (e.g., a surface of a tissue of a subject). For example, the self-righting article may be placed at any orientation proximate a surface and the self-righting article will (re)-orient itself such that the tissue engaging surface is in contact (e.g., direct contact) with the surface. In some embodiments, the self-righting article may have a particular shape and/or distribution of density (or mass) which, for example, enables the self-righting behavior of the article. In some such embodiments, the capsule containing the self-righting article may be administered to a subject (e.g., for delivery of the self-righting article to a location internal of the subject such as the gastrointestinal tract). In some embodiments, the self-righting may comprise a tissue interfacing component and/or a pharmaceutical agent (e.g., for delivery of the active pharmaceutical agent to a location internal of the subject). In some embodiments, upon contact of the tissue with the tissue engaging surface of the article, the self-righting article may be configured to release one or more tissue interfacing components. In some embodiments, the tissue interfacing component is associated with a self-actuating component. For example, the self-righting article may comprise a self-actuating component configured, upon exposure to a fluid, to release the tissue interfacing component from the self-righting article. In some embodiments, the tissue interfacing component may comprise and/or be associated with the pharmaceutical agent (e.g., for delivery to a location internal to a subject).

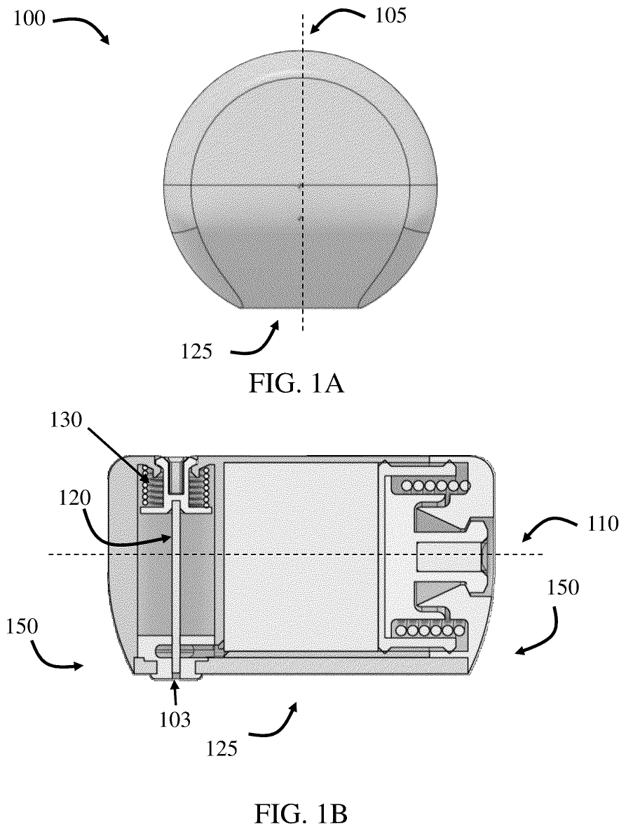

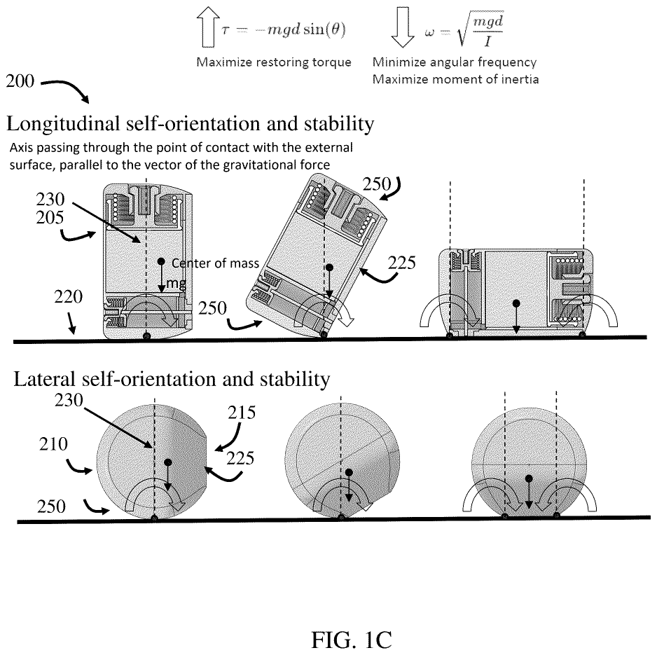

In some embodiments, the self-righting articles contemplated herein utilize a horizontal injector system to deliver a therapeutic payload to a tissue in a subject, for example, stomach tissue and/or a tissue within the gastrointestinal (GI) tract. In some embodiments, the articles disclosed herein advance the field of internal drug delivery devices in several ways. First, in some embodiments, the articles disclosed herein comprise a longitudinal cross-sectional geometry and a lateral cross-sectional geometry, combined with a unique distribution of mass, that directs the device to reliably orient itself in a specific and desired horizontal position, relative to a target tissue. For example, if the device is initially oriented such that a longitudinal axis is not parallel to the target tissue, the longitudinal cross-sections (and distribution of mass across the device) cause the device to “fall over” such that the longitudinal axis is parallel to the tissue. Once in this position, the devices lateral cross-sectional geometry causes the device to rotate onto a flat bottom (e.g., the specific and desired horizontal position) (See e.g., the illustrative embodiments shown in FIGS. 1C and FIGS. 9-12). Advantageously, such a design permits the article to adopt one or more intermediary orientations before self-orienting into a final stable orientation. Second, in some embodiments, the discovery of the above-mentioned advantageous article geometries permits ultra-high drug loading through more efficient utilization of the internal space within the devices. For example, in some embodiments, the devices disclosed herein comprise greater than 30% drug volume relative to device volume, which is about an order of magnitude greater than previously reported injection devices. Third, in some embodiments, the disclosed article geometries advantageously permit miniaturization of the ingestible self-righting devices to a size that is comparable to FDA approved osmotic-controlled release oral delivery systems (OROS). Such size advantageously facilitates patient compliance and improves the safety profile by reducing the risk of obstruction. Fourth, in some embodiments, the disclosed geometries permit the use of various horizontal injector systems and configurations, including but not limited to, liquid injectors, microneedle injectors, and liquid jetting injectors (e.g., needleless injectors), as described in more detail below. Lastly, the articles disclosed herein can be designed to contain a therapeutic agent within a removable drug-loading cartridge configured to be loaded independently from the ingestible device assembly. In this way, the articles disclosed herein can be loaded, and used to deliver, both liquid and solid drug formulations that traditionally could only be delivered via intravenous injection. Advantageously, the articles described herein are capable of delivery of liquid and/or solid therapeutics to various desired and tunable tissue depths.

The skilled artisan will appreciate, based upon the teachings of this specification, that the improved drug loading and miniaturization of the devices disclosed herein makes the disclosed systems capable of delivering clinically relevant doses of macromolecule drugs while maintaining a smaller size that ensures no obstruction through the gastrointestinal tract.

The skilled artisan will also appreciate, based upon the teachings of this specification, that increasing the usage of the internal space within the pill to maximize drug loading is not obvious and requires the development of novel actuation mechanisms which affect the weight balance within the devices and, consequently, require new designs to allow for self-orientation for engagement with the gastrointestinal tract.

Accordingly, some aspects of the disclosure are directed toward the device geometries, for example, that enable horizontal self-righting. Additionally, some aspects are directed toward the various injector systems capable of being encapsulated within said devices.

In some embodiments, the self-righting articles disclosed herein further comprise a reservoir comprising a drug-containing portion. In some embodiments, the drug-containing portion comprises a liquid active pharmaceutical agent. In some embodiments, the drug-containing portion comprises a solid active pharmaceutical agent. In some embodiments, a tissue interfacing component is operably linked to an actuation mechanism.

In some embodiments, the self-righting articles disclosed herein comprise a plurality of tissue interfacing components such as an array of microneedles. In some embodiments, each microneedles comprises the same or different active pharmaceutical agent. In some embodiments, the active pharmaceutical agent may be in solid form. In some embodiments, the plurality of tissue interfacing components is operably linked to an actuation mechanism.

The self-righting articles described herein may be useful, for example, as a general platform for delivery of a wide variety of pharmaceutical agents that otherwise are generally delivered via injection directly into tissue due to degradation in the GI tract. In some embodiments, the self-righting article may be configured to deliver pharmaceutical agents at a desired location and/or at a desired time and/or over a desired duration to a subject. In some embodiments, the self-righting articles described herein may be used to deliver sensors and/or take biopsies, for example, without the need for an endoscopy.

Advantageously, in some embodiments, the self-righting articles and/or self-actuating components described herein may be useful as a general platform for delivery of a wide variety of pharmaceutical agents (e.g., APIs) that are typically delivered via injection directly into tissue due to degradation in the GI tract. For example, the self-righting article may be capable of localizing itself to the tissue wall in a specified direction (e.g., allowing loaded drugs to avoid long passages through the GI tract fluid before diffusing into the blood stream). The article, in some embodiments, may serve as a platform to allow drugs that are currently degraded by the enzymes in the GI tract to be absorbed with higher bioavailability. Additionally, the article may enable mechanical mechanisms such as needle plungers, to actuate directly at and/or into the tissue wall. In this way, in some embodiments, the article may serve as a vehicle to deliver articles into the GI tract.

In some embodiments, the tissue interfacing component (e.g., associated with a self-actuating component) may comprise a relatively high loading of active pharmaceutical ingredients (e.g., drugs). For example, In some embodiments, the tissue interfacing component comprises a solid therapeutic agent (e.g., a solid API) and, optionally, a support material (e.g., a binder such as a polymer) such that the solid therapeutic agent is present in the component in a relatively high amount (e.g., greater than or equal to 80 wt %) versus the total weight of the tissue interfacing component. Such tissue-interfacing components may be useful for delivery of API doses (e.g., to a subject). Advantageously, in some embodiments, the reduction of volume required to deliver the required API dose as compared to a liquid formulation permits the creation of solid needle delivery systems for a wide variety of drugs in a variety of places/tissues (e.g., tongue, GI mucosal tissue, skin) and/or reduces and/or eliminates the application of an external force in order to inject a drug solution through the small opening in the needle. In some embodiments, a physiologically relevant dose may be present in a single tissue interfacing component (e.g., having a relatively high API loading).

In an exemplary embodiment, the self-righting article may comprise a tissue interfacing component and a self-actuating component (e.g., comprising a spring and/or a support material) associated with the tissue interfacing component.

As illustrated in FIGS. 1A and 1B, in some embodiments, exemplary system 100 (e.g., self-righting article) comprises a tissue-engaging surface 125. FIG. 1B further illustrates that, in some embodiments, system 100 comprises one or more curved surfaces 150 adjacent the tissue-engaging surface 125. In some embodiments, self-righting article 100 may be designed and configured such that the tissue-engaging surface 125 contacts a surface (e.g., a surface of a tissue at a location internal to a subject such as a surface of a stomach of the subject). In some embodiments, system 100 will self-right (e.g., will orient without the need or use of external forces applied to the self-righting article) such that the tissue-engaging surface 125 contacts the surface. In some embodiments, the self-righting article is configured such that an axis essentially perpendicular to the tissue-engaging surface preferentially aligns parallel to the direction of gravity.

Without wishing to be bound by theory, the self-righting article may be designed to self-right as a result of the shape of the article and/or distribution of densities (and/or masses) within the self-righting article. For example, in some embodiments, self-righting system 200 (e.g., self-righting article) comprises a tissue-engaging surface 225 and one or more curved surfaces 250 adjacent the tissue-engaging surface 125. In some embodiments, self-righting article 200 comprises a first portion 210 and a second portion 215, the first portion and the second portion having different densities and/or different masses. Different densities/masses of the self-righting article are described in more detail herein. In some embodiments, the self-righting article may have a particular shape which enables the self-righting behavior. For example, as illustrated in FIG. 1C, system 200 comprises a self-righting shape as indicated by external surface 205 of self-righting article 200. In some embodiments, when the self-righting article is oriented in a longitudinal orientation, a center of mass of the self-righting article is offset from a first axis 230 that passes through the center of curvature of the one or more curved surface 250, and is perpendicular to tissue engaging surface 225. Alternatively, in some embodiments, when the self-righting article is oriented in a longitudinal orientation, a center of mass of the self-righting article is offset from a first axis 230 that passes through the point of contact with the external surface 220 and is parallel to the vector of the gravitational force.

In some embodiments, the self-righting device rotates toward the center of mass until the tissue engaging surface contacts external surface 220. Similarly, in some embodiments, when the self-righting article is oriented in a lateral orientation, a center of mass of the self-righting article is offset from a first axis 230 that passes through the center of curvature of the one or more curved surface 250, and is perpendicular to tissue engaging surface 225. Alternatively, in some embodiments, when the self-righting article is oriented in a lateral orientation, a center of mass of the self-righting article is offset from a first axis 230 that passes through the point of contact with the external surface 220 and is parallel to the vector of the gravitational force. In some embodiments, the self-righting device rotates toward the center of mass until the tissue engaging surface contacts external surface 220.

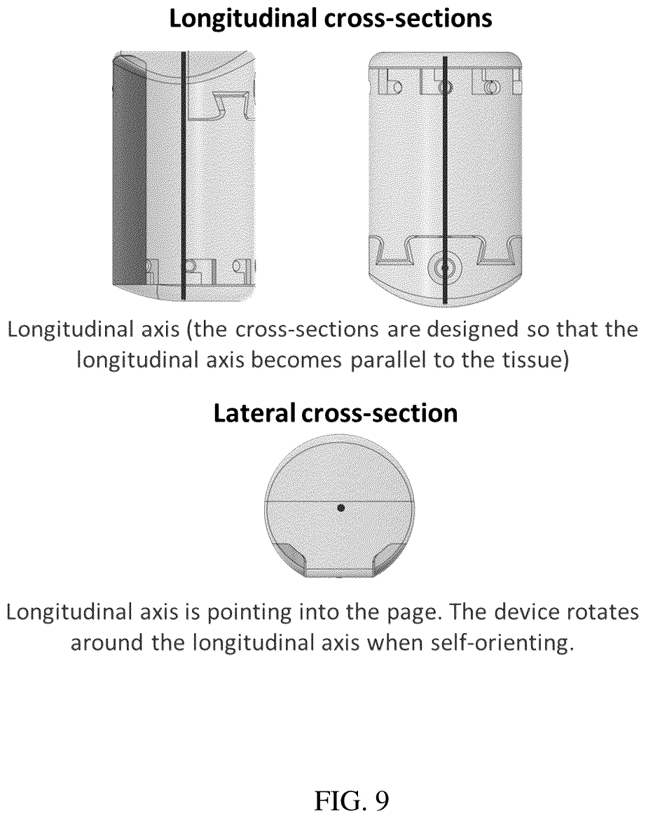

In some embodiments, the article comprises a longitudinal cross-section and a lateral cross-section. In some embodiments, the longitudinal and lateral cross-sections are designed such that, regardless of the initial orientation of the article, the article self-rights such that a longitudinal axis of the article becomes parallel to the tissue. For example, as illustrated schematically in FIG. 9, in some embodiments, the article comprises a longitudinal axis and an external shape such that, upon contact with an external surface (e.g., a surface of tissue at a location internal to a subject, on a surface external to a subject such as a flat surface), the article reorients (under the force of gravity) such that the longitudinal axis becomes parallel to the surface. In some embodiments, the article rotates (e.g., around the longitudinal axis), such that a flat surface of the article contacts the external surface.

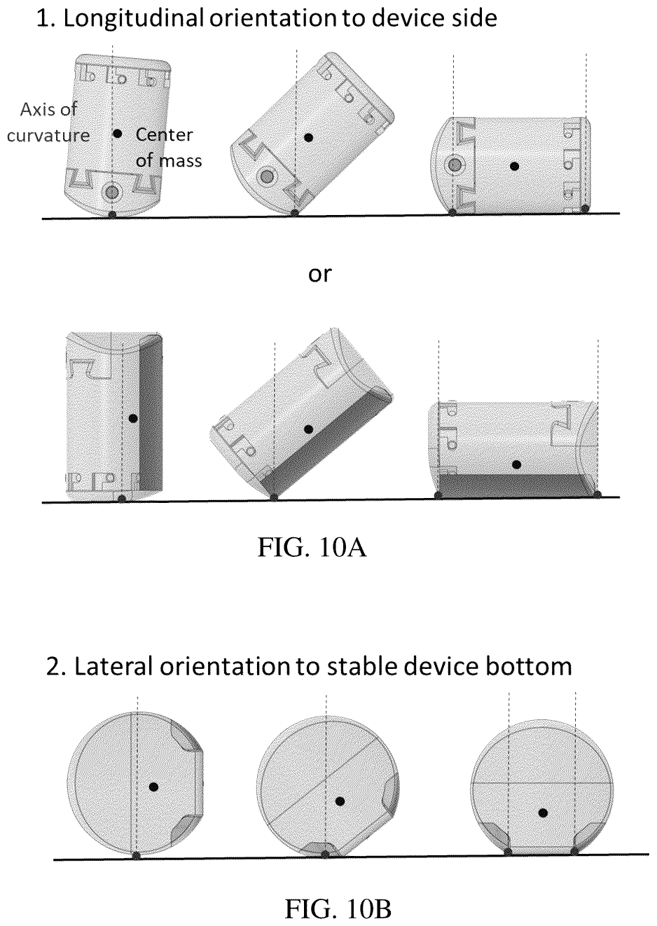

In some embodiments, the self-righting article comprises an outer shell, the outer shell comprising a flat surface, a lateral monostable cross-section, and a longitudinal, bistable, cross-section, such that the outer shell reorients such that the flat surface contacts a surface, regardless of the initial orientation of the self-righting article. For example, in some embodiments as illustrated in FIG. 10A, the outer shell has at least one curved surface (e.g., two curved surfaces) such that the center of mass of the article is offset from an axis of curvature of at least a portion of the outer shell. In some embodiments, the article self-rights to obtain a horizontal (lateral) configuration (e.g., such that a longitudinal axis of the article is parallel with an external surface). In some embodiments, as illustrated in FIG. 10B, the center of mass of the article is offset from a lateral axis of curvature such that the article rotates upon obtaining the lateral orientation. In some such embodiments, the article rotates until on a flat surface of the article contacts an external surface (e.g., a surface of tissue at a location internal to a subject, on a surface external to a subject such as a flat surface).

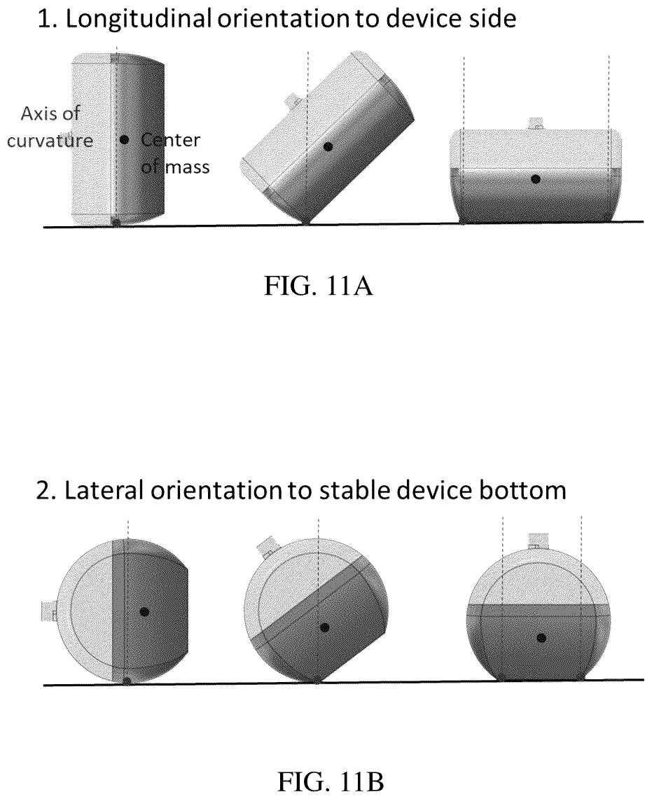

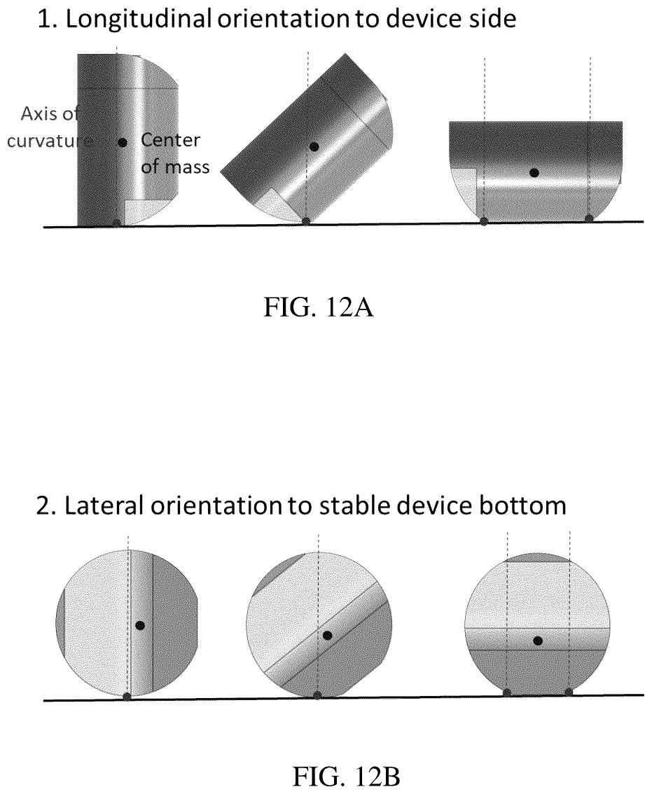

The article may have any suitable outer shell shape and/or cross-sections. FIGS. 11A-12B show additional potential configurations and self-righting behavior of self-righting articles as described herein.

As described above, the devices, systems, and methods disclosed herein comprise one or more self-actuators. The self-actuators may be configured, e.g., upon exposure to a particular fluid, to release a tissue interfacing component associated with a self-actuating component, from system. For example, In some embodiments, a self-actuating component comprises a spring such that, upon actuation of the self-actuating component, the spring expands pushing the tissue interfacing component out of the system through a hole (associated with the tissue engaging surface). In some embodiments, the spring comprises a support material which maintains the spring under compression (e.g., under at least 5% compressive strain). In some embodiments, upon exposure of the support material and/or the spring to a fluid, the spring (e.g., a first spring, second spring, third spring, etc.) may be configured to release at least 10% (e.g., at least 20%, at least 30%, at least 40%, at least 50%, at least 60%, at least 70%, at least 80%, at least 90%, including any percentage therein) of a stored compressive energy of the spring (e.g., such that the tissue interfacing component is released). In some embodiments, the spring is associated with the support material (e.g., at least partially encapsulated by the support material, in direct contact with the support material).

In some embodiments, the systems disclosed herein comprise a tissue interfacing component (e.g., microneedle array 415) comprises an active pharmaceutical agent. In some embodiments, the active pharmaceutical agent may be present in the tissue interfacing component at relatively high amounts (e.g., greater than or equal to 10 wt %, greater than or equal to 80 wt %, or greater than or equal to 90 wt % API versus the total weight of the tissue interfacing component). The self-righting articles described herein may, in some embodiments, be administered to a subject e.g., such that the pharmaceutical agent is delivered to the subject. For example, In some embodiments, the article may be administered to the subject and a pharmaceutical agent is released from the article at a location internal to the subject. Administration of the articles and release of pharmaceutical agents are described in more detail herein.

In some embodiments, the system is administered to a subject (e.g., orally). In some embodiments, the system may be administered orally, rectally, vaginally, nasally, or uretherally. In some embodiments, upon reaching a location internal to the subject (e.g., the gastrointestinal tract), at least a portion of a support material degrades such that a spring extends and/or a tissue interfacing component interfaces (e.g., contacts, penetrates) with a tissue located internal to the subject. In some embodiments, the location internally of the subject is the colon, the duodenum, the ileum, the jejunum, the stomach, or the esophagus. In some embodiments, the location internally of the subject is in the buccal space, in the venous system (e.g., an artery), in the respiratory system (e.g., lung), in the renal system, in the urinary system, or in the gastrointestinal system. As described above and herein, in some embodiments, an active pharmaceutical ingredient may be released during and/or after penetrate of the tissue located internal to the subject.

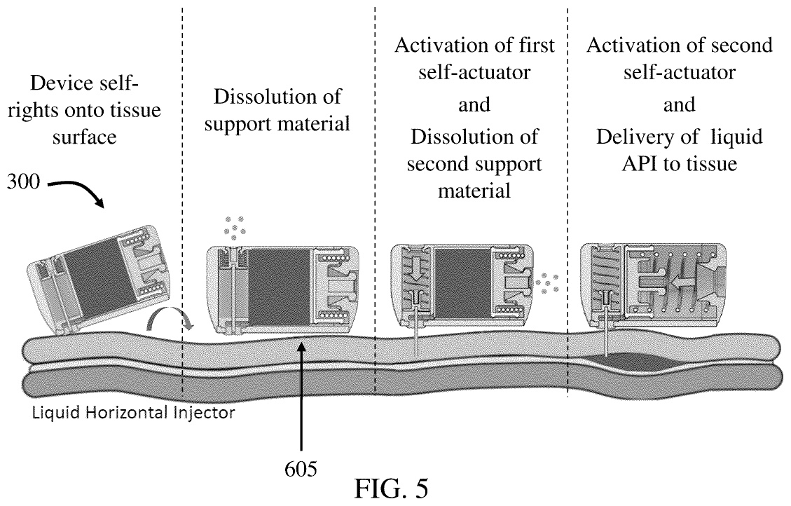

By way of example, and without wishing to be limited by such an exemplary set of embodiments, the system may be administered to a subject orally where it, in some embodiments, travels to the stomach of the subject, sinks to the bottom of the subject's stomach, and the system self-rights such that a tissue-engaging surface of the system contacts the stomach tissue (e.g., the system is at least partly supported by the stomach tissue). For example, as illustrated schematically in FIG. 5, exemplary system 300 may be administered to a subject (e.g., orally) such that system 300 enters gastrointestinal system of the subject. System 300 may travel through gastrointestinal system until reaching stomach of the subject. In some embodiments, system 300 may sink to the bottom of the stomach such that it contacts a surface of stomach 605. In some embodiments, system 300 self-rights such that tissue engaging surface 325 of system 300 contacts the surface of stomach 605 and system 300 self-actuates such that needle 320 (e.g., tissue interfacing component) interfaces with a tissue at a location internal to a subject (e.g., the surface of stomach 605). While FIG. 5 illustrates interfacing of the tissue interfacing component with surface of the stomach 605, those of ordinary skill in the art would understand, based upon the teachings of this specification, that the tissue interfacing component may contact one or more layers underlying the surface of the stomach (or other location internal to the subject) including e.g., mucosal, sub-mucosal, and/or muscular tissue layer(s).

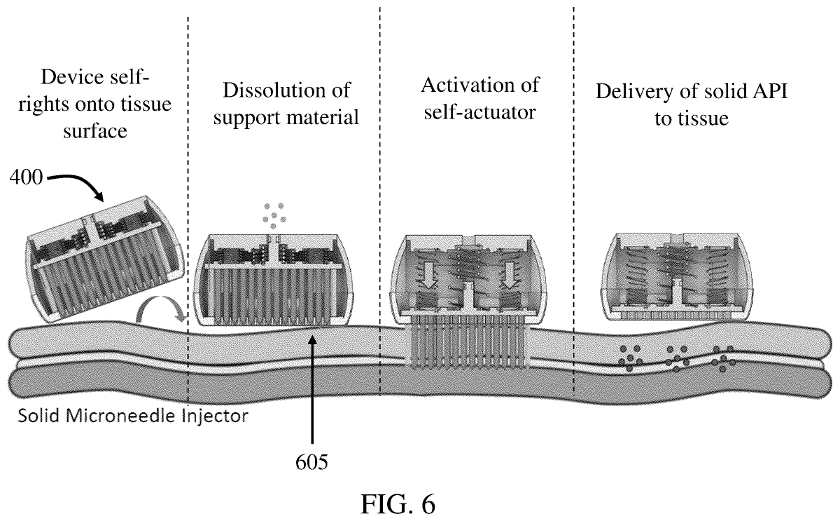

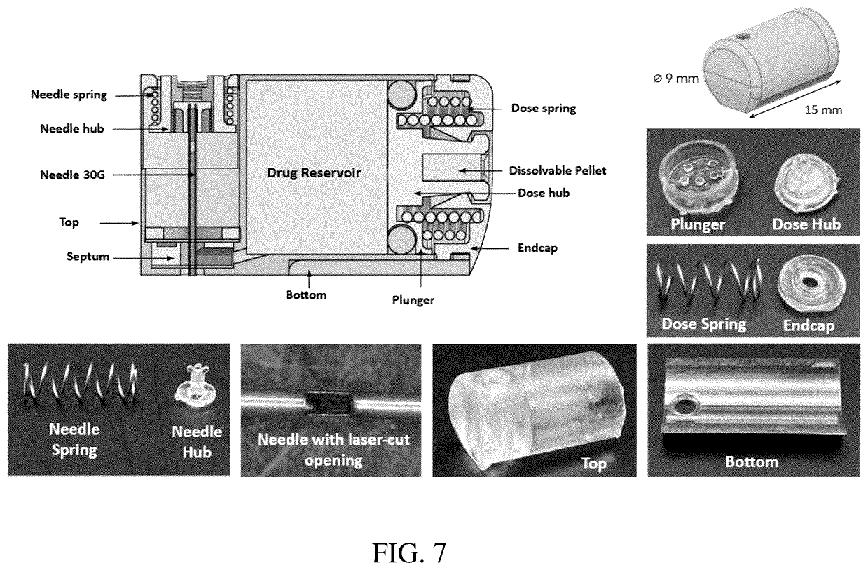

Similarly, FIG. 6, illustrates exemplary system 400 traveling through the gastrointestinal system until reaching the stomach of a subject. In some embodiments, system 400 may sink to the bottom of the stomach such that it contacts a surface of stomach 605. In some embodiments, system 400 self-rights such that microneedle array 415 (e.g., (tissue engaging surface) of system 400 contacts the surface of stomach 605 and system 400 self-actuates such that microneedle array 415 (e.g., tissue interfacing component) interfaces with a tissue at a location internal to a subject (e.g., the surface of stomach 605). While FIG. 6 illustrates interfacing of the tissue interfacing component with surface of the stomach 605, those of ordinary skill in the art would understand, based upon the teachings of this specification, that the tissue interfacing component may contact one or more layers underlying the surface of the stomach (or other location internal to the subject) including e.g., mucosal, sub-mucosal, and/or muscular tissue layer(s).

In some embodiments, as described herein, the self-righting systems disclosed herein are driven by gravitational forces (e.g., acting on a center of mass of system). After a desired period, in some embodiments, the self-righting device disengages (e.g., tissue interfacing component 415 dissolves and/or is released) and exits the body via the stomach. The description above is not meant to be limiting and those of ordinary skill in the art would understand that other interactions between the system and the gastrointestinal system of a subject are also possible, as described herein.

The following description provides various embodiments for the liquid horizontal injection systems, solid microneedle injection systems, and liquid jetting injection systems described herein.

### Liquid Horizontal Injection Systems

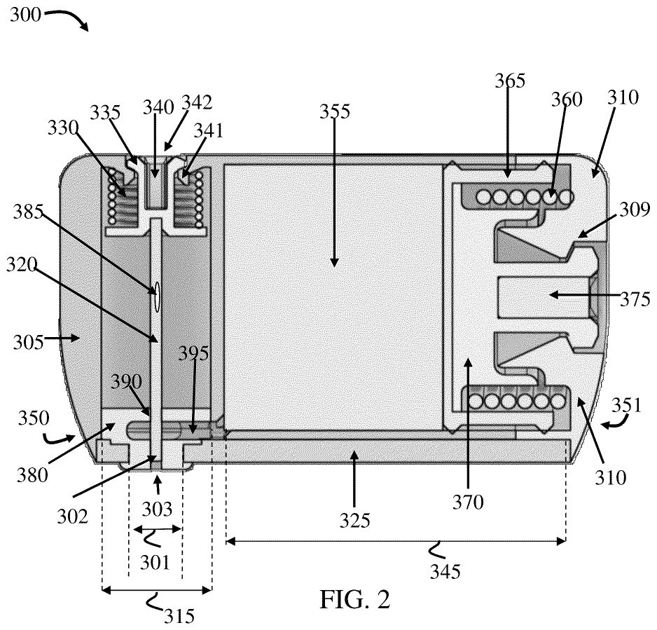

In some embodiments, the articles, systems, and methods disclosed herein are directed to liquid horizontal injection systems. Referring again to FIG. 2, FIG. 2 shows a cross-sectional illustration of exemplary system 300 (e.g., self-righting article) comprising a horizontal liquid injector. In some embodiments, system 300 comprises top 305, endcap 310, and bottom 325 (e.g., tissue-engaging surface). In some embodiments, bottom 325 (e.g., tissue engaging surface) comprises hole 301 configured to receive septum 380. In some embodiments, top 305 comprises hole 342. In some embodiments, top 305 comprises a first curved surface 350 adjacent the bottom 325 (e.g., tissue-engaging surface). In some embodiments endcap 310 comprises a second curved surface 351 adjacent the bottom 325. In some embodiments, system 300 comprises a first reservoir 315 that houses tissue interfacing component 320, a first self-actuator, a first spring 330, needle hub 335, and support material 340 (e.g., isomalt). In some embodiments, needle 320 comprises side hole 385 and a first end 302. In some embodiments, side hole 385 is in fluidic communication with first end 302. In some embodiments, septum 380 further comprises first channel 390 that extends from a first end of septum 380 to a second end 303 of septum 380 and is configured to receive needle 320. In some embodiments, septum 380 comprises a second channel 395 in liquid communication with liquid drug reservoir 355. In some embodiments, needle 320 is operably linked to hub 335, first spring 330, and/or the first self-actuator. In some embodiments, hub 335 is operably linked to needle 320, first spring 330, and/or the first self-actuator. In some embodiments, first spring 330 is operably linked to needle 320, hub 335, and/or the first self-actuator.

In some embodiments, system 300 further comprises second reservoir 345 that houses liquid drug reservoir 355, a second self-actuator, second spring 360, plunger 365, second hub 370, and second support material 375. In some embodiments, liquid drug reservoir 355 is adjacent to and in contact with plunger 365. In some embodiments, plunger 365 is operably linked to second hub 370, second spring 360 and/or the second self-actuator. In some embodiments, second hub 370 is operably linked to plunger 365, second spring 360 and/or the second self-actuator. In some embodiments, second spring 360 is operably linked to second hub 370, plunger 365 and/or the second self-actuator.

In some embodiments, the first self-actuator comprises latch 341 configured to receive a first end of hub 335 (opposite the end configured to receive needle 320). The first end of hub 335 is further configured to receive support material 340 (e.g., the trigger), such that when support material 340 is loaded within the first end of hub 335, the first end of hub 335 engages latch 341, thus anchoring first spring 330 in a first position. In some embodiments, the first position is when first spring 330 is under tension and needle 320 is inside first reservoir 315. Upon dissolution of support material 340 (e.g., isomalt), the first end of hub 335 disengages from latch 341 and the tension applied to first spring 330 drives the needle into a second position. In some embodiments, when in the second position side hole 385 of needle 320 is in fluidic communication with liquid reservoir 355 and first end 302 of needle 320 exits the device through second end 303 of septum 380 into a space exterior of the device.

In some embodiments, the second self-actuator comprises latch 309 (which is comprised within endcap 310 in FIG. 2) configured to receive first end of second hub 370 (opposite the end configured to contact plunger 365). In some embodiments, plunger 365 and/or second spring 360 is operably linked to second hub 370. In some embodiments, the first end of second hub 370 is further configured to receive support material 375 (e.g., a trigger such as isomalt), such that when support material 375 is loaded within the first end of second hub 370, the first end of second hub 370 engages latch 309, thus anchoring plunger 365 and/or second spring 360 in a first position. In some embodiments, when in the first position, second spring 360 is under tension and plunger 365 is proximate endcap 310. Upon dissolution of support material 375 (e.g., isomalt), the first end of second hub 370 disengages from latch 309 and the tension applied to second spring 360 causes the plunger 365 to move in a horizontal direction that compresses liquid drug reservoir 355.

While the figures herein generally illustrate one or two reservoirs (e.g., liquid drug reservoirs), those of ordinary skill in the art would readily envision based upon the teachings of this specification, that the disclosure is not so limited and that an article described herein may comprise two or more (e.g., three or more, four or more, five or more, six or more, seven or more, etc.) reservoirs. In some embodiments, the article comprises a plurality of reservoirs. In some embodiments, at least a portion of the plurality of reservoirs (e.g., two or more, three or more, etc.) may be in fluidic communication at some time period during the administration of the article. Some embodiments may comprise one or more valves, pumps, and/or conduits to bring two or more reservoirs into fluidic communication.

### Solid Microneedle Injection Systems

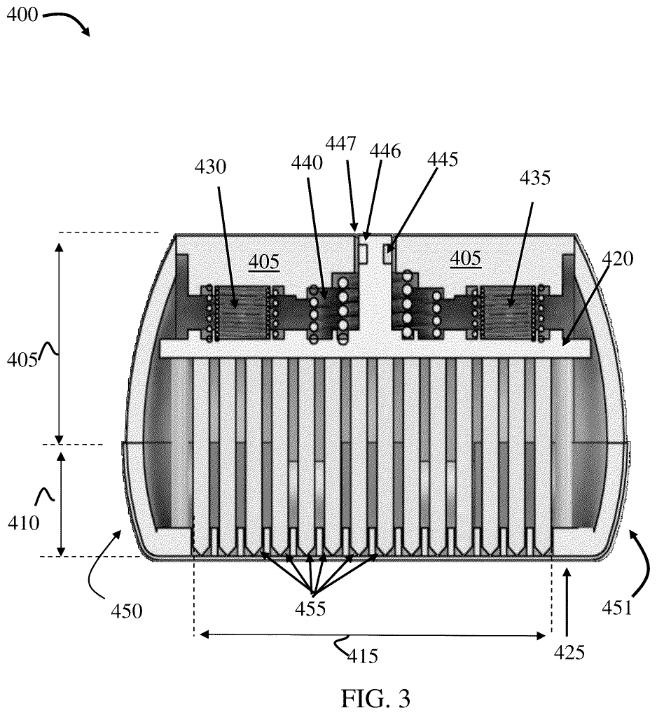

In some embodiments, the articles, systems, and methods disclosed herein are directed to solid microneedle injection systems. FIG. 3 shows a cross-sectional illustration of exemplary system 400 (e.g., self-righting article) comprising a microneedle injector. In some embodiments, the microneedle injector comprises a plurality (e.g., an array) of microneedles. In some embodiments, system 400 comprises top housing 405 and bottom housing 410. In some embodiments, bottom housing 410 comprising a tissue-engaging surface 425. In some embodiments, system 400 comprises first curved surface 450 adjacent tissue engaging surface 425. In some embodiments, system 400 comprises second curved surface 451 adjacent tissue engaging surface 425. In some embodiments, system 400 comprises a single internal reservoir that houses microneedle array 415, a first self-actuator hub 420, first spring 430, second spring 435, third spring 440, and support material 445. In some embodiments, microneedle array 415 comprises a plurality of dissolvable microneedles. In some embodiments, the plurality of dissolvable microneedles comprises an API (e.g., solid API or liquid API). In some embodiments, microneedle array 415 is operably linked to hub 420, first spring 430, second spring 435, third spring 440 and/or the first self-actuator. In some embodiments, first spring 430, second spring 435, and/or third spring 440 are operably linked to hub 420, top 405, and/or the first self-actuator. In some embodiments, hub 420 is operably linked to microneedle array 415, first spring 430, second spring 435, third spring 440, and/or the first-self actuator.

In some embodiments, the first self-actuator comprises a latch. In some embodiments, the latch comprises a plurality of notches 446 located at a first end of hub 420. In some embodiments, the plurality of notches 446 are configured to engage support material 445, wherein the support material 445 is in contact with top 405. In some embodiments, when the plurality of notches 446 engage support material 445, the latch is engaged, thus anchoring first spring 430, second spring 435, third spring 440 in a first position. In some embodiments, the first position is when first spring 430, second spring 435, third spring 440 are loaded (e.g., under compression) and microneedle array 415 is inside first reservoir 415. Upon dissolution of support material 445 (e.g., isomalt), the latch becomes disengaged and the load (e.g., compression) applied to first spring 430, second spring 435, and/or third spring 440 drives the microneedle array 415 into a second position. In some embodiments, bottom housing 410 further comprises a plurality of holes 455, the plurality of holes 455 comprising a plug (e.g., a septum) such that, upon activation of the actuator (e.g., disengagement of the latch), the microneedle array 415 pierces the plug in hole 455 into a space exterior of the device.

### Liquid Jetting Injection Systems

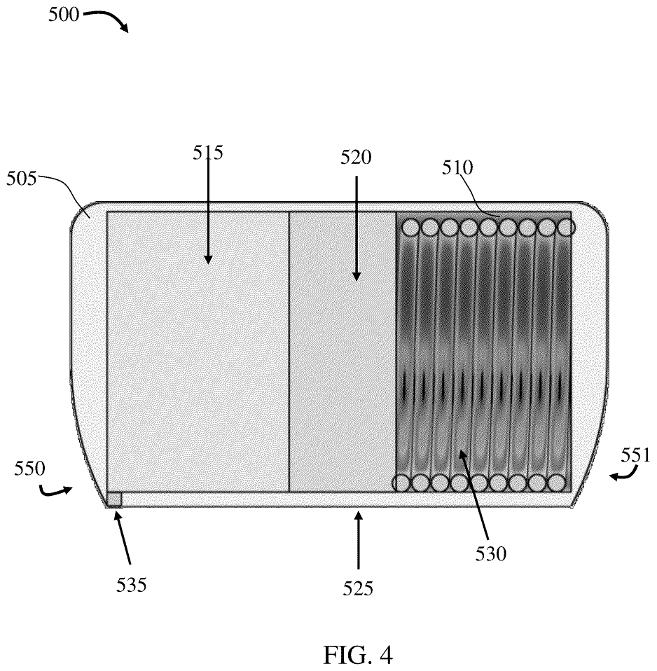

In some embodiments, the articles, systems, and methods disclosed herein are directed to liquid jetting injection systems. FIG. 4 shows a cross-sectional illustration of exemplary system 500 (e.g., self-righting article) comprising a horizontal liquid jet injector. As shown in FIG. 4, system 500 includes a housing 505 containing a potential energy source, configured as a compressed gas compartment 510, and an active pharmaceutical ingredient (API) reservoir 515. The compressed gas compartment 510 further comprises spring 530 operably connected to piston 520, which separates gas compartment 510 from reservoir 515. The piston 520 includes a piston seal (not shown) configured to inhibit fluid transfer between the compressed gas compartment 510 and the reservoir 515. The piston 520 transfers pressure from the compressed gas compartment 510 to the reservoir 515. That is, the compressed gas inside of the gas compartment 510 pressurizes the API disposed inside of the reservoir 515. As shown in FIG. 4, the reservoir 515 includes an outlet 535 in fluid communication with an exterior environment of the device. In some embodiments, the outlet 535 may function as a nozzle for formation of the jet (not shown). The outlet's maximum transverse dimension (e.g. a diameter) may be selected to provide a desired maximum transverse dimension of a corresponding jet that is emitted from the outlet 535 when the pressurized API flows out of the reservoir 515.

The device may also include a trigger that is operatively associated with the potential energy source, which in this case is the pressurized gas compartment 510. In some embodiments, the trigger comprises a support material, as disclosed elsewhere herein (e.g., isomalt). In some embodiments, the trigger is configured to actuate device 500 at a predetermined location within the gastrointestinal tract of a subject such that the potential energy source, which in the current embodiment is the compressed gas compartment 510, compresses the reservoir 515 to deploy a jet of the active pharmaceutical ingredient out of the outlet 535 and into a tissue of a corresponding portion of the gastrointestinal tract located proximate to the device and that the outlet 535 is oriented towards. For example, in the depicted embodiment, the trigger 112 may correspond to a dissolvable plug physically retained within the outlet 535 of the device such that when the trigger is dissolved the device actuates as elaborated on below, though any appropriate trigger may be used as the disclosure is not so limited.

In some embodiments, when a trigger is dissolved, or otherwise actuated inside of a GI tract of the subject, the barrier preventing deployment of the API through the outlet 535 is removed. Accordingly, the pressure applied to the API reservoir 515 by the piston 520 associated with the compressed gas compartment 510 causes the piston 520 to move in a direction that compresses the reservoir 515. As the reservoir 515 is compressed, the API flows out of the outlet 535 in the form of a jet with sufficient velocity to penetrate a tissue of the gastrointestinal tract that is located proximate to the outlet 535. Again, the tissue of the gastrointestinal tract may correspond to the stomach, small intestine, and/or any other anatomical structure of the gastrointestinal tract of a subject described herein. Depending on the embodiment, the jet may form a depot of the API within the tissue of the gastrointestinal tract without perforating the gastrointestinal tract. For example, the outlet 535, API reservoir 515, and associated potential energy source (e.g. the compressed gas compartment 510) may be appropriately configured to provide a jet that is optimized to form a depot corresponding to a volume of the API disposed within the submucosal tissue of the gastrointestinal tract without perforating a muscularis layer of the gastrointestinal tract. Further, depending on the specific operating parameters, the depot may be at least partially disposed in a submucosal tissue and/or the muscularis layer of the gastrointestinal tract.

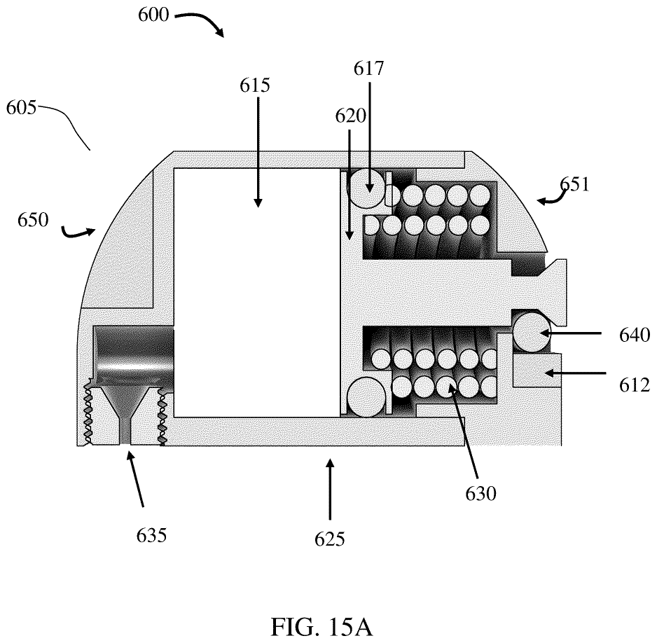

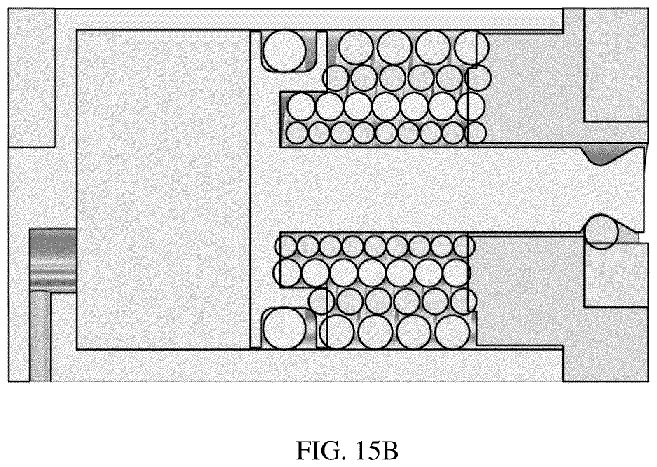

FIG. 15 illustrates another exemplary embodiment of horizontal liquid jet injector 600. In some embodiments, system 600 includes a housing 605 containing a potential energy source, configured as a compressed piston assembly 620, and an active pharmaceutical ingredient (API) reservoir 615. The piston assembly 620 further comprises parallel spring 630 operably connected to piston 620, which separates the potential energy source from reservoir 615. The piston 620 includes a piston seal 617 configured to inhibit fluid transfer between the potential energy source and the reservoir 615. The piston assembly 620 transfers pressure from the compressed parallel spring 630 to the reservoir 615. That is, the compressed parallel spring 630 pressurizes the API disposed inside of the reservoir 615. As shown in FIG. 15, the reservoir 615 includes an outlet 635 in fluid communication with an exterior environment of the device. In some embodiments, the outlet 635 may function as a nozzle for formation of the jet. The outlet's maximum transverse dimension (e.g. a diameter) may be selected to provide a desired maximum transverse dimension of a corresponding jet that is emitted from the outlet 635 when the pressurized API flows out of the reservoir 615.

The device may also include a trigger that is operatively associated with the potential energy source, which in this case is the compressed piston assembly 620 comprising compressed parallel springs 630. In some embodiments, the trigger comprises a support material, as disclosed elsewhere herein (e.g., isomalt). In some embodiments, the trigger is configured to actuate device 600 at a predetermined location within the gastrointestinal tract of a subject such that the potential energy source, which in the current embodiment is the compressed piston assembly 620, compresses the reservoir 615 to deploy a jet of the active pharmaceutical ingredient out of the outlet 635 and into a tissue of a corresponding portion of the gastrointestinal tract located proximate to the device and that the outlet 635 is oriented towards. For example, in the depicted embodiment, the trigger 612 may correspond to a dissolvable plug operably linked to latch 640, such that when the trigger is dissolved, latch 640 disengages the piston assembly 620 and the device actuates as elaborated on below, though any appropriate trigger may be used as the disclosure is not so limited.

In some embodiments, when a trigger is dissolved, or otherwise actuated inside of a GI tract of the subject, the barrier preventing deployment of the API through the outlet 635 is removed. Accordingly, the pressure applied to the API reservoir 615 by the piston assembly 620 associated with the compressed parallel spring 630 causes the piston assembly 620 to move in a direction that compresses the reservoir 615. As the reservoir 615 is compressed, the API flows out of the outlet 635 in the form of a jet with sufficient velocity to penetrate a tissue of the gastrointestinal tract that is located proximate to the outlet 635. Again, the tissue of the gastrointestinal tract may correspond to the stomach, small intestine, and/or any other anatomical structure of the gastrointestinal tract of a subject described herein. Depending on the embodiment, the jet may form a depot of the API within the tissue of the gastrointestinal tract without perforating the gastrointestinal tract. For example, the outlet 635, API reservoir 615, and associated potential energy source (e.g. the compressed piston assembly 620 with corresponding compressed parallel spring 630) may be appropriately configured to provide a jet that is optimized to form a depot corresponding to a volume of the API disposed within the submucosal tissue of the gastrointestinal tract without perforating a muscularis layer of the gastrointestinal tract. Further, depending on the specific operating parameters, the depot may be at least partially disposed in a submucosal tissue and/or the muscularis layer of the gastrointestinal tract.

The following description provides various embodiments for the self-righting, self-actuating, and relatively high API loaded components of the various self-orientating horizontal injector systems described herein (e.g., liquid horizontal injector systems, solid microneedle injector systems, and liquid jetting injector systems).

### Self-Orientating Mechanisms

In some embodiments, the articles, systems, and methods disclosed may be configured to self-orient or self-right when displaced from a predetermined orientation. As used herein, “self-orient” and “self-right” refer to the capability of the device to autonomously return toward a stable equilibrium orientation after being tilted, inverted, rotated, submerged, dropped, or otherwise spatially displaced, without requiring manual external repositioning. The predetermined orientation may be defined relative to gravity, buoyancy, magnetic field, aerodynamic flow, an external reference frame, or any combination thereof.

In some embodiments, self-righting may be achieved through passive structural configuration. In some embodiments, the device may include a mass distribution arranged such that the center of gravity is offset relative to a geometric center, buoyant center, or support contact region. By positioning the center of gravity below a pivot point or below a center of buoyancy, a restoring torque may be generated when the device is displaced, thereby causing rotation toward a lower potential energy state. The mass distribution may be fixed, partially movable, fluid-based, elastically biased, or otherwise dynamically responsive to orientation changes.

In some embodiments, buoyancy-driven restoring forces may be employed. The device may include one or more buoyant chambers, selectively weighted regions, or density gradients configured such that inversion causes a shift in buoyant forces relative to gravitational forces. The resulting torque may rotate the device into an upright configuration. Buoyant elements may include sealed air cavities, compressible materials, foams, gas-filled bladders, or selectively floodable compartments.

In some embodiments, geometric shaping of the exterior surface may promote self-righting behavior. For example, curved lower surfaces, rounded profiles, asymmetric contours, or cambered structures may create rolling tendencies that bias the device toward a preferred orientation. Contact surface geometry may be configured to create an unstable equilibrium in non-preferred orientations, thereby encouraging rotation toward a stable equilibrium configuration. The device may include protrusions, skids, fins, ribs, or weighted lobes that shift the effective pivot point during displacement.

Active self-righting mechanisms may also be implemented. In some embodiments, the device may include one or more actuators configured to reposition internal components, shift mass, extend appendages, deploy stabilizers, or otherwise alter the device's moment distribution in response to detected orientation changes. Sensors such as accelerometers, gyroscopes, inclinometers, magnetometers, pressure sensors, or fluid-level sensors may be used to detect deviation from a predetermined orientation. Control circuitry may activate motors, linear actuators, reaction wheels, control moment gyroscopes, shape-memory materials, solenoids, pneumatic elements, hydraulic elements, or other mechanisms to generate corrective torque.

In some embodiments, inertial or gyroscopic stabilization may be employed. A rotating mass may create angular momentum that resists undesired rotation or assists in reorientation when controlled braking or acceleration is applied. Reaction wheels or flywheels may be selectively accelerated or decelerated to induce counter-rotation of the device body. Similarly, internal pendulum systems or suspended masses may shift in response to gravity to create restoring torque.

Magnetic or electromagnetic mechanisms may also be used. Permanent magnets or electromagnets may interact with an external magnetic field to generate orientation bias. In some embodiments, the device may include magnetically responsive materials positioned to align with Earth's magnetic field or with an artificial field source.

Elastic or stored-energy elements may provide restoring force in certain configurations. Springs, torsion members, compliant hinges, elastomeric components, or bistable structures may be configured such that displacement from the predetermined orientation stores potential energy, which is subsequently released to return the device toward equilibrium.

In some embodiments, fluid redistribution mechanisms may be used. Liquid ballast may shift within internal chambers as orientation changes, thereby altering the center of gravity. The ballast may move passively under gravity, through one-way valves, via capillary channels, or through actively pumped systems. Viscous damping may be incorporated to control the rate of return.

It should be understood that the foregoing mechanisms are not mutually exclusive. In some embodiments, two or more restoring mechanisms may operate in combination. For example, a device may employ passive gravitational mass distribution in conjunction with active actuator-based correction, or buoyant stabilization in combination with geometric rolling bias. Hybrid systems may improve reliability, response time, or robustness under varying environmental conditions.

The restoring force or restoring torque generated in the disclosed embodiments may act about one or more rotational axes and may be configured to correct displacement in one, two, or three spatial dimensions. The predetermined orientation may correspond to a single stable equilibrium state or may include multiple stable orientations depending on application requirements.

Unless expressly limited, the self-orienting functionality described herein does not require continuous power input, specific materials, or particular geometric configurations. Any structure, configuration, material property, mass arrangement, control logic, or environmental interaction capable of producing a restoring moment that biases the device toward a desired orientation is considered within the scope of the present disclosure.

As described above, in some embodiments, the self-righting article may comprise two or more portions having different average densities such that, for example, the self-righting article may orient itself substantially perpendicular to the surface (e.g., a surface substantially orthogonal to the force of gravity, a surface of a tissue such as the wall of the gastrointestinal tract). In some embodiments, the self-righting article may have a particular shape which, for example, enables the self-righting behavior of the article. In some embodiments, the self-righting article may be disposed (e.g., encapsulated) in a capsule. In some embodiments, the self-righting article is not provided in a capsule. In some embodiments, the capsule containing the self-righting article may be administered to a subject (e.g., for delivery of the self-righting article to a location internal of the subject such as the gastrointestinal tract). In some embodiments, the self-righting article and/or the capsule may comprise a pharmaceutical agent (e.g., for delivery of the active pharmaceutical agent to a location internal of the subject).

The self-righting articles described herein may be useful, for example, as a general platform for delivery of a wide variety of pharmaceutical ingredients that otherwise are generally delivered via injection directly into tissue due to degradation in the GI tract. In some embodiments, the self-righting articles described herein may be used to deliver sensors and/or take biopsies, for example, without the need for an endoscopy.

Advantageously, the self-righting article may be capable of localizing itself to the tissue wall in a specified direction (e.g., allowing loaded drugs to avoid long passages through the GI tract fluid before diffusing into the blood stream). As described herein, this article, in some embodiments, may serve as a platform to allow drugs that are currently degraded by the enzymes in the GI tract to be absorbed with higher bioavailability. Additionally, the article may enable mechanical and electrical mechanisms such as needle plungers, anchors, sensors, etc., to actuate directly at and/or into the tissue wall. In this way, in some embodiments, the article may serve as a vehicle to deliver electronics or other articles into the GI tract.

In some embodiments, the self-righting article may have a particular cross-sectional shape. In some embodiments, the shape may be any suitable cross-sectional shape including circular, oval, triangular, irregular, trapezoidal, square or rectangular, or the like.

In some embodiments, the self-righting article is non-spherical. For example, as illustrated in FIG. 1A-1C, the self-righting article comprises one or more curved surfaces adjacent a tissue-engaging surface, wherein the tissue-engaging surface 225 is flat. In some embodiments, the non-spherical shape enables the self-righting behavior. For example, as illustrated in FIG. 1C, system 200 comprises a self-righting shape as indicated by external surface 205 of self-righting article 200. In some embodiments, when the self-righting article is oriented in a longitudinal orientation, a center of mass of the self-righting article is offset from a first axis 230 that passes through the point of contact with the external surface 220 and is parallel to the vector of the gravitational force. In some embodiments, the self-righting device rotates toward the center of mass until the tissue engaging surface contacts external surface 220.

Similarly, in some embodiments, when the self-righting article is oriented in a lateral orientation, a center of mass of the self-righting article is offset from a first axis 230 that passes through the point of contact with the external surface 220 and is parallel to the vector of the gravitational force. In some embodiments, the self-righting device rotates toward the center of mass until the tissue engaging surface contacts external surface 220.

Referring again to FIG. 1C, in some embodiments, self-righting article comprises a first portion 210 and a second portion 215 adjacent first portion 210, having a different average density than the first portion and/or a different mass than the first portion. For example, in some embodiments, the self-righting article comprises a first portion and a second portion adjacent the first portion having a different average density in the first portion. For example, the first portion may have a first average density and a second portion may have a second average density, different than the first average density. Without wishing to be bound by theory, the self-righting article having a first portion and a second portion having different average densities may result in the self-righting article substantially maintaining a particular orientation(s) relative to the surface (e.g. a wall of the gastrointestinal track).

As used herein, when a portion is referred to as being “adjacent” another portion, it can be directly adjacent to (e.g., in contact with) the portion, or one or more intervening components (e.g., a liquid, a reservoir) also may be present. A portion that is “directly adjacent” another portion means that no intervening component(s) is present.

In some embodiments, the self-righting article is configured to be administered directly to a subject (e.g., without encapsulation in a capsule). In some embodiments, the self-righting article is configured and arranged to be encapsulated in a capsule having a shell. In some embodiments, a tissue interfacing component and/or an active pharmaceutical ingredient may be disposed within a reservoir.

In some embodiments, the capsule is a 000 capsule or smaller (e.g., the capsule has a shape or size as described in the USP including, but not limited to, 000 capsule, 00 capsule, 0 capsule, 1 capsule, 2 capsule, 3 capsule, 4 capsule, or 5 capsule.) In some embodiments, the capsule at least partially encapsulates the first portion and the second portion of the self-righting article. In some embodiments, multiple devices can be placed inside of a capsule.

In some embodiments, although the self-righting article may be configured for potential encapsulation in a 000 capsule, or smaller, the self-righting article does not necessarily need to be encapsulated in such capsule. In embodiments wherein the self-righting article is to be administered, such as by ingesting the self-righting article, the self-righting article may thus be administered without encapsulation.

In some embodiments, the capsule is similar in size in at least one dimension to FDA approved osmotic-controlled release oral delivery systems (OROS). OROS are art recognized delivery systems having an overall dimension of about 9 mm×15 mm. In some embodiments, the average cross-sectional dimension of the OROS is greater than or equal to 5 mm, greater than or equal to 6 mm, greater than or equal to 7 mm, greater than or equal to 8 mm, greater than or equal to 9 mm, greater than or equal to 10 mm, greater than or equal to 11 mm, greater than or equal to 12 mm, greater than or equal to 13 mm, greater than or equal to 14 mm, or greater than or equal to 15 mm. In some embodiments, the average cross-sectional dimension is less than or equal to 15 mm, less than or equal to 14 mm, less than or equal to 13 mm, less than or equal to 12 mm, less than or equal to 11 mm, less than or equal to 10 mm, less than or equal to 9 mm, less than or equal to 8 mm, less than or equal to 7 mm, less than or equal to 6 mm, or less than or equal to 5 mm. Combinations are also possible in some embodiments. For example in some embodiments, the average cross-sectional dimension is greater than or equal to 5 mm and less than or equal to 15 mm.

In some embodiments, the average overall length of OROS is between 10 mm and 20 mm. In some embodiments, the overall length is greater than or equal to 10 mm, greater than or equal to 11 mm, greater than or equal to 12 mm, greater than or equal to 13 mm, greater than or equal to 14 mm, greater than or equal to 15 mm, greater than or equal to 16 mm, greater than or equal to 17 mm, greater than or equal to 18 mm, greater than or equal to 19 mm, or greater than or equal to 20 mm. In some embodiments, the average cross-sectional dimension is less than or equal to 20 mm, less than or equal to 19 mm, less than or equal to 18 mm, less than or equal to 17 mm, less than or equal to 16 mm, less than or equal to 15 mm, less than or equal to 14 mm, less than or equal to 13 mm, less than or equal to 12 mm, less than or equal to 11 mm, or less than or equal to 10 mm. Combinations are also possible in some embodiments. For example, in some embodiments, the average cross-sectional dimension is greater than or equal to 10 mm and less than or equal to 20 mm.

In some embodiments, a self-righting device comprises a loading efficiency of between 10% and 50%. A “loading efficiency” as used herein refers to the ratio of the volume of drug occupied within the device relative to the device's total internal volume (e.g., volume of drug/volume of device). In some embodiments, the loading efficiency is greater than or equal to 10%, greater than equal to 20%, greater than or equal to 30%, greater than or equal to 40%, or greater than or equal to 50%. In some embodiments, the loading efficiency is less than or equal to 50%, less than or equal to 40%, less than or equal to 30%, less than or equal to 20%, or less than or equal to 10%. Combinations are also possible in some embodiments. For example, in some embodiments, the loading efficiency is greater than or equal to 10% and less than or equal to 50%. Other ranges are also possible.

In some embodiments, the self-righting article may comprise a coating on at least a portion of an outer surface of the self-righting article. In some embodiments, the system (e.g., the system comprising the self-righting article) comprises a coating (e.g., a film disposed on at least a surface of the system). In some embodiments, the coating may be applied as an aqueous or organic solvent-based polymer system, fats and/or wax. In some embodiments, the coating comprises one or more of a polymer, a plasticizer, a colorant, a solvent, a fat, and a wax. Non-limiting examples of suitable fats and/or waxes include beeswax, carnauba wax, cetyl alcohol, and cetostearyl alcohol.

Non-limiting examples of suitable polymers for the coating include of cellulosic (e.g. hydroxypropylmethylcellulose, hydroxypropylcellulose, hydroxyethylcellulose, hydroxyethylcellulose phthalate, ethylcellulose, cellulose acetate phthalate, cellulose acetate trimellitate), vinyl (e.g. poly(vinyl pyrrolidone), poly(vinyl alcohol), poly(vinyl pyrrolidone)-poly(vinyl acetate)copolymers, poly(vinyl alcohol)-poly(ethylene glycol) co-polymers, poly(vinyl acetate phthalate), glycols (e.g. poly(ethylene glycol)), acrylics (e.g. amino alkyl methacrylate copolymers), other carbohydrates (e.g. maltodextrin, polydextrose), and combinations thereof.

Non-limiting examples of suitable colorants include natural pigments (e.g. riboflavin, beta-carotene, carmine lake), inorganic pigments (e.g. titanium dioxide, iron oxides), water-soluble dyes (FD&C Yellow #5, FD&C blue #2), FD&C lakes (FD&C Yellow #5 Lake, FD&C Blue #2 Lake), and D&C lakes (D&C Yellow #10 Lake, D&C Red #30 Lake).

Non-limiting examples of suitable plasticizers include polyhydric alcohols (e.g. propylene glycol, glycerol, polyethylene glycols), acetate esters (e.g. triacetin, triethyl citrate, acetyl triethyl citrate), phthalate esters (e.g. diethyl phthalate), glycerides (e.g. acylated monoglycerides) and oils (e.g. castor oils, mineral oils).

Polymers, plasticizers, colorants, solvents, fats, and/or waxes may be combined in any suitable amount to form the coating. The coating may be applied in any suitable method including, for example, dip coating and/or spray atomization. Other methods of depositing the coating are also possible.

In some embodiments, a tissue interfacing component is associated with the self-righting article. Non-limiting examples of tissue interfacing components include needles (e.g., stainless steel needles, needles comprising an API), biopsy punches, microneedles (e.g., microneedles comprising an API), projectiles, or the like.

In some embodiments, the tissue interfacing component comprises a jet injection component (e.g., for liquid jet injection using high velocity stream into a tissue of a subject). In an exemplary embodiment, the jet injection component comprises a reservoir comprising a polymeric portion. In some embodiments, the polymeric portion may comprise an acid (e.g., a weak acid) and/or a base. In some embodiments, a fluid (e.g., a gastric fluid) may enter the reservoir such that it reacts with the acid and/or base to form a gas. In some embodiments, the reservoir may comprise a coating (e.g., such that the fluid does not contact the polymeric portion under the coating dissolves). In another exemplary embodiment, the jet injection component comprises a plunger/piston (e.g., activated by a spring associated with the plunger/piston) such that a material is expelled rapidly from the system.

In some embodiments, the tissue-interfacing component comprises a spring-actuated component. For example, a self-righting article comprising a tissue interfacing component (e.g., a needle) may be administered to a subject such that, the self-righting article orients at a location internal of the subject such that the tissue interfacing opponent punctures a tissue proximate the location internal of the subject. In some such amendments, an active pharmaceutical ingredient associated with the self-righting article may be released into and or proximate the tissue. In some embodiments, the tissue-interfacing component may penetrate the tissue. In some embodiments, the tissue is penetrated with a force of greater than or equal to 1 mN and less than or equal to 20,000 mN (e.g., greater than or equal to 10 mN and less than or equal to 20 mN, greater than or equal to 10 mN and less than or equal to 100 mN, greater than or equal to 100 mN and less than or equal to 20,000 mN, greater than or equal to 5,000 mN and less than or equal to 20,000 mN).

In some embodiments, the tissue interfacing component may be oriented within the self-righting article such that, upon administration to a subject, the tissue interfacing component is aligned substantially orthogonally (e.g., within 15° of orthogonal) with a tissue internal to the subject (e.g., GI mucosal tissue). In some embodiments, the tissue interfacing component may be disposed within a hollow portion of the self-righting device such that the tissue interfacing component releases (e.g., is triggered) from the self-righting device along a longitudinal axis of the hollow portion. For example, referring again to FIG. 1C, self-righting article may have a short longitudinal axis 105 aligned within 15 degrees of orthogonal of tissue engaging surface 125. In some embodiments, self-righting article has a longest longitudinal axis 110 that is perpendicular to a major axis of tissue interfacing component 120 (e.g., the short longitudinal axis 105 is parallel to the major axis of tissue interfacing component 120). In some embodiments, tissue interfacing component 120 is released (e.g., upon activation of the self-actuator) such that spring 130 expands along longitudinal axis 105 and/or tissue interfacing component travels parallel to the direction of longitudinal axis 105. In some such embodiments, tissue interfacing component may exit hole 103 and enter a tissue of the subject in a direction substantially parallel to longitudinal axis 105. In other embodiments, however, the tissue interfacing component is not aligned substantially orthogonally with a tissue internal to a subject.

In some embodiments, the self-righting article has a shortest longitudinal axis oriented within less than or equal to 15 degrees, less than or equal to 10 degrees, less than or equal to 5 degrees, less than or equal to 2 degrees, or less than or equal to 1 degree of vertical upon self-righting. In some embodiments, the self-righting article has a shortest longitudinal axis oriented within greater than or equal to 0.1 degrees, greater than or equal to 1 degree, greater than or equal to 2 degrees, greater than or equal to 5 degrees, or greater than or equal to 10 degrees. Combinations of the above-referenced ranges are also possible (e.g., greater than or equal to 0.1 degrees and less than or equal to 15 degrees). Other ranges are also possible.

In some embodiments, the tissue-interfacing component has a longest longitudinal axis oriented within less than or equal to 15 degrees, less than or equal to 10 degrees, less than or equal to 5 degrees, less than or equal to 2 degrees, or less than or equal to 1 degree of vertical upon self-righting. In some embodiments, the tissue-interfacing component has a longest longitudinal axis oriented within greater than or equal to 0.1 degrees, greater than or equal to 1 degree, greater than or equal to 2 degrees, greater than or equal to 5 degrees, or greater than or equal to 10 degrees. Combinations of the above-referenced ranges are also possible (e.g., greater than or equal to 0.1 degrees and less than or equal to 15 degrees). Other ranges are also possible.

In some embodiments, the reservoir is cylindrical in shape. Other shapes are also possible. For example, in some embodiments, the reservoir is rectangular in shape.

In an exemplary embodiment, the tissue-interfacing component comprises a plurality of microneedles. In another exemplary embodiment, the tissue interfacing component comprises a single needle. In yet another exemplary embodiment, the tissue interfacing component comprises a biopsy component (e.g., a biopsy jaw). In some embodiments, the tissue interfacing component may comprise an anchoring mechanism (e.g., a hook, a mucoadhesive). Tissue interfacing components are described in more detail, below.

In some embodiments, a first portion comprises a first material having a first average density. In some embodiments, the first material and/or the second material may be selected to impart a particular mass and/or density to the first portion and/or the second portion.

In some embodiments, the first material and/or second material is selected from the group consisting of polymers, ceramics, metals, and combinations thereof (e.g., metal filled polymer). In some embodiments, the first material and/or the second material may be biocompatible. In some embodiments, the metal may be selected from the group consisting of stainless steel, iron-carbon alloys, Field's metal, wolfram, molybdenum, gold, zinc, iron, and titanium.

In some embodiments, the ceramic may be selected from the group consisting of hydroxyapatite, aluminum oxide, calcium oxide, tricalcium phosphate, silicates, silicon dioxide, and zirconium oxide.

In some embodiments, the polymer may be selected from the group consisting of polycaprolactone, polylactic acid, polyethylene glycol, polypropylene, polyethylene, polycarbonate, polystyrene, and polyether ether ketone, and polyvinyl alcohol.

In an exemplary embodiment, the first material comprises a metal and the second material comprises a polymer.

The self-righting article generally has a geometric center (e.g., center of the geometric volume). In some embodiments, the density, mass, and/or volume of the first portion and/or the second portion may be selected such that the self-righting article exhibits self-righting behavior. For example, in some embodiments, a center of mass of the self-righting article may be offset from the geometric center such that the article, suspended via an axis passing through the geometric center. For example, referring again to FIG. 1C, the center of mass may be offset of an axis passing through the point of contact with the external surface 220 and is parallel to the vector of the gravitational force.

In some embodiments, a center of mass of the self-righting article may be offset from the geometric center by about 0.1 to 2 mm. In some embodiments, the center of mass of the self-righting article may be offset from the geometric center by 0.35 to 1 mm.

In some embodiments, the self-righting article may have a particular cross-sectional dimension. In some embodiments, the average cross-sectional dimension of the self-righting article is greater than or equal to 5 mm, greater than or equal to 6 mm, greater than or equal to 7 mm, greater than or equal to 8 mm, greater than or equal to 9 mm, greater than or equal to 10 mm, greater than or equal to 11 mm, greater than or equal to 12 mm, greater than or equal to 13 mm, greater than or equal to 14 mm, or greater than or equal to 15 mm. In some embodiments, the average cross-sectional dimension is less than or equal to 15 mm, less than or equal to 14 mm, less than or equal to 13 mm, less than or equal to 12 mm, less than or equal to 11 mm, less than or equal to 10 mm, less than or equal to 9 mm, less than or equal to 8 mm, less than or equal to 7 mm, less than or equal to 6 mm, or less than or equal to 5 mm. Combinations are also possible in some embodiments. For example, in some embodiments, the average cross-sectional dimension is greater than or equal to 5 mm and less than or equal to 15 mm.

In some embodiments, the self-righting article may have an average overall length of between 10 mm and 20 mm. In some embodiments, the overall length is greater than or equal to 10 mm, greater than or equal to 11 mm, greater than or equal to 12 mm, greater than or equal to 13 mm, greater than or equal to 14 mm, greater than or equal to 15 mm, greater than or equal to 16 mm, greater than or equal to 17 mm, greater than or equal to 18 mm, greater than or equal to 19 mm, or greater than or equal to 20 mm. In some embodiments, the average cross-sectional dimension is less than or equal to 20 mm, less than or equal to 19 mm, less than or equal to 18 mm, less than or equal to 17 mm, less than or equal to 16 mm, less than or equal to 15 mm, less than or equal to 14 mm, less than or equal to 13 mm, less than or equal to 12 mm, less than or equal to 11 mm, or less than or equal to 10 mm. Combinations are also possible in some embodiments. For example, in some embodiments, the average cross-sectional dimension is greater than or equal to 10 mm and less than or equal to 20 mm.

In some embodiments, the self-righting article may be administered (e.g., orally) to a subject. In some such embodiments, the self-righting article may comprise one or more active pharmaceutical ingredients. In some embodiments, the active pharmaceutical ingredient is released at a location internal of the subject (e.g. within the G.I. tract).

In some embodiments, one or more sensors may be associated with the self-righting article. For example, in some embodiments, one or more sensors may be used to determine the location of the self-righting article (e.g., a location internal to a subject) and/or to trigger actuation of one or more tissue interfacing components associated with the self-righting article. Non-limiting examples of suitable sensors include pH, gas, light, GPS, Bluetooth, orientation, proximity, thermal, fluid, and others.

In some embodiments, one or more of the first portion and/or second portion may be magnetic.

Certain exemplary embodiments are related to a method of delivering a pharmaceutical agent to a location internal of a subject. According to some embodiments, the method comprises administering, to the subject, a capsule comprising an outer shell and a self-righting article, and orienting the self-righting article at the location internal of a subject such that the tissue interfacing component punctures a tissue proximate the location internal of the subject.

In some embodiments, when the self-righting article is oriented in a longitudinal orientation relative to an external surface, a center of mass of the self-righting article is offset from a first axis that is perpendicular to the tissue-engaging surface, wherein the first axis passes through the point of contact with the external surface 220 and is parallel to the vector of the gravitational force, and wherein the article rotates toward the center of mass until the tissue-engaging surface contacts the external surface.

In some embodiments, the drug-containing portion comprises a plurality of tissue interfacing components (e.g., microneedles).

In some embodiments, a first spring is configured to displace the tissue interfacing component and a second spring is configured to displace a liquid active pharmaceutical agent.

### Self-Actuating Mechanisms

In some embodiments, the articles, systems, and methods disclosed comprise one or more self-actuating mechanism. Self-actuating articles including, for example, self-actuating tissue interfacing components such as self-actuating needles, self-actuating anchoring mechanisms, and/or self-actuating biopsy punches, are generally provided. Advantageously, in some embodiments, the self-actuating articles described herein may be useful as a general platform for delivery of a wide variety of pharmaceutical drugs that are typically delivered via injection directly into tissue due to degradation in the GI tract. The self-actuating articles described herein may also be used to deliver sensors, electrical stimulation, anchor systems described herein to tissue, and/or take biopsies without the need for an endoscopy. In some embodiments, the article comprises a spring (e.g., a coil spring, wave springs, Belleville washers, a beam, a membrane, a material having particular mechanical recovery characteristics). Those of ordinary skill in the art would understand that the term spring is not intended to be limited and may include various types of mechanical springs (e.g., tension springs, compression springs, coil springs, leaf springs, torsion springs, spiral springs, Belleville springs, etc.) as well as any reversibly compressive material and/or component which, after releasing an applied compressive force on the material/component, the material/component substantially returns to an uncompressed length of the material/component under ambient conditions (e.g., within 40%, within 50%, within 60%, within 70%, within 80%, within 90%, within 95%, or any percentage in between, of the length of the material/component prior to compression).

In some embodiments, the term spring of the self-actuating article may be provided as, or further comprise, an expanding component. Those of ordinary skill in the art would understand the term extending component comprises reversibly and irreversibly compressive materials and are components which, upon stimulating and/or releasing a restraint on the expanding component, the expanding component extends in at least one direction (e.g., along its length). In some embodiments, the expanding component comprises a gaseous composition(s) for expanding the gaseous volume expanding component (e.g., a mixture of baking soda and vinegar).

In some embodiments, the spring and/or expanding component may extend in at least one direction via thermal expansion, swelling (e.g., due to fluid absorption), a gas driven process, a pneumatic process, a hydraulic process, an electrical motor, a magnetic mechanism, a torsional spring mechanism, a chemical gas generator, and/or an self-catalyzing reaction. In an exemplary set of embodiments, the spring and/or expanding component may extend in at least one direction upon exposure of the spring and/or expanding component to a fluid (e.g., gastrointestinal fluid).

In some embodiments, the spring and/or the expanding component may be activated (e.g., extended in at least one direction, returns to an uncompressed length of the component) by any suitable activation mechanism. Non-limiting examples of suitable activation mechanisms include release of a pressure difference, electrical timer, light sensor, color sensor, enzymatic sensor, capacitance, magnetism, activation by applied stress (e.g., shape memory materials), external activation (e.g., applied magnetic field, applied light, reaction with gastrointestinal fluid such as stomach acid), and combinations thereof. In an exemplary set of embodiments, the spring and/or expanding component are activated by interaction (e.g., reaction) with a gastrointestinal fluid.

In some embodiments, the activation mechanism displaces the tissue interfacing component by a particular distance (e.g., less than or equal to 10 mm, less than or equal to 8 mm, less than or equal to 6 mm, less than or equal to 4 mm, less than or equal to 2 mm) and/or with a particular force (e.g., greater than or equal to 0.1 N, greater than or equal to 0.3 N, greater than or equal to 0.5 N, greater than or equal to 1 N, greater than or equal to 1.5 N).

As illustrated in FIGS. 2 and 5, exemplary system 300 comprises a first self-actuator comprising latch 341 configured to receive a first end of hub 335 (opposite the end configured to receive needle 320). The first end of hub 335 is further configured to receive support material 340 (e.g., the trigger), such that when support material 340 is loaded within the first end of hub 335, the first end of hub 335 engages latch 341, thus anchoring first spring 330 in a first position. In some embodiments, the first position is when first spring 330 is under tension and needle 320 is inside first reservoir 315. Upon dissolution of support material 340 (e.g., isomalt), the first end of hub 335 disengages from latch 341 and the tension applied to first spring 330 drives the needle into a second position. In some embodiments, when in the second position side hole 385 of needle 320 is in fluidic communication with liquid reservoir 355 and first end 302 of needle 320 exits the device through second end 303 of septum 380 into a space exterior of the device.

In some embodiments, the second self-actuator comprises latch 309 (which is comprised within endcap 310 in FIG. 2) configured to receive first end of second hub 370 (opposite the end configured to contact plunger 365). In some embodiments, plunger 365 and/or second spring 360 is operably linked to second hub 370. In some embodiments, the first end of second hub 370 is further configured to receive support material 375 (e.g., a trigger such as isomalt), such that when support material 375 is loaded within the first end of second hub 370, the first end of second hub 370 engages latch 309, thus anchoring plunger 365 and/or second spring 360 in a first position. In some embodiments, when in the first position, second spring 360 is under load (e.g., compression) and plunger 365 is proximate endcap 310. Upon dissolution of support material 375 (e.g., isomalt), the first end of second hub 370 disengages from latch 309 and the load (e.g., compression) applied to second spring 360 causes the plunger 365 to move in a horizontal direction that compresses liquid drug reservoir 355.

Similarly, FIGS. 3 and 6 illustrate self-actuation for exemplary system 400. In some embodiments, the first self-actuator comprises a latch. In some embodiments, the latch comprises a plurality of notches 446 located at a first end of hub 420. In some embodiments, the plurality of notches 446 are configured to engage support material 445, wherein the support material 445 is in contact with top 405. In some embodiments, when the plurality of notches 446 engage support material 445, the latch is engaged, thus anchoring first spring 430, second spring 435, third spring 440 in a first position. In some embodiments, the first position is when first spring 430, second spring 435, third spring 440 are under load (e.g., compression) and microneedle array 415 is inside first reservoir 415. Upon dissolution of support material 445 (e.g., isomalt), the latch becomes disengaged and the load (e.g., compression) applied to first spring 430, second spring 435, and/or third spring 440 drives the microneedle array 415 into a second position. In some embodiments, bottom housing 410 further comprises a plurality of holes 455, the plurality of holes 455 comprising a plug (e.g., a septum) such that, upon activation of the actuator (e.g., disengagement of the latch), the microneedle array 415 pierces the plug in hole 455 into a space exterior of the device. In some embodiments, the support material is a biodegradable coating. In some embodiments, the coating may have any suitable thickness. For example, the thickness of the coating may be greater than or equal to 3 mm, greater than or equal to 4 mm, or greater than or equal to 5 mm. In some embodiments, the thickness of the coating may be less than or equal to 6 mm, less than or equal to 5 mm, or less than or equal to 4 mm. Combinations of the above-referenced ranges are also possible (e.g., greater than or equal to 3 mm and less than or equal to 6 mm).

In some embodiments, the biodegradable coating at least partially degrades under physiological conditions. In some embodiments, the support material may be a brittle material. Non-limiting examples of suitable support materials include sugars, isomalt, and/or polymers (e.g., polyethylene glycol, polyvinylpyrrolidone, polyvinyl alcohol).

The support material may have any suitable cross-sectional dimension. In some embodiments, the average cross-sectional dimension of the support material is greater than or equal to 0.1 mm, greater than or equal to 0.5 mm, greater than or equal to 1 mm, greater than or equal to 2 mm, greater than or equal to 3 mm, greater than or equal to 4 mm, greater than or equal to 5 mm, greater than or equal to 6 mm, or greater than or equal to 10 mm. In some embodiments, the average cross-sectional dimension of the support material is less than or equal to 10 mm, less than or equal to 6 mm, less than or equal to 5 mm, less than or equal to 4 mm, less than or equal to 3 mm, less than or equal to 2 mm, less than or equal to 1 mm, less than or equal to 0.5 mm, or less than or equal to 0.1 mm. Combinations of the above-referenced ranges are also possible (e.g., greater than or equal to 0.1 mm and less than or equal to 10 mm). Other ranges are also possible.

In some embodiments, the support material, the spring, and/or the expanding component comprise one or more materials configured to dissolve (e.g., in an acidic environment in a pH neutral environment, in water, in a basic environment), melt at physiological temperature (e.g., 37° C.), change in stiffness (e.g., in response to a change in temperature, in response to fluid absorption), thermally expand, and/or change in shape (e.g., in response to fluid absorption, by deflation, by leakage).

Advantageously, the configuration and/or material used for the support material may permit tuning of the dissolution of the support material. In some embodiments, the dissolution of the support material may be tuned such that the tissue interfacing component is released from the article at a desired location and/or at a desired time.

The support material may comprise any suitable material. Non-limiting examples of suitable materials include sugars and derivatives thereof (e.g., sugar alcohols such as isomalt, sugar mixtures such as toffee), starch, calcium carbonate, zinc, sodium chloride, and/or polymers (e.g., polyethylene glycol, polyvinylpyrrolidone, polyvinyl alcohol, polyethylene oxide, diethyl pyrocarbonate, hydrogels). Other materials are also possible. Without wishing to be bound by theory, the support material may be selected to be relatively brittle (e.g., such that the spring is released upon dissolution of the support material). In some embodiments, the support material is selected from the group consisting of a sugar, a derivative of a sugar, starch, sugar alcohol, maltose, isomalt, calcium carbonate, zinc, sodium chloride, polymers, and combinations thereof. In some embodiments, the support material is isomalt.

In some embodiments, the support material may be configured to have a particular architecture which provides desirable dissolution profiles. For example, in some embodiments, the support material may be configured to enhance dissolution profiles, have controlled failure modes (e.g., breakage into small pieces at relatively predictable locations) and/or provide structural integrity of the support material.

In some embodiments, the support material has desirable mechanical properties (e.g., such that the spring recovers at least a portion of its uncompressed length relatively quickly). For example, In some embodiments, the support material may have a critical stress of greater than or equal to 0.01 N, greater than or equal to 0.1 N, greater than or equal to 0.5 N, greater than or equal to 1 N, greater than or equal to 2 N, greater than or equal to 3 N, greater than or equal to 5 N, greater than or equal to 7 N, greater than or equal to 10 N, greater than or equal to 15 N, greater than or equal to 20 N, greater than or equal to 25 N, greater than or equal to 30 N, greater than or equal to 35 N, greater than or equal to 40 N, greater than or equal to 45 N, greater than or equal to 50 N, or greater than or equal to 60 N, including any critical stress value in between. In some embodiments, the support material may have a critical stress of less than or equal to 70 N, less than or equal to 60 N, less than or equal to 50 N, less than or equal to 45 N, less than or equal to 40 N, less than or equal to 35 N, less than or equal to 30 N, less than or equal to 25 N, less than or equal to 20 N, less than or equal to 15 N, less than or equal to 10 N, less than or equal to 7 N, less than or equal to 5 N, less than or equal to 3 N, less than or equal to 2 N, less than or equal to 1 N, less than or equal to 0.5 N, or less than or equal to 0.1 N including any critical stress value in between. Combinations of the above-referenced ranges are also possible (e.g., greater than or equal to 10 N and less than or equal to 70 N, greater than or equal to 30 N and less than or equal to 45 N). Other ranges are also possible. The critical stress is generally the maximum force the support material can hold (e.g., as applied by the adjacent spring) before cracking and may be determined by calculating the critical stress, where:

$${σ}_{c}^{2} = \frac{2 γ E}{π a} ,$$

where σ_{c} is the critical stress applied by the spring, y is the surface energy of the material, E is the Young's modulus of the material, and a is the surface area perpendicular to the applied stress.

In some embodiments, the support material may have a characteristic dissolution time. In some embodiments, the characteristic dissolution time of the support material is less than or equal to 10 minutes, less than or equal to 9 minutes, less than or equal to 8 minutes, less than or equal to 7 minutes, less than or equal to 6 minutes, less than or equal to 5 minutes, less than or equal to 4 minutes, less than or equal to 3 minutes, or less than or equal to 2 minutes. In some embodiments, the characteristic dissolution time of the support material is greater than or equal to 1 minute, greater than or equal to 2 minutes, greater than or equal to 3 minutes, greater than or equal to 4 minutes, greater than or equal to 5 minutes, greater than or equal to 6 minutes, greater than or equal to 7 minutes, greater than or equal to 8 minutes, or greater than or equal to 9 minutes. Combinations of the above-referenced ranges are also possible (e.g., greater than or equal to 1 minute and less than or equal to 10 minutes). Other ranges are also possible. The characteristic dissolution time is determined as the time in which a support material begins to propagate a crack after exposure to gastrointestinal fluid.

### Spring and Spring Configurations

In some embodiments, articles, systems, and methods disclosed herein comprise one or more springs. Each of the one or more springs may have the following properties. For example, self-righting devices may comprise a first spring, a second spring, a third spring, a fourth spring, a fifth spring, and so forth. In some embodiments, at least one spring is operably linked to an actuation mechanism and/or a tissue interfacing component.

In some embodiments, the support material maintains at least a portion of the spring (e.g., first spring, second spring, etc.) under at least 5%, at least 10%, at least 15%, at least 20%, at least 25%, at least 30%, at least 40%, at least 50%, at least 60%, at least 70%, at least 80%, or at least 90% compressive strain under the first set of conditions. In some embodiments, the support material maintains at least a portion of the spring under less than or equal to 90%, less than or equal to 80%, less than or equal to 70%, less than or equal to 60%, less than or equal to 50%, less than or equal to 40%, less than or equal to 30%, less than or equal to 25%, less than or equal to 20%, less than or equal to 15%, or less than or equal to 10% compressive strain under the first set of conditions.

In some embodiments, the spring (e.g., first spring, second spring, etc.) recovers (e.g., within less than 10 minutes, less than 5 minutes, less than 1 minute, less than 30 seconds, less than 10 seconds, less than 5 seconds, less than 1 second, less than 0.1 seconds, less than 0.01 seconds) to a length of greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 85%, greater than or equal to 90%, greater than or equal to 95%, greater than or equal to 98%, or greater than or equal to 99% of the length of the spring (e.g., an uncompressed spring length) prior to applying and/or in the absence of the compressive strain (e.g., by the support material), including any percentage in between 10% and 99%. In some embodiments, the spring recovers to a length of less than or equal to 100%, less than or equal to 99%, less than or equal to 98%, less than or equal to 95%, less than or equal to 90%, less than or equal to 85%, less than or equal to 80%, less than or equal to 75%, less than or equal to 70%, less than or equal to 60%, less than or equal to 50%, less than or equal to 40%, less than or equal to 30%, or less than or equal to 20% of the length of the spring prior to applying and/or in the absence of the compressive strain, including any percentage in between 20% and 100%.

Advantageously, the use of springs and support materials as described herein may enable, for example, the release of a tissue interfacing component (e.g., a needle) associated with (e.g., operably linked with) the spring such that the tissue interfacing component contacts and/or penetrates tissue proximate the article. In an illustrative example, in some embodiments, a needle associated with the spring is administered to a subject such that, upon degradation of the support material, the spring recovers and the needle is pushed into tissue proximate the article such that the needle penetrates the tissue (e.g., a GI mucosal layer). In some such embodiments, an active pharmaceutical ingredient may be delivered into the tissue by the tissue interfacing components. For example, in some embodiments, the article comprises an active pharmaceutical ingredient such that, upon release of the spring at a location internal of a subject, the active pharmaceutical ingredient is released (e.g., into tissue proximate the location internal of the subject). In other embodiments, a biopsy may be conducted (e.g., by the tissue interfacing component such as a biopsy device) upon release of the spring by the support material.

In some embodiments, the tissue interfacing component comprises a needle, a fluidic channel, a patch or an array of needles (e.g., microneedles), a biopsy component, a hook, a mucoadhesive patch, or combinations thereof.

In some embodiments, the spring (e.g., first spring, second spring, etc.) comprises an elastic material. In some embodiments, the spring comprises a material selected from the group consisting of nitinol, metals, polymers, and combinations thereof. In some embodiments, the first spring and the second spring may be different in respect to at least one or more of the following: material, length, spring type, spring constant, and cross-sectional dimension. However, in some embodiments, the first spring and the second spring may be substantially the same.

In some embodiments, the spring (e.g., first spring, second spring, etc.) may have a particular spring constant. For example, in some embodiments, the spring constant of the spring may be greater than or equal to 100 N/m, greater than or equal to 150 N/m, greater than or equal to 200 N/m, greater than or equal to 250 N/m, greater than or equal to 300 N/m, greater than or equal to 350 N/m, greater than or equal to 400 N/m, greater than or equal to 450 N/m, greater than or equal to 500 N/m, greater than or equal to 600 N/m, greater than or equal to 700 N/m, greater than or equal to 800 N/m, greater than or equal to 900 N/m, greater than or equal to 1000 N/m, greater than or equal to 1100 N/m, greater than or equal to 1200 N/m, greater than or equal to 1300 N/m, or greater than or equal to 1400 N/m, less than or equal to 1500 N/m, less than or equal to 1800 N/m, greater than or equal to 2000 N/m, or greater than or equal to 2200 N/m and including any spring constant in between these values. In some embodiments, the spring constant of the spring may be less than or equal to 2200 N/m, less than or equal to 2000 N/m, less than or equal to 1800 N/m, less than or equal to 1500 N/m, less than or equal to 1400 N/m, less than or equal to 1300 N/m, less than or equal to 1200 N/m, less than or equal to 1100 N/m, less than or equal to 1000 N/m, less than or equal to 900 N/m, less than or equal to 800 N/m, less than or equal to 700 N/m, less than or equal to 600 N/m, less than or equal to 500 N/m, less than or equal to 450 N/m, less than or equal to 400 N/m, less than or equal to 350 N/m, less than or equal to 300 N/m, less than or equal to 250 N/m, less than or equal to 200 N/m, or less than or equal to 150 N/m, including any spring constant in between these values. Combinations of the above-referenced ranges are also possible (e.g., greater than or equal to 100 N/m and less than or equal to 500 N/m, greater than or equal to 100 N/m and less than or equal to 1500 N/m). Other ranges are also possible.

In some embodiments, the spring (e.g., first spring, second spring, etc.) is compressed (e.g., by the support material) by greater than or equal to 1 mm, greater than or equal to 2 mm, greater than or equal to 3 mm, greater than or equal to 4 mm, greater than or equal to 5 mm, greater than or equal to 6 mm, greater than or equal to 7 mm, greater than or equal to 8 mm, greater than or equal to 9 mm, greater than or equal to 10 mm, greater than or equal to 12 mm, greater than or equal to 15 mm, or greater than or equal to 20 mm along a longitudinal axis of the spring as compared to the uncompressed length of the spring. In some embodiments, the spring is compress by less than or equal to 20 mm, less than or equal to 15 mm, less than or equal to 12 mm, less than or equal to 10 mm, less than or equal to 9 mm, less than or equal to 8 mm, less than or equal to 7 mm, less than or equal to 6 mm, less than or equal to 5 mm, less than or equal to 4 mm, less than or equal to 3 mm, or less than or equal to 2 mm along a longitudinal axis of the spring as compared to the uncompressed length of the spring. Combinations of the above-referenced ranges are also possible (e.g., greater than or equal to 1 mm and less than or equal to 5 mm, greater than or equal to 5 mm and less than or equal to 10 mm). Other ranges are also possible.

In some embodiments, the spring (e.g., first spring, second spring, etc.) is configured to release a desirable amount of a stored compressive energy of the spring (e.g., upon exposure of the support material to a fluid such as gastrointestinal fluid). For example, the spring and/or the support material may be exposed to a fluid and, upon at least partial dissolution of the support material, the spring at least partially releases stored compressive energy e.g., to displace the tissue interfacing component operably linked to the spring (e.g., to release it into a tissue located internal to a subject). For example, in some embodiments, the spring is configured to release at least 10%, at least 20%, at least 30%, at least 40%, at least 50%, at least 60%, at least 70%, or at least 80% of the stored compressive energy of the spring, including any percentage in between these values. In some embodiments, the spring is configured to release at least 90% of the stored compressive energy of the spring, at least 92% of the stored compressive energy of the spring, at least 94% of the stored compressive energy of the spring, at least 96% of the stored compressive energy of the spring, at least 98% of the stored compressive energy of the spring, or at least 99% of the stored compressive energy of the spring (e.g., upon exposure of the support material to a fluid such as gastrointestinal fluid), including any percentage in between these values. In some embodiments, the spring is configured to release less than or equal to 100% of the stored compressive energy of the spring, less than 99% of the stored compressive energy of the spring, less than 98% of the stored compressive energy of the spring, less than 96% of the stored compressive energy of the spring, less than 94% of the stored compressive energy of the spring, less than 92% of the stored compressive energy of the spring, or less than 91% of the stored compressive energy of the spring. In some embodiments, the spring is configured to release less than or equal to 90%, less than or equal to 80%, less than or equal to 70%, less than or equal to 60%, less than or equal to 50%, less than or equal to 40%, less than or equal to 30%, or less than or equal to 20% of the stored compressive energy of the spring (e.g., upon exposure of the support material to a fluid such as gastrointestinal fluid), including any percentage in between these values. Combinations of the above-referenced ranges are also possible (e.g., at least 92% and less than 98% of the stored compressive energy of the spring, at least 94% and less than 96% of the stored compressive energy of the spring, at least 10% and less than or equal to 99%). Other ranges are also possible.

In some embodiments, the spring (e.g., first spring, second spring, etc.) is configured to release the stored compressive energy of the spring within any suitable time of exposing the support material to a fluid and/or mechanical failure (e.g., cracking, fracture) of the support material. For example, in some embodiments, the spring is configured to release the stored compressive energy (e.g., at least 10% of the stored compressive energy) of the spring within less than 5 ms, less than 4 ms, less than 3 ms, less than 2 ms, less than 1 ms, less than 0.5 ms, or less than 0.2 ms of mechanical failure of the support material. In some embodiments, the spring is configured to release the stored compressive energy of the spring within in greater than 0.1 ms, greater than 0.2 ms, greater than 0.5 ms, greater than 1 ms, greater than 2 ms, greater than 3 ms, or greater than 4 ms of mechanical failure of the support material. Combinations of the above-referenced ranges are also possible (e.g., within less than 5 ms and greater than 1 ms, within less than 2 ms and greater than 0.1 ms). Other ranges are also possible.

In some embodiments, the spring (e.g., first spring, second spring, etc.) is configured to release the stored compressive energy of the spring (e.g., at least 10% of the stored compressive energy) as described herein within less than 10 min, less than 9 min, less than 7 min, less than 5 min, less than 3 min, or less than 1 min of exposing the support material to a fluid, including any time in between these values. In some embodiments, the spring is configured to release the stored compressive energy of the spring within greater than 30 seconds, greater than 1 min, greater than 3 min, greater than 5 min, greater than 7 min, or greater than 9 min, including any time in between these values. Combinations of the above-referenced ranges (e.g., within less than 10 min and greater than 30 seconds, within less than 7 min and greater than 5 min). Other ranges are also possible.

Any combination of the above-referenced ranges are also possible. For example, in some embodiments, the spring is configured to release at least 10% (e.g., at least 90%) of the stored compressive energy of the spring within 10 min of exposing the support material to a fluid. In some embodiments, the spring is configured to release at least 10% (e.g., at least 90%) of a stored compressive energy of the spring within 30 seconds of exposing the support material to a fluid. In some embodiments, the spring is configured to release less than or equal to 100% of a stored compressive energy of the spring within 10 min of exposing the support material to a fluid. In some embodiments, the spring is configured to release less than or equal to 100% of the stored compressive energy of the spring within 30 seconds of exposing the support material to a fluid.

In some embodiments, the spring (e.g., first spring, second spring, etc.) is configured to release at least 10% (e.g., at least 90%) of the stored compressive energy of the spring within 5 ms of mechanical failure of the support material. In some embodiments, the spring is configured to release at least 10% (e.g., at least 90%) of a stored compressive energy of the spring within 0.1 ms of mechanical failure of the support material. In some embodiments, the spring is configured to release less than or equal to 100% of a stored compressive energy of the spring within 5 ms of mechanical failure of the support material. In some embodiments, the spring is configured to release less than or equal to 100% of the stored compressive energy of the spring within 0.1 ms of mechanical failure of the support material.

The spring (e.g., first spring, second spring, etc.) may have any suitable cross-sectional dimension. In some embodiments, the largest cross-sectional dimension of the (uncompressed) spring is greater than or equal to 1 mm, greater than or equal to 2 mm, greater than or equal to 3 mm, greater than or equal to 4 mm, greater than or equal to 5 mm, greater than or equal to 6 mm, or greater than or equal to 10 mm. In some embodiments, the largest cross-sectional dimension of the (uncompressed) spring is less than or equal to 10 mm, less than or equal to 6 mm, less than or equal to 5 mm, less than or equal to 4 mm, less than or equal to 3 mm, or less than or equal to 2 mm. Combinations of the above-referenced ranges are also possible (e.g., greater than or equal to 1 mm and less than or equal to 10 mm). Other ranges are also possible.

In some embodiments, the tissue interfacing component comprises a needle and the tissue is penetrated with a force of greater than or equal to 1 mN and less than or equal to 100 mN (e.g., greater than or equal to 10 mN and less than or equal to 20 mN). In some embodiments, the tissue interfacing component comprises a plurality of microneedles and the tissue is penetrated with a force of greater than or equal to 100 mN and less than or equal to 10 N (e.g., greater than or equal to 1 N and less than or equal to 2 N, greater than or equal to 100 mN and less than or equal to 6 N).

In some embodiments, and as described herein, the article may be oriented such that a longitudinal axis of the tissue interfacing component is orthogonal (e.g., within less than or equal to 10%, less than or equal to 5%, or less than or equal to 1% of) 90° to the tissue located proximate the article. In some embodiments, the self-actuating articles (e.g., comprising a tissue-interfacing component) described herein may be associated with one or more self-righting articles.

In an exemplary embodiment, the article comprises an outer shell, a spring at least partially encapsulated within the outer shell, a support material associated with the spring such that the support material maintains at least a portion of the spring under at least 5% compressive strain under ambient conditions, and a tissue interfacing component operably linked to the spring. In some embodiments, the article comprises a tissue interfacing component and a spring associated with the tissue interfacing component, the spring maintained in an at least partially compressed state by a support material under at least 5% compressive strain. According to some embodiments, the spring is configured to release at least 10% (e.g., at least 90%) of a stored compressive energy of the spring within 0.1 ms of mechanical failure of the support material. According to some embodiments, the article compresses a pharmaceutical agent associated with the tissue interfacing component. In some embodiments, the article comprises a self-righting article associated with the tissue interfacing component.

### Trigger/Support

In some embodiments, the articles, systems, and methods disclosed comprise one or more trigger/support material. According to exemplary embodiments described herein, a trigger of a drug delivery device may be configured to actuate the drug delivery device in the GI tract of a subject under a predetermined condition. In some embodiments, the predetermined condition includes one or more of a predetermined time after ingestion of the drug delivery device, a predetermined location in the GI tract, physical contact with the GI tract, physical manipulation in the GI tract (e.g., compression via peristalsis), one or more characteristics of the GI tract (e.g., pH, pressure, acidity, temperature, etc.), or combinations thereof. In some embodiments, the trigger may be a passive component configured to interact with the environment of the GI tract to actuate the drug delivery device. For example, in some embodiments the trigger may be a sugar plug, or other dissolvable material, configured to dissolve in the GI tract. The dissolvable plug may have a certain thickness and/or shape that at least partly determines the speed at which the sugar plug dissolves and ultimately actuates the drug delivery device. In some embodiments, the trigger has an oval shape, an egg shape, a spherical shape, an elliptical shape, a cylindrical shape, a conical shape, or a spherocylindrical shape. In another embodiment, the trigger may be at least partially formed by an enteric coating. For example, in some embodiments, a trigger may include both a sugar plug and an enteric coating, as the present disclosure is not so limited. Other appropriate materials for a dissolvable trigger may include, but are not limited to, sugar alcohols, such as disaccharides (e.g. Isomalt), water soluble polymers, such as Poly-vinyl alcohol, enteric coatings, time-dependent coatings, enteric and time-dependent coatings, temperature-dependent coatings, light-dependent coatings, and/or any other appropriate material capable of being dissolved within the GI tract of a subject. In some embodiments, a trigger may include a triggerable membrane including EDTA, glutathione, or another suitable chemical. In some embodiments, a sugar alcohol trigger may be employed in combination with an enteric coating configured to protect the sugar alcohol trigger until the drug delivery device is received in the GI tract of a subject. In some embodiments, the trigger may include a pH responsive coating to assist with delaying triggering until after ingestion. In some embodiments, the trigger may be a sensor that detects one or more characteristics of the GI tract. For example, a sensor detecting contact with a GI mucosal lining may be used to actuate the device. In embodiments where a sensor is employed, the trigger may also include an active component that moves in response to a predetermined condition being detected by the sensor. For example, a gate may be moved when contact with a GI mucosal tract is detected. In other embodiments the trigger may employ electrical power to melt or weaken a rupturable membrane (e.g., by applying a voltage across a conductive rupturable membrane) and/or trigger a chemical reaction. Of course, any suitable active or passive trigger may be employed for a drug delivery device, as the present disclosure is not so limited.

### Jetting

In some embodiments, an article, system, and methods disclosed herein comprises a jet injector system. Specific operating parameters which may be used to optimize a jet for delivery into different tissue locations may include, for example, jet power, diameter, dosage, standoff distance, fluid viscosity, fluid density, and other appropriate parameters as elaborated on further below.

In some embodiments, the systems comprise a rupturable (i.e., frangible) membrane that seals a reservoir containing an API. The rupturable membrane may be physically supported by a trigger such as a sugar plug, an enteric coating, or other dissolvable trigger configured to be dissolved in the GI tract after a predetermined time period and/or at a predetermined location of the GI tract while located in vivo. When the trigger activates, the membrane may rupture to release a controlled micro-jet of the API into the GI mucosal lining. The inventors have recognized the particular benefits of an abrupt transition between upstream and downstream fluid pathways relative to the rupturable membrane. More specifically, in some embodiments, the inventors have recognized the benefits of a flow path having a first maximum transverse dimension (e.g., first maximum diameter) downstream of the rupturable membrane that is larger than a second maximum transverse dimension (e.g., second maximum diameter) of the flow path upstream of the rupturable membrane. Such an arrangement may facilitate the membrane rupturing in a center portion of the membrane rather than at an edge location adjacent to a stress concentration. Without wishing to be bound by theory, providing such a functionality may help ensure a consistent reproducible jet having appropriate penetration characteristics to deliver the API to the GI mucosal lining.

In some embodiments, a drug delivery device configured for administration to a subject includes a reservoir configured to contain an API, a potential energy source, and a trigger configured to actuate at a predetermined location within the subject. The reservoir may be a liquid reservoir and may contain a solution containing the API in a predetermined dosage. The potential energy source may store energy inside of the drug delivery device to be released when the trigger is actuated. The drug delivery device may also include an outlet in fluid communication with the reservoir, and a rupturable membrane positioned along a flow path extending between the reservoir and the outlet. The rupturable membrane may seal the reservoir from the outlet, so that the API is contained inside of the reservoir until the membrane is ruptured. The potential energy source may pressurize or otherwise compress the reservoir to apply a pressure to the rupturable membrane. When the trigger is actuated, the rupturable membrane may rupture to allow the API to flow through the flow path and out of the outlet. The flow path of the drug delivery device may have a first maximum transverse dimension (e.g., first maximum diameter) downstream of the rupturable membrane that is larger than a second maximum transverse dimension (e.g., second maximum diameter) of the flow path upstream of the rupturable membrane so that the pressure applied to the membrane is applied in a center portion of the membrane that is removed from an outer edge of the membrane. In some embodiments, the rupturable membrane may define a transition between the first diameter and the second diameter, such that the flow path has non-continuous walls and the flow path steps from the first diameter to the second diameter at the rupturable membrane. While according to exemplary embodiments described herein a drug delivery device may generate an incompressible liquid jet, in other embodiments a jet generated by a drug delivery device may be formed of gases, viscous fluids, aerosolized powders, and/or other appropriate materials, as the present disclosure is not so limited.

In the context of the invention, a jet means a collimated flow of gas, viscous fluids, aerosolized powders, and/or other appropriate materials.

According to exemplary embodiments described herein, a rupturable membrane of a drug delivery device may be formed of a frangible material that is configured to rupture or otherwise break when an unsupported threshold pressure is applied to the membrane. In some embodiments, the rupturable membrane comprises one or more materials chosen from a metal, a polymeric material, an elastomeric material, a plastic material, a ceramic material, a composite material, and/or any other appropriate frangible or brittle material for delivering an active API to a subject. In some embodiments, the rupturable membrane comprises a metal foil, an elastomeric film, a rigid plastic film, a flexible plastic film, and/or any other appropriate material capable of rupturing when a threshold pressure for a given membrane is applied. In some embodiments, the metal is aluminum. In some embodiments, the metal is a biodegradable metal, such as iron. In some embodiments, the plastic material is a reinforced plastic material. In some embodiments, the composite material comprises carbon fibers. In some embodiments, a material for a rupturable membrane may be selected to promote a brittle failure of the membrane during actuation. The rupturable membrane may be configured to rupture in such a manner that a jet of fluid is formed by the drug delivery device. In some embodiments, a rupturable membrane may be configured to function as a nozzle defining a jet exiting a drug delivery device. Of course, a rupturable membrane may be formed of any suitable material configured to rupture when a trigger of the drug delivery device actuates, as the present disclosure is not so limited.

According to exemplary embodiments described herein, a rupturable membrane may have a thickness less than or equal to 150 μm, 100 μm, 75 μm, 50 μm, 25 μm, 15 μm, and/or any other appropriate thickness. Correspondingly, a rupturable membrane may have a thickness greater than or equal to 10 μm, 15 μm, 25 μm, 50 μm, 75 μm, and/or any other appropriate thickness. Combinations of the above-noted ranges are contemplated, including thicknesses between 10 μm and 25 μm, between 10 μm and 50 μm, between 10 μm and 75 μm, between 10 μm and 100 μm, between 10 μm and 150 μm, between 15 μm and 25 μm, between 15 μm and 50 μm, between 15 μm and 75 μm, between 15 μm and 100 μm, between 15 μm and 150 μm, between 25 μm and 50 μm, between 25 μm and 100 μm, between 25 μm and 150 μm, between 25 μm and 75 μm, between 50 μm and 75 μm, between 50 μm and 100 μm, between 50 μm and 150 μm, between 75 μm and 100 μm, between 75 μm and 150 μm, or between 100 μm and 150 μm. As described herein, the phrase “between one value and another value” includes the endpoints and all values between the endpoints.

According to exemplary embodiments described herein, a drug delivery device includes a potential energy source which is used to store energy in the drug delivery device that is used to generate a jet of an API when the drug delivery device is actuated. In some embodiment, the potential energy source may be a compressed gas. The compressed gas may be directly stored in the drug delivery device, or the compressed gas may be generated via a chemical reaction or phase change. For example, in some embodiments, dry ice may be stored in a reservoir of the drug delivery device so that compressed gas is generated as the dry ice sublimates. Alternatively, a compressed gas may be provided to a desired reservoir prior to sealing a drug delivery device. In some embodiments, the potential energy source may be a spring (e.g., a compressed compression spring). In some embodiments, the potential energy source may be a reaction reservoir. For example, the reaction reservoir may allow an acid and base to be combined to generate gas, leading to the expulsion of API from the drug delivery device when the trigger is actuated. Alternatively, in another embodiment, a trigger may detonate an explosive material located within a reservoir to generate pressurized gas for expelling the API from the drug delivery device. Of course, any suitable reaction or other potential energy source may be employed to pressurize and drive an API in a jet when a drug delivery device is actuated, as the present disclosure is not so limited.

Without wishing to be bound by theory, jetting power may be tuned to deliver an API into different target tissues within the GI tract with different penetration characteristics. Jetting power may be at least partly determined by jet velocity, fluid density, and jet diameter. Accordingly, a drug delivery device according to exemplary embodiments described herein may be appropriately sized and include an appropriate amount of potential energy to generate a jet with enough power to deliver an API to the GI mucosal lining in a desired location.

As noted above, jetting power of a drug delivery device may affect the efficacy of API delivery to a GI mucosal lining. Accordingly, a drug delivery device may employ an appropriate jetting power and velocity to penetrate a GI mucosal lining and deliver an API to a subject. In some embodiments, jetting power for a drug delivery device according to exemplary embodiments described herein is less than or equal to 250 W, 200 W, 150 W, 100 W, 75 W, 50 W, 20 W, 10 W, 5 W, 1 W, 100 mW, and/or any other suitable power. Correspondingly, a jetting power for a drug delivery device may be greater than or equal to 50 mW, 100 mW, 1 W, 5 W, 10 W, 20 W, 50 W, 75 W, 100 W, 150 W, 200 W, and/or any other suitable power. Combinations of the above noted ranges are contemplated, including jetting powers between 50 mW and 250 W, between 50 mW and 200 W, between 50 mW and 150 W, between 50 mW and 100 W, between 50 mW and 75 W, between 50 mW and 50 W, between 50 mW and 20 W, between 50 mW and 10 W, between 50 mW and 5 W, between 50 mW and 1 W, between 100 mW and 200 W, between 100 mW and 150 W, between 100 mW and 100 W, between 100 mW and 75 W, between 100 mW and 50 W, between 100 mW and 20 W, between 100 mW and 10 W, between 100 mW and 5 W, between 100 mW and 1 W, between 1 W and 200 W, between 1 W and 150 W, between 1 W and 100 W, between 1 W and 75 W, between 1 W and 50 W, between 1 W and 20 W, between 1 W and 10 W, between 1 W and 5 W, between 5 W and 200 W, between 5 W and 150 W, between 5 W and 100 W, between 5 W and 75 W, between 5 W and 50 W, between 5 W and 20 W, between 5 W and 10 W, between 10 W and 200 W, between 10 W and 150 W, between 10 W and 100 W, between 10 W and 75 W, between 10 W and 50 W, between 10 W and 20 W, between 20 W and 200 W, between 20 W and 150 W, between 20 W and 100 W, between 20 W and 75 W, between 20 W and 50 W, between 50 W and 200 W, between 50 W and 150 W, between 50 W and 100 W, between 50 W and 75 W, between 75 W and 200 W, between 75 W and 150 W, between 75 W and 100 W, between 75 W and 200 W, between 75 W and 150 W, between 75 W and 100 W, between 100 W and 200 W, between 100 W and 150 W, or between 150 W and 200 W. Of course, any jetting power suitable to deliver an API to a GI mucosal lining may be employed, as the present disclosure is not so limited.

To achieve the exemplary jetting powers described herein, a jet generated by a drug delivery device of exemplary embodiments described herein may have a corresponding velocity. Accordingly, in some embodiments, a drug delivery device may be configured to generate a jet having a velocity less than or equal to 250 m/s, 200 m/s, 150 m/s, 100 m/s, 75 m/s, 50 m/s, and/or another appropriate velocity. Correspondingly, a drug delivery device may be configured to generate a jet having a velocity greater than or equal to 20 m/s, 30 m/s, 50 m/s, 100 m/s, 150 m/s, 200 m/s, and/or another appropriate velocity. Combinations of the above-noted ranges are contemplated, including, but not limited to, jet velocities between 20 m/s and 250 m/s, between 20 m/s and 200 m/s, between 20 m/s and 100 m/s, between 20 m/s and 150 m/s, between 20 m/s and 100 m/s, between 20 m/s and 75 m/s, between 20 m/s and 50 m/s, between 50 m/s and 250 m/s, between 50 m/s and 200 m/s, between 50 m/s and 100 m/s, between 50 m/s and 150 m/s, between 50 m/s and 100 m/s, between 50 m/s and 75 m/s, between 75 m/s and 250 m/s, between 75 m/s and 200 m/s, between 75 m/s and 100 m/s, between 75 m/s and 150 m/s, between 75 m/s and 100 m/s, between 100 m/s and 250 m/s, between 100 m/s and 200 m/s, between 100 m/s and 150 m/s, between 150 m/s and 250 m/s, between 150 m/s and 200 m/s, or between 200 m/s and 250 m/s. Of course, any jet velocity suitable to deliver an API to a GI mucosal lining, or other structure associated with the GI tract of a subject, may be employed, as the present disclosure is not so limited.

In some embodiments, a maximum transverse dimension of an outlet and/or jet (e.g. a diameter) may be less than or equal to 1 mm, 800 μm, 600 μm, 350 μm, 300 μm, 250 μm, 200 μm, 150 μm, 100 μm, 75 μm, 50 μm, 25 μm, 10 μm, and/or any other appropriate dimension. Correspondingly, an outlet and/or jet maximum transverse dimension may be greater than or equal to 5 μm, 10 μm, 25 μm, 50 μm, 75 μm, 100 μm, 150 μm, 200 μm, 250 μm, 300 μm, 600 μm and/or any other appropriate dimension. Combinations of the above-noted ranges are contemplated, including, but not limited to, maximum transverse dimensions of a jet and/or outlet between 5 μm and 600 μm, between 10 μm and 600 μm, between 25 μm and 600 μm, between 50 μm and 600 μm, between 75 μm and 600 μm, between 100 μm and 600 μm, between 150 μm and 600 μm, between 200 μm and 600 μm, between 250 μm and 600 μm, between 300 μm and 600 μm, between 350 μm and 600 μm, between 5 μm and 350 μm, between 10 μm and 350 μm, between 25 μm and 350 μm, between 50 μm and 350 μm, between 75 μm and 350 μm, between 100 μm and 350 μm, between 150 μm and 350 μm, between 200 μm and 350 μm, between 250 μm and 350 μm, between 300 μm and 350 μm, between 5 μm and 300 μm, between 10 μm and 300 μm, between 25 μm and 300 μm, between 50 μm and 300 μm, between 75 μm and 300 μm, between 100 μm and 300 μm, between 150 μm and 300 μm, between 200 μm and 300 μm, between 250 μm and 300 μm, between 5 μm and 250 μm, between 10 μm and 250 μm, between 25 μm and 250 μm, between 50 μm and 250 μm, between 75 μm and 250 μm, between 100 μm and 250 μm, between 150 μm and 250 μm, between 200 μm and 250 μm, between 5 μm and 200 μm, between 10 μm and 200 μm, between 25 μm and 200 μm, between 50 μm and 200 μm, between 75 μm and 200 μm, between 100 μm and 200 μm, between 150 μm and 200 μm, between 5 μm and 150 μm, between 10 μm and 150 μm, between 25 μm and 150 μm, between 50 μm and 150 μm, between 75 μm and 150 μm, between 100 μm and 150 μm, between 5 μm and 100 μm, between 10 μm and 100 μm, between 25 μm and 100 μm, between 50 μm and 100 μm, between 75 μm and 100 μm, between 5 μm and 75 μm, between 10 μm and 75 μm, between 25 μm and 75 μm, between 50 μm and 75 μm, between 5 μm and 50 μm, between 10 μm and 50 μm, between 25 μm and 50 μm, between 5 μm and 25 μm, between 10 μm and 25 μm, or between 5 μm and 10 μm. Of course, any jet diameter suitable for delivery of an API to a GI mucosal lining may be employed, as the present disclosure is not so limited.

According to exemplary embodiments described herein, a drug delivery device includes a potential energy source configured to pressurize an API so that the API may be released in a jet into a GI tract mucosal lining. The pressure applied to the reservoir may affect jetting power and/or a jet velocity of an API jet emitted by the drug delivery device. In some embodiments, the potential energy source may apply a pressure to an API reservoir less than or equal to 1000 bar, 800 bar, 600 bar, 500 bar, 250 bar, 100 bar, 60 bar, 40 bar, 10 bar, 1 bar, and/or any other appropriate pressure. Correspondingly, the potential energy source may apply a pressure to an API reservoir greater than or equal to 0.1 bar, 1 bar, 10 bar, 40 bar, 60 bar, 100 bar, 250 bar, 500 bar, 600 bar, 800 bar, and/or any other appropriate pressure. Combinations of the above-noted ranged are contemplated, including, but not limited to, pressures between 0.1 bar and 1000 bar, between 0.1 bar and 800 bar, between 0.1 bar and 600 bar, between 0.1 bar and 500 bar, between 0.1 bar and 250 bar, between 0.1 bar and 100 bar, between 0.1 bar and 60 bar, between 0.1 bar and 40 bar, between 0.1 bar and 10 bar, between 0.1 bar and 1 bar, between 1 bar and 1000 bar, between 1 bar and 800 bar, between 1 bar and 600 bar, between 1 bar and 500 bar, between 1 bar and 250 bar, between 1 bar and 100 bar, between 1 bar and 60 bar, between 1 bar and 40 bar, between 1 bar and 10 bar, between 10 bar and 1000 bar, between 10 bar and 800 bar, between 10 bar and 600 bar, between 10 bar and 500 bar, between 10 bar and 250 bar, between 10 bar and 100 bar, between 10 bar and 60 bar, between 10 bar and 40 bar, between 10 bar and 800 bar, between 10 bar and 600 bar, between 10 bar and 500 bar, between 10 bar and 250 bar, between 10 bar and 100 bar, between 10 bar and 60 bar, between 10 bar and 40 bar, between 40 bar and 800 bar, between 40 bar and 600 bar, between 40 bar and 500 bar, between 40 bar and 250 bar, between 40 bar and 100 bar, between 40 bar and 60 bar, between 60 bar and 800 bar, between 60 bar and 600 bar, between 60 bar and 500 bar, between 60 bar and 250 bar, between 60 bar and 100 bar, between 100 bar and 800 bar, between 100 bar and 600 bar, between 100 bar and 500 bar, between 100 bar and 250 bar, between 250 bar and 800 bar, between 250 bar and 600 bar, between 250 bar and 500 bar, between 500 bar and 800 bar, between 500 bar and 600 bar, or between 600 bar and 800 bar. Of course, any suitable pressure may be applied to an API reservoir, as the present disclosure is not so limited.

A drug delivery device of exemplary embodiments described herein may be configured to deliver a predetermined dose of an API to a subject. According to exemplary embodiments described herein, a drug delivery device may include an API reservoir volume less than or equal to 500 μL, 300 μL, 200 μL, 150 μL, 100 μL, 75 μL, 50 μL, 25 μL, 10 μL, and/or any other appropriate volume. Correspondingly, a drug delivery device may contain an API reservoir volume greater than or equal to 1 μL, 5 μL, 10 μL, 25 μL, 50 μL, 75 μL, 100 μL, 200 μL, 300 μL, and/or any other appropriate volume. Combinations of the above-noted volumes are contemplated, including, but not limited to, reservoir volumes between 1 μL and 500 μL, between 1 μL and 300 μL, between 1 μL and 200 μL, between 1 μL and 150 μL, between 1 μL and 100 μL, between 1 μL and 75 μL, between 1 μL and 50 μL, between 1 μL and 25 μL, between 1 μL and 10 μL, between 10 μL and 500 μL, between 10 μL and 300 μL, between 10 μL and 200 μL, between 10 μL and 150 μL, between 10 μL and 100 μL, between 10 μL and 75 μL, 10 μL and 50 μL, between 10 μL and 25 μL, between 25 μL and 500 μL, between 25 μL and 300 μL, between 25 μL and 200 μL, between 25 μL and 150 μL, between 25 μL and 100 μL, between 25 μL and 75 μL, between 25 μL and 50 μL, between 50 μL and 500 μL, between 50 μL and 300 μL, between 50 μL and 200 μL, between 50 μL and 150 μL, between 50 μL and 100 μL, between 50 μL and 75 μL, between 75 μL and 500 μL, between 75 μL and 300 μL, between 75 μL and 200 μL, between 75 μL and 150 μL, between 75 μL and 100 μL, between 100 μL and 500 μL, between 100 μL and 300 μL, between 100 μL and 200 μL, between 100 μL and 150 μL, between 150 μL and 500 μL, between 150 μL and 300 μL, between 150 μL and 200 μL, between 200 μL and 500 μL, between 200 μL and 300 μL, or between 300 μL and 500 μL. Of course, any suitable reservoir volume may be employed in a drug delivery device, as the present disclosure is not so limited.

In some embodiments, a drug delivery device is sized and shaped to be ingested by a subject. Accordingly, the drug delivery device may be appropriately small so that the drug delivery device may be easily swallowed and subsequently pass through the GI tract, including the esophagus and pyloric opening within the stomach. In some embodiments, a drug delivery device may include an overall length, such as a maximum dimension along a longitudinal axis of the device, that is less than or equal to 40 mm, 30 mm, 20 mm, 10 mm, 5 mm, and/or another appropriate length. Correspondingly, a drug delivery device may have an overall length greater than or equal to 3 mm, 5 mm, 10 mm, 20 mm, 25 mm, and/or another appropriate length. Combinations of the above-noted ranges are contemplated, including, but not limited to, overall lengths between 5 mm and 30 mm, between 10 mm and 30 mm, between 20 mm and 30 mm, between 25 mm and 30 mm, between 5 mm and 25 mm, between 10 mm and 25 mm, between 20 mm and 25 mm, between 5 mm and 20 mm, between 10 mm. In some embodiments, a drug delivery device may have a maximum external transverse dimension, such as a diameter or other dimension that may be perpendicular to the longitudinal axis, that is less than or equal to 11 mm, 10 mm, 7 mm, 5 mm, and/or another appropriate dimension. Correspondingly, a drug delivery device may have a maximum external transverse dimension greater than or equal to 3 mm, 5 mm, 7 mm, 9 mm, and/or another appropriate dimension. Combinations of the above-noted ranges are contemplated, including, but not limited to, maximum external transverse dimensions between 3 mm and 11 mm, between 3 mm and 10 mm, between 3 mm and 7 mm, between 3 mm and 5 mm, between 5 mm and 11 mm. In some embodiments, a drug delivery device may have an overall volume less than or equal to 3500 mm³, 3000 mm³, 2500 mm³, 2000 mm³, 1500 mm³, 1000 mm³, 750 mm³, 500 mm³, 250 mm³, 100 mm³, and/or any other appropriate volume. Corresponding, a drug delivery device may have an overall volume greater than or equal to 50 mm³, 100 mm³, 250 mm³, 500 mm³, 750 mm³, 1000 mm³, 1500 mm³, 2000 mm³, 2500 mm³, and/or any other appropriate volume. Combinations of the above-noted ranged are contemplated, including, but not limited to, volumes between 1000 mm³ and 3000 mm³, 1500 mm³ and 3000 mm³, 50 mm³ and 500 mm³, 50 mm³ and 100 mm³, as well as 2000 mm³ and 3000 mm³. Of course, any suitable overall length, maximum external transverse dimension, and volume for an ingestible delivery device may be employed, as the present disclosure is not so limited.

As noted above, in some embodiments, it is desirable to deliver an active pharmaceutical ingredient to a tissue of the stomach of a subject. Accordingly, an appropriate peak power may be selected to allow a jet to penetrate tissue of the stomach proximate to a drug delivery device disposed within a stomach of a subject. In some instances, the peak power may be selected to avoid perforating a muscularis layer of the stomach. In such an embodiment, a peak power of a jet oriented towards a surface of a subject's stomach may be greater than or equal to 9 W, 10 W, 12 W, 15 W, 20 W, 25 W, 50 W, 100 W, and/or any other appropriate power. Correspondingly, a peak power of the jet may be less than or equal to 130 W, 100 W, 50 W, 25 W, 21 W, 15 W, 12 W, and/or any other appropriate power. Combinations of the foregoing ranges are contemplated including a peak power between 9 W and 130 W, between 9 W and 100 W, between 9 W and 50 W, between 9 W and 25 W, between 9 W and 21 W, between 9 W and 15 W, between 9 W and 12 W, between 10 W and 130 W, between 10 W and 100 W, between 10 W and 50 W, between 10 W and 25 W, between 10 W, and 21 W, between 10 W and 15 W, between 12 W and 130 W, between 12 W and 100 W, between 12 W and 50 W, between 12 W and 25 W, between 12 W and 21 W, between 12 W and 15 W between 15 W and 130 W, between 15 W and 100 W, between 15 W and 50 W, between 15 W and 25 W, between 15 W and 21 W, between 20 W and 130 W, between 20 W and 100 W, between 20 W and 50 W, between 20 W and 21 W, between 25 W and 130 W, between 25 W and 100 W, between 25 W and 50 W, between 50 W and 130 W, between 50 W and 100 W, or between 100 W and 130 W. As described herein, the phrase “between one value and another value” includes the endpoints and all values between the endpoints. In some instances, the above powers may be appropriate for forming a depot in the submucosal tissue and/or muscularis layer of the stomach of a subject.

Alternatively, embodiments in which a drug delivery device is configured to provide a jet for intraluminal delivery where a majority of the active pharmaceutical ingredient is injected into the intraluminal space of the stomach are also contemplated. In such an embodiment, a peak power of the jet may be less than 9 W. Additionally, embodiments in which a drug delivery device is configured for intraperitoneal delivery where a majority of the active pharmaceutical ingredient is injected into the peritoneal space by perforating the muscularis layer of the stomach are so contemplated. In some embodiments, an intraperitoneal injection within the stomach may correspond to jets with peak powers greater than about 40 W.

As also noted above, in some embodiments, it is desirable to deliver an active pharmaceutical ingredient to a tissue of the small intestine of a subject. Accordingly, an appropriate peak power may be selected to allow a jet to penetrate tissue of the small intestine proximate to a drug delivery device disposed within a small intestine of a subject. In some instances, the peak power may be selected to avoid perforating a muscularis layer of the small intestine. In such an embodiment, a peak power of a jet oriented towards a surface of a subject's small intestine may be greater than or equal to 3.0 W, 3.1 W, 3.2 W, 3.3 W, 3.4 W, 3.5 W, 4.0 W, 4.5 W, 5 W, 5.5 W, 6.0 W and/or any other appropriate power. Correspondingly, a peak power of the jet may be less than or equal to 6.5 W, 6.4 W, 6.3 W, 6.2 W, 6.1 W, 6.0 W, 5.5 W, 5.0 W, 4.5 W, and/or any other appropriate power. Combinations of the foregoing ranges are contemplated including a peak power between 3.0 W and 6.5 W, between 3.0 W and 6.4 W, between 3.0 W and 6.3 W, between 3.0 W and 6.2 W, between 3.0 W and 6.1 W, between 3.0 W and 6.0 W, between 3.0 W and 5.5 W, between 3.0 W and 5.0 W, between 3.0 W and 4.5 W, between 3.1 W and 6.5 W, between 3.1 W and 6.4 W, between 3.1 W and 6.3 W, between 3.1 W and 6.2 W, between 3.1 W and 6.1 W, between 3.1 W and 6.0 W, between 3.1 W and 5.5 W, between 3.1 W and 5.0 W, between 3.1 W and 4.5 W, between 3.2 W and 6.5 W, between 3.2 W and 6.4 W, between 3.2 W and 6.3 W, between 3.2 W and 6.2 W, between 3.2 W and 6.1 W, between 3.2 W and 6.0 W, between 3.2 W and 5.5 W, between 3.2 W and 5.0 W, between 3.2 W and 4.5 W, between 3.3 W and 6.5 W, between 3.3 W and 6.4 W, between 3.3 W and 6.3 W, between 3.3 W and 6.2 W, between 3.3 W and 6.1 W, between 3.3 W and 6.0 W, between 3.3 W and 5.5 W, between 3.3 W and 5.0 W, between 3.3 W and 4.5 W, between 3.4 W and 6.5 W, between 3.4 W and 6.4 W, between 3.4 W and 6.3 W, between 3.4 W and 6.2 W, between 3.4 W and 6.1 W, between 3.4 W and 6.0 W, between 3.4 W and 5.5 W, between 3.4 W and 5.0 W, between 3.4 W and 4.5 W, between 3.5 W and 6.5 W, between 3.5 W and 6.4 W, between 3.5 W and 6.3 W, between 3.5 W and 6.2 W, between 3.5 W and 6.1 W, between 3.5 W and 6.0 W, between 3.5 W and 5.5 W, between 3.5 W and 5.0 W, between 3.5 W and 4.5 W, between 4.0 W and 6.5 W, between 4.0 W and 6.4 W, between 4.0 W and 6.3 W, between 4.0 W and 6.2 W, between 4.0 W and 6.1 W, between 4.0 W and 6.0 W, between 4.0 W and 5.5 W, between 4.0 W and 5.0 W, between 4.0 W and 4.5 W, between 4.5 W and 6.5 W, between 4.5 W and 6.4 W, between 4.5 W and 6.3 W, between 4.5 W and 6.2 W, between 4.5 W and 6.1 W, between 4.5 W and 6.0 W, between 4.5 W and 5.5 W, between 4.5 W and 5.0 W, between 5.0 W and 6.5 W, between 5.0 W and 6.4 W, between 5.0 W and 6.3 W, between 5.0 W and 6.2 W, between 5.0 W and 6.1 W, between 5.0 W and 6.0 W, between 5.0 W and 5.5 W, between 5.5 W and 6.5 W, between 5.5 W and 6.4 W, between 5.5 W and 6.3 W, between 5.5 W and 6.2 W, between 5.5 W and 6.1 W, between 5.5 W and 6.0 W, between 6.0 W and 6.5 W, between 6.0 W and 6.4 W, between 6.0 W and 6.3 W, between 6.0 W and 6.2 W, between 6.0 W and 6.1 W, and/or any other appropriate range of peak powers. In some embodiments, a jetting power between 3.5 W and 6.5 W or between 4.0 W and 6.5 W may be preferable, as those jetting powers may have higher injection efficiency than other jetting powers. In some instances, the above powers may be appropriate for forming a depot in the submucosal tissue and/or muscularis layer of the small intestine of a subject.

Alternatively, embodiments in which a drug delivery device is configured to provide a jet for intraluminal delivery where a majority of the active pharmaceutical ingredient is injected into the intraluminal space of the small intestine are also contemplated. In such an embodiment, a peak power of the jet may be less than 3.0 W. Additionally, embodiments in which a drug delivery device is configured for intraperitoneal delivery where a majority of the active pharmaceutical ingredient is injected into the peritoneal space by perforating the muscularis layer of the small intestine are also contemplated. In some embodiments, an intraperitoneal injection within the small intestine may correspond to jets with peak powers greater than about 6.5 W, 7.0 W, and/or any other appropriate power range. According to exemplary embodiments described herein, the drug delivery device is administered to a subject orally. In other embodiments, the drug delivery device may be administered, endoscopically, rectally, vaginally, nasally, or uretherally, as the present disclosure is not so limited. Additionally, in some cases a drug delivery device according to exemplary embodiments described herein may be implanted into an organ of a subject. For example, a drug delivery device may be implanted into the arm, brain, peritoneum, etc. of the subject.

In some embodiments, it may be desirable to orient an outlet of a jet towards a surface of the GI tract of a subject prior to actuating a delivery device to help ensure delivery of an API into the desired tissue. Accordingly, depending on the particular embodiment, a variety of different strategies may be employed. For example, various mucoadhesive, dissolvable hooks for attaching to tissue, mucosal contact sensors, and other methods of either maintaining a delivery device in contact with and/or determining when they delivery device is proximate to a desired tissue within a GI tract may be used. For example, various self-righting or self-orienting structures and/or methods described in WO 2018/213600 A1 can be employed by the drug delivery device in accordance with the present disclosure. WO 2018/213600 A1 is incorporated herein by reference in its entirety. Additionally, in some embodiments, multiple outlets and corresponding multiple jets located at different positions on an exterior of the delivery device may be used to increase the chance of one of the jets being oriented towards a tissue proximate to the delivery device. Of course, it should be understood that embodiments in which a delivery device does not include sensors for sensing contact with and/or a component for attaching to a mucosal lining of a subject are also contemplated.

As used herein, the term “active pharmaceutical ingredient” (also referred to as a “drug” or “therapeutic agent”) refers to an agent that is administered to a subject to treat a disease, disorder, or other clinically recognized condition, or for prophylactic purposes, and has a clinically significant effect on the body of the subject to treat, prevent, and/or diagnose the disease, disorder, or condition. The active pharmaceutical ingredient may be delivered to a subject in a quantity greater than a trace amount to affect a therapeutic response in the subject. In some embodiments, active pharmaceutical ingredients (APIs) can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of some embodiments are compounds or chemicals traditionally regarded as drugs, vaccines, and biopharmaceuticals. Certain such APIs may include molecules such as proteins, peptides, hormones, nucleic acids, gene constructs, etc., for use in therapeutic, diagnostic, and/or enhancement areas. In some embodiments, the API is a small molecule and/or a large molecule. Accordingly, it should be understood that the API's described herein are not limited to any particular type of API. Additionally, while according to exemplary embodiments described herein a drug delivery device may deliver an API in the form of an incompressible liquid jet, in other embodiments a jet including an API generated by a drug delivery device may be formed of gases, viscous fluids, aerosolized powders, and/or other appropriate materials, as the present disclosure is not so limited.

In some embodiments, a drug delivery device is in a first state representative of a state prior to actuation of the drug delivery device. In some embodiments, the drug delivery device comprises a housing containing a potential energy source configured as a compressed gas compartment and an active pharmaceutical ingredient (API) reservoir. The compressed gas compartment and reservoir are separated by a piston slidably received in an interior of the housing. The piston includes a piston seal configured to inhibit fluid transfer between the compressed gas compartment and the reservoir. The piston transfers pressure from the compressed gas compartment to the reservoir. That is, the compressed gas inside of the gas compartment pressurizes the API inside of the reservoir. In some embodiments, the reservoir is fluidly connected to a nozzle that reduces a maximum transverse dimension of a fluid flow path, which may include a downstream opening of the nozzle, from a transverse dimension of the reservoir to a maximum transverse dimension corresponding to a desired maximum transverse dimension (e.g. a diameter) of a jet. The nozzle is configured to generate a jet of API when the pressurized API flows out of the nozzle. The drug delivery device also includes a fluid outlet in fluid communication with the nozzle. In the depicted embodiment, a maximum transverse dimension of the outlet is greater than a maximum transverse dimension of a downstream opening formed in the nozzle. A rupturable membrane is positioned between the nozzle and the outlet. According to some embodiments, the rupturable membrane may be formed of a metal foil though other rupturable membranes may also be used. The rupturable membrane is configured to rupture under the pressure of the pressurized API in the reservoir. In some embodiments, the device includes a trigger in the form of a dissolvable plug physically retained within an outlet of the device where the plug is disposed against a surface of the membrane on a side of the membrane opposite the nozzle. The dissolvable plug may be configured to physically support the rupturable membrane prior to being dissolved. Accordingly, when the trigger is positioned in the outlet the rupturable membrane is supported and does not rupture under pressure of the API.

In some embodiments, a drug delivery device includes a diffusion barrier positioned between the gas compartment and the piston. The gas diffusion barrier may be configured to prevent diffusion of the gas contained in the gas compartment over time into the API reservoir. Additionally, the diffusion barrier may be configured to prevent diffusion of gas contained in the gas compartment over time out of the housing. The diffusion barrier may inhibit gas diffusion more so than the piston seal alone, such that the device is suitable for longer shelf storage life. The diffusion barrier may be a rupturable barrier configured to break when the rupturable membrane ruptures. In some embodiments, the diffusion barrier may be formed of a metal foil, though other materials may also be used. Like the rupturable membrane, the diffusion barrier may be configured to rupture under the pressure of the gas inside of the gas compartment. However, the piston and the underlying fluid in the API reservoir are configured to support the diffusion barrier. That is, the diffusion barrier is supported by the piston until the device is actuated and the rupturable membrane is ruptured. As the piston moves toward the nozzle to expel the API in the API reservoir, the support is removed from the diffusion barrier and the diffusion barrier breaks to allow the gas in the gas compartment to continue to apply pressure to the piston.

In some embodiments, when a trigger dissolves inside of a GI tract of the subject, the physical support for the rupturable membrane is removed. Accordingly, the pressure from the API through the nozzle applies an unsupported force to the rupturable membrane sufficient to rupture the rupturable membrane. Once ruptured, the API flows out of the reservoir in a jet from the nozzle and into GI tract mucosal tissue of a subject. In some embodiments, the jet may have an appropriate combination of power, velocity, and diameter to penetrate the tissue to a desired depth. Again, as the piston advances toward the nozzle, the diffusion barrier breaks under the pressure of the gas in the gas compartment.

In some embodiments, the nozzle and outlet are configured such that the rupturable membrane reliably ruptures in a center portion of the membrane to ensure consistent jetting performance. In some embodiments, the nozzle has an exit port flow path adjacent the membrane having a first diameter (e.g., first maximum transverse dimension). The outlet has a second diameter (e.g., second maximum transverse dimension) that is larger than the first diameter. Accordingly, a flow path upstream of the membrane has a diameter less than a diameter of a flow path downstream of the membrane, where the membrane defines a transition (i.e., a step) between the first diameter and second diameter. As a result, the pressure applied to the membrane is concentrated in a central portion of the membrane while the physical support is distributed over a larger area. Accordingly, even if the trigger dissolves unevenly the stress on the membrane is concentrated on a central portion of the membrane aligned with the nozzle. Thus, when the membrane bursts, the API has a free flow path out of the API reservoir to the outlet where the jet is directed through the outlet without contacting the walls of the outlet prior to impinging on the underlying tissue.

In some embodiments, the trigger is configured as a dissolvable plug prior to dissolution in the GI tract. The dissolvable plug occludes and is retained in an outlet of the drug delivery device and supports the rupturable membrane. An API reservoir containing pressurized API applies pressure to the membrane on a side of the membrane opposite the trigger. In particular, the API applies pressure through an opening of a nozzle of the drug delivery device to the rupturable membrane which may have a downstream opening proximate to the membrane and trigger with a maximum transverse dimension (i.e. diameter) that is smaller than a corresponding maximum transverse dimension of a fluidly connected reservoir.

In some embodiments, the trigger begins to dissolve in the GI tract. In some embodiments, the dissolvable plug is configured to dissolve more quickly in a center portion of the dissolvable plug (i.e., further from the walls of the outlet). Nevertheless, the dissolvable plug may dissolve unevenly relative to a longitudinal centerline of the dissolvable plug due to environmental factors in the GI tract as well as irregularities in the dissolvable plug. In some embodiments, the trigger dissolves to an extent where a center portion of the membrane is no longer physically supported by the trigger. Accordingly, the membrane bulges under the pressure of the API in the API reservoir. Continued dissolution of the trigger ultimately results in the bulge bursting and the API being released from the API reservoir in a jet through the nozzle.

The skilled artisan will understand that even when the dissolvable plug trigger dissolves unevenly the membrane may rupture in a center portion aligned with the nozzle. As discussed previously, this may be enabled by providing a nozzle having a first diameter smaller than a second diameter of the outlet. Accordingly, as the trigger dissolves quickest in a central portion of the dissolvable plug that is spaced away from the walls of the outlet, the bulge forms in a portion of the membrane aligned with the nozzle. The edges of the membrane remain supported, such that an edge of the membrane does not tear away, which could result in an inefficient or unsatisfactory jet of API. The stress concentration in the center portion of the rupturable membrane also assists in ensuring a clean rupture that does not interfere with the formation of a jet of API from the nozzle that is directed out of the outlet without impinging on the walls of the outlet.

In some embodiments, a dissolvable plug trigger is configured to dissolve more quickly in the center of the trigger. To achieve this result, the dissolvable trigger may be potted and polished so that its exterior face exposed to the surrounding environment is both concave and wetted to an outlet of the delivery device. The dissolvable trigger may also be inspected for bubbles, cracks, and incomplete melting, features which may affect the dissolution pattern of the dissolvable trigger. Of course, any suitable arrangement for a dissolvable trigger may be employed, as the present disclosure is not so limited.

It should be noted that in some embodiments, a drug delivery device may have a single nozzle, a single outlet, or a plurality of nozzles and/or outlets. In some embodiments, a rupturable membrane may be positioned in a flow path upstream of the plurality of nozzles and/or outlets. In some embodiments, including a plurality of outlets in a drug delivery device may improve success rate for penetration of GI tract tissue, as the multiple outlets may compensate for variations in drug delivery device orientation. However, additional outlets may sacrifice payload efficiency, as a single payload may be split between each of the outlets, where only a select few of the outlets actually deliver a dose to GI tract tissue. Accordingly, in some embodiments a drug delivery device may include two outlets to improve reliability in view of orientation variations without greatly diminishing payload efficiency. Of course, a drug delivery device may include any suitable number of outlets, as the present disclosure is not so limited.

As noted above, jetting power may be tuned to deliver an API into different target tissues within the GI tract with different penetration characteristics. Jetting power may be at least partly determined by jet velocity, fluid density, and jet diameter. Accordingly, a drug delivery device according to exemplary embodiments described herein may be appropriately sized and include an appropriate amount of potential energy to generate a jet with enough power to deliver an API into the tissue of the GI tract in a desired location.

In embodiments in which systems and/or methods are used to actuate delivery of an API when an outlet is oriented towards gastrointestinal tissue proximate to the drug delivery device, it may be desirable to maintain an orientation of the outlet and corresponding jet within a predetermined range of angles relative to the underlying tissue. This may help to provide a desired combination of force and/or power of the jet in a direction oriented perpendicular to a surface of the adjacent tissue. For example, an angle of a jet emitted from an outlet relative to a direction normal to the underlying tissue surface may be less than or equal to 20°, 15°, 10°, 5°, and/or any other appropriate range of angles including angles both greater than and less than those noted above. The above-noted angular relationship of the jet direction emitted from an outlet of a device versus a direction normal to the underlying tissue surface may be provided using any of the above noted methods and structures for actuating a delivery device while in a desired orientation relative to the underlying tissue.

In some embodiments, it may be desirable to form one or more components of a drug delivery device from a biocompatible and/or bio inert material. For example, various components may be exposed to the fluids and/or solids present within the gastrointestinal tract of a subject when ingested. Accordingly, components that may be exposed to the fluids and/or solids present within the gastrointestinal tract may be made from materials including, but not limited to: metals that are relatively inert to the gastrointestinal environment such as titanium; non-toxic and/or inert polymers such as polydimethylsiloxane (PDMS), polycaprolactone (PCL); salts; carbohydrates; and/or any other appropriate material for a desired application. In instances where a particular component used in a delivery device may be inappropriate for exposure to the environment within the gastrointestinal system of a subject, a non-reactive polymeric and/or metallic coating may be applied to the component to isolate the underlying material from the exterior environment. Alternatively, such a component may be contained within a portion of the delivery device that is not exposed to the exterior environment during operation. In view of the above, it should be understood that the various components disclosed herein may be made using any appropriate combination materials as the disclosure is not limited to any particular construction and/or combination of materials of the various components.

In some embodiments, the actuation mechanism comprises a self-actuating component comprising a first spring component and a first support material adapted to maintain the first spring component in at least a partially compressed state, wherein the first support material is configured for at least partial degradation in a biological fluid.

In some embodiments, the tissue interfacing component comprises a jet injection component.

In some embodiments, the first spring component is configured to expel liquid active pharmaceutical agent through a jet injection component at a penetration velocity allowing the liquid active pharmaceutical agent to penetrate gastric submucosa of a lumen wall.

### Tissue Interfacing Component

In some embodiments, as described above and herein, the articles, systems, and methods disclosed herein comprise a tissue interfacing component. In some embodiments, the tissue-interfacing component comprises a needle, a biopsy component, a projectile, a plurality of microneedles (e.g., a microneedle array), a hook, a mucoadhesive patch, or combinations thereof. In some embodiments, as described herein and above, the tissue interfacing component is configured to penetrate tissue (e.g., skin, tongue, tissue of the GI tract such as GI mucosal tissue).

In some embodiments, the tissue interfacing component comprising a solid therapeutic agent (e.g., a solid API) and a second material (e.g., a support(ing) material for the solid API such as a binder and/or a polymer) such that the solid therapeutic agent is present in the component in an amount of greater than or equal to 10 wt % versus the total weight of the tissue interfacing component. Such tissue-interfacing components may be useful for delivery of API doses (e.g., to a subject). Advantageously, in some embodiments, the reduction of volume required to deliver the required API dose as compared to a liquid formulation permits the creation of solid needle delivery systems (e.g., microneedle array 415) for a wide variety of drugs in a variety of places/tissues (e.g., tongue, GI mucosal tissue, skin) and/or reduces and/or eliminates the application of an external force in order to inject a drug solution through the small opening in the needle. In some embodiments, a physiologically relevant dose may be present in a single tissue interfacing component (e.g., having a relatively high API loading).

In some embodiments, the API is substantially solid (e.g., a powder, a compressed powder, a crystalline solid, an amorphous solid) i.e. a solid therapeutic agent. In some embodiments, the API may be in liquid form.

In some embodiments, the tissue in penetrated with a force of greater than or equal to 1 mN and less than or equal to 20 N (e.g., greater than or equal to 10 mN and less than or equal to 20 mN, greater than or equal to 1 mN and less than or equal to 100 mN, greater than or equal to 20 mN and less than or equal to 1 N, greater than or equal to 1 N and less than or equal to 20 N, greater than or equal to 10 N and less than or equal to 20 N).

In some embodiments, a tissue-interfacing component comprises a needle and/or a plurality of microneedles comprising a relatively high API loading (e.g., greater than or equal to 10 wt % versus the total weight of the component). Without wishing to be bound by theory, it is believed that such configurations may significantly reduce the number of needles and/or the overall size of the microneedle array required to deliver a particular API dose, as compared to traditional microneedles (e.g., generally comprising less than 10 wt % loading and/or requiring a plurality of microneedles on the order of thousands to tens of thousands of microneedles to deliver a similar dose).

In some embodiments, the tissue-interfacing component has a particular largest dimension (e.g., length). In some embodiments, the largest dimension of the tissue interfacing component is greater than or equal to 1 mm, greater than or equal to 2 mm, greater than or equal to 3 mm, greater than or equal to 5 mm, greater than or equal to 7 mm, greater than or equal to 10 mm, greater than or equal to 12 mm, greater than or equal to 15 mm, greater than or equal to 20 mm, greater than or equal to 25 mm, greater than or equal to 30 mm, or greater than or equal to 50 mm. In some embodiments, the largest dimension of the tissue interfacing component is less than or equal to 100 mm, less than or equal to 50 mm, less than or equal to 30 mm, less than or equal to 25 mm, less than or equal to 20 mm, less than or equal to 15 mm, less than or equal to 12 mm, less than or equal to 10 mm, less than or equal to 7 mm, less than or equal to 5 mm, less than or equal to 3 mm, or less than or equal to 2 mm. Combinations of the above-referenced ranges are also possible.

In some embodiments, the tissue-interfacing component (e.g., a needle and/or a microneedle array comprising a plurality of microneedles) has an average cross-sectional dimension (e.g., diameter of each individual microneedle) of greater than or equal to 0.25 mm, greater than or equal to 0.5 mm, greater than or equal to 0.6 mm, greater than or equal to 0.7 mm, greater than or equal to 0.8 mm, greater than or equal to 0.9 mm, greater than or equal to 1 mm, greater than or equal to 1.1 mm, greater than or equal to 1.2 mm, greater than or equal to 1.3 mm, greater than or equal to 1.4 mm, greater than or equal to 1.5 mm, greater than or equal to 1.7 mm, mm, greater than or equal to 1.9 mm, greater than or equal to 2.5 mm, greater than or equal to 3.0 mm, greater than or equal to 4.0 mm, or greater than or equal to 5.0 mm. In some embodiments, the tissue-interfacing component has an average cross-sectional dimension of less than or equal to 6.0 mm, less than or equal to 5.0 mm, less than or equal to 4.0 mm, less than or equal to 3.0 mm, less than or equal to 2.5 mm, less than or equal to 1.9 mm, less than or equal to 1.7 mm, less than or equal to 1.5 mm, less than or equal to 1.4 mm, less than or equal to 1.3 mm, less than or equal to 1.2 mm, less than or equal to 1.1 mm, less than or equal to 1 mm, less than or equal to 0.9 mm, less than or equal to 0.8 mm, less than or equal to 0.7 mm, or less than or equal to 0.6, or less than or equal to 0.5 mm. Combinations of the above-referenced ranges are also possible (e.g., greater than or equal to 0.5 mm and less than or equal to 2.0 mm). Other ranges are also possible.

In some embodiments, the tissue interfacing component may comprise a plurality of microneedles. In some such embodiments, the plurality of microneedles may have a particular base largest cross-sectional dimension (e.g., diameter of the base), a particular height, and/or a particular spacing.

In some embodiments, the average diameter of the base of the plurality of microneedles is greater than or equal to 100 microns, greater than or equal to 150 microns, greater than or equal to 200 microns, greater than or equal to 250 microns, greater than or equal to 300 microns, greater than or equal to 350 microns, greater than or equal to 400 microns, or greater than or equal to 450 microns. In some embodiments, the average diameter of the base of the plurality of microneedles is less than or equal to 500 microns, less than or equal to 450 microns, less than or equal to 400 microns, less than or equal to 350 microns, less than or equal to 300 microns, less than or equal to 250 microns, less than or equal to 200 microns, or less than or equal to 150 microns. Combinations of the above-referenced ranges are also possible (e.g., greater than or equal to 100 microns and less than or equal to 500 microns). Other ranges are also possible.

In some embodiments, the average height of the plurality of microneedles is greater than or equal to 0.1 mm, greater than or equal to 0.2 mm, greater than or equal to 0.5 mm, greater than or equal to 0.7 mm, greater than or equal to 1 mm, greater than or equal to 1.2 mm, greater than or equal to 1.5 mm, or greater than or equal to 2 mm. In some embodiments, the average height of the plurality of microneedles is less than or equal to 2.5 mm, less than or equal to 2 mm, less than or equal to 1.5 mm, less than or equal to 1.2 mm, less than or equal to 1 mm, less than or equal to 0.7 mm, less than or equal to 0.5 mm, or less than or equal to 0.2 mm. Combinations of the above-referenced ranges are also possible (e.g., greater than or equal to 0.1 mm and less than or equal to 2.5 mm). Other ranges are also possible.

In some embodiments, the average spacing (e.g., spacing between adjacent microneedles in the plurality of microneedles) of the plurality of microneedles may be greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1000 microns, greater than or equal to 1100 microns, greater than or equal to 1200 microns, greater than or equal to 1300 microns, or greater than or equal to 1400 microns. In some embodiments, the average spacing of the plurality of microneedles is less than or equal to 1500 microns, less than or equal to 1400 microns, less than or equal to 1300 microns, less than or equal to 1200 microns, less than or equal to 1100 microns, less than or equal to 1000 microns, less than or equal to 900 microns, less than or equal to 800 microns, less than or equal to 700 microns, less than or equal to 600 microns, less than or equal to 500 microns, less than or equal to 400 microns, less than or equal to 300 microns, or less than or equal to 200 microns. Combinations of the above-referenced ranges are also possible (e.g., greater than or equal to 100 microns and less than or equal to 1500 microns). Other ranges are also possible.

In some embodiments, a microneedle array comprises a plurality of microneedles, the plurality of microneedles comprising an array of rows and columns of microneedles. In some embodiments, the number of rows is greater than or equal to 1, greater than or equal to 2, greater than or equal to 3, greater than or equal to 4, greater than or equal to 5, greater than or equal to 10, greater than or equal to 15, greater than or equal to 20, greater than or equal to 50, or greater than or equal to 100. In some embodiments, the number of rows is less than or equal to 200, less than or equal to 100, less than or equal to 50, less than or equal to 20, less than or equal to 15, less than or equal to 10, less than or equal to 5, less than or equal to 4, less than or equal to 3, or less than or equal to 2. Combinations of the above-referenced ranges are also possible (e.g., greater than or equal to 1 row and less than or equal to 200 rows of microneedles).

In some embodiments, the number of columns is greater than or equal to 1, greater than or equal to 2, greater than or equal to 3, greater than or equal to 4, greater than or equal to 5, greater than or equal to 10, greater than or equal to 15, greater than or equal to 20, greater than or equal to 50, or greater than or equal to 100. In some embodiments, the number of columns is less than or equal to 200, less than or equal to 100, less than or equal to 50, less than or equal to 20, less than or equal to 15, less than or equal to 10, less than or equal to 5, less than or equal to 4, less than or equal to 3, or less than or equal to 2. Combinations of the above-referenced ranges are also possible (e.g., greater than or equal to 1 row and less than or equal to 200 columns of microneedles). As would be understood by those of ordinary skill in the art based upon the teachings of this specification, the number of rows and columns need not necessarily be equal (e.g., although in some embodiments, the number of rows and columns of microneedles may be equal).

As would also be understood by those of ordinary skill in the art based upon the teachings of this specification, the microneedles need not necessarily be in a rectangular array and may have any suitable pattern (e.g., a random array, an alternating array, an irregular array, a concentric array) or shape. For example, in some embodiments, the array may comprise offset rows and/or columns.

Each microneedle may have any suitable shape including, but not limited to, cylindrical, conical, pyramid, candle, spike, spear, square, pentagonal, hexagonal, octagonal and rocket shape. Each microneedle may be solid or hollow.

In some embodiments, the total number of microneedles in the array is greater than or equal to 2 or more microneedles, 5 or more microneedles, 10 or more microneedles, 20 or more microneedles, 30 or more microneedles, 40 or more microneedles, 50 or more microneedles, 100 or more microneedles, 200 or more microneedles, 500 or more microneedles, or 1000 or more microneedles. In some embodiments, the total number of microneedles in the array may be less than or equal to 1000 microneedles, less than or equal to 500 microneedles, less than or equal to 200 microneedles, less than or equal to 100 microneedles, less than or equal to 50 microneedles, less than or equal to 40 microneedles, less than or equal to 30 microneedles, less than or equal to 20 microneedles, or less than or equal to 10 microneedles. Combinations of the above-referenced ranges are also possible (e.g., greater than or equal to 2 microneedles and less than or equal to 1000 microneedles). Other ranges are also possible.

In an exemplary set of embodiments, the microneedle array comprises a 2×2 array of microneedles, a 1×10 array of microneedles, a 50×50 array of microneedles, or a 7×14 array of microneedles. Other arrays are also possible.

Advantageously, in some embodiments, the tissue-interfacing component (e.g., needle and/or microneedle array), dissolves relatively quickly, reducing and/or eliminating the risk of secondary penetration by the component in undesired locations. In some embodiments, the largest cross-sectional dimension (e.g., length) of the component is designed to be delivered to whichever organ it is targeting to prevent pain and/or undesired perforation of the GI tract.

In some embodiments, the tissue interfacing component comprises a microneedle array. For example, as illustrated in FIG. 3, tissue interfacing component 415 comprises 14 microneedles along a major longitudinal axis of system 400. In some embodiments, the plurality of microneedles comprise a mucoadhesive material. Non-limiting examples of suitable mucoadhesive materials include polymers such as poly(vinyl alcohol), hydroxylated methacrylate, and poly(methacrylic acid), polyacrylates (e.g., polyacrylic acid, thiolated poly(acrylic acid), Carbopol®), cyanoacrylates, sodium carboxymethylcellulose, hyaluronic acid, hydroxypropylcellulose, polycarbophil, chitosan, mucin, alginate, xanthan gum, gellan, poloxamer, cellulose acetophthalate, methyl cellulose, hydroxy ethyl cellulose, poly(amidoamine) dendrimers, poly(dimethyl siloxane), poly(vinyl pyrrolidone), polycarbophil, combinations thereof, and copolymers thereof.

In some embodiments, the plurality of microneedles comprises a solid therapeutic agent (e.g., API) and a second material (if present), such that the solid therapeutic agent is present in the tissue interfacing component (e.g., needle and/or plurality of microneedles) in an amount of greater than or equal to 10 wt % versus the total weight of the tissue interfacing component (e.g., needle and/or plurality of microneedles). In some embodiments, the solid therapeutic agent is present in the tissue interfacing component (e.g., plurality of microneedles) in an amount of greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, greater than or equal to 90 wt %, greater than or equal to 95 wt %, greater than or equal to 98 wt %, or greater than or equal to 99.1 wt % versus the total weight of the tissue interfacing component (e.g., plurality of microneedles). In some embodiments, the solid therapeutic agent is present in the tissue interfacing component (e.g., plurality of microneedles) in an amount of less than or equal to 100 wt %, less than or equal to 99 wt %, less than or equal to 98 wt %, less than or equal to 95 wt %, less than or equal to 90 wt %, less than or equal to 80 wt %, less than or equal to 70 wt %, less than or equal to 60 wt %, less than or equal to 50 wt %, less than or equal to 40 wt %, less than or equal to 30 wt %, or less than or equal to 20 wt % versus the total weight of the tissue interfacing component (e.g., plurality of microneedles). Combinations of the above-referenced ranges are also possible (e.g., greater than or equal to 10 wt % and less than or equal to 100 wt %, greater than or equal to 80 wt % and less than or equal to 100 wt %). Other ranges are also possible. In an exemplary set of embodiments, the solid therapeutic agent is present in the tissue interfacing component (e.g., plurality of microneedles) in an amount greater than or equal to 80 wt % and less than or equal to 100 wt % versus the total weight of the tissue interfacing component.

In some embodiments, the solid therapeutic agent is present in the plurality of microneedles in an amount of greater than or equal to 0 wt %, greater than or equal to 5 wt %, greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, greater than or equal to 90 wt %, greater than or equal to 95 wt %, greater than or equal to 98 wt %, or greater than or equal to 99 wt % versus the total weight of the plurality of microneedles. In some embodiments, the solid therapeutic agent is present in the plurality of microneedles in an amount of less than or equal to 100 wt %, less than or equal to 99 wt %, less than or equal to 98 wt %, less than or equal to 95 wt %, less than or equal to 90 wt %, less than or equal to 80 wt %, less than or equal to 70 wt %, less than or equal to 60 wt %, less than or equal to 50 wt %, less than or equal to 40 wt %, less than or equal to 30 wt %, less than or equal to 20 wt %, less than or equal to 10 wt %, or less than or equal to 5 wt % versus the total weight of the plurality of microneedles. Combinations of the above-referenced ranges are also possible (e.g., greater than or equal to 10 wt % and less than or equal to 100 wt %, greater than or equal to 80 wt % and less than or equal to 100 wt %). Other ranges are also possible. In an exemplary embodiment, the plurality of microneedles substantially comprises only the solid therapeutic agent.

In some embodiments, the solid therapeutic agent is present in a tip of a plurality of microneedles in an amount of greater than or equal to 0 wt %, greater than or equal to 5 wt %, greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, greater than or equal to 90 wt %, greater than or equal to 95 wt %, greater than or equal to 98 wt %, or greater than or equal to 99 wt % versus the total weight of the tip of a plurality of microneedles. In some embodiments, the solid therapeutic agent is present in the tip of a plurality of microneedles in an amount of less than or equal to 100 wt %, less than or equal to 99 wt %, less than or equal to 98 wt %, less than or equal to 95 wt %, less than or equal to 90 wt %, less than or equal to 80 wt %, less than or equal to 70 wt %, less than or equal to 60 wt %, less than or equal to 50 wt %, less than or equal to 40 wt %, less than or equal to 30 wt %, less than or equal to 20 wt %, less than or equal to 10 wt %, or less than or equal to 5 wt % versus the total weight of the tip of a plurality of microneedles. Combinations of the above-referenced ranges are also possible (e.g., greater than or equal to 10 wt % and less than or equal to 100 wt %, greater than or equal to 80 wt % and less than or equal to 100 wt %). Other ranges are also possible. In an exemplary embodiment, the tip of a plurality of microneedles substantially comprises only the solid therapeutic agent. In another exemplary embodiment, the tip of a plurality of microneedles substantially comprises no solid therapeutic agent.

In some embodiments, the tissue interfacing component comprises greater than or equal to 10 wt % (e.g., greater than or equal to 80 wt %) solid therapeutic agent, regardless of the makeup of the plurality of microneedles, versus the total weight of the tissue interfacing component.

In some embodiments, the tissue interfacing component comprises greater than or equal to 0.1 mg, greater than or equal to 0.5 mg, greater than or equal to 0.8 mg, greater than or equal to 1 mg, greater than or equal to 1.5 mg, greater than or equal to 2 mg, greater than or equal to 2.5 mg, greater than or equal to 3 mg, greater than or equal to 4 mg, greater than or equal to 5 mg, greater than or equal to 7 mg, greater than or equal to 9 mg of therapeutic agent (e.g., solid therapeutic agent). In some embodiments, the tissue interfacing component comprises less than or equal to 10 mg, less than or equal to 9 mg, less than or equal to 7 mg, less than or equal to 5 mg, less than or equal to 4 mg, less than or equal to 3 mg, less than or equal to 2.5 mg, less than or equal to 2 mg, less than or equal to 1.5 mg, less than or equal to 1 mg, less than or equal to 0.8 mg, less than or equal to 0.5 mg, or less than or equal to 0.2 mg of therapeutic agent. Combinations of the above-referenced ranges are also possible (e.g., greater than or equal to 0.1 mg and less than or equal to 10 mg). Other ranges are also possible.

In some embodiments, the plurality of microneedles comprises a base portion and a tip portion. In some embodiments, at least a portion of the solid therapeutic agent (e.g., API) is associated with a base portion and/or one or more tips of the tissue interfacing component. For example, in some embodiments, the solid therapeutic agent and second material (if present) are distributed substantially homogeneously in the tissue interfacing component (e.g., in the base portion and/or in the tip). In some embodiments, the solid therapeutic agent may be a coating (e.g., disposed on at least a portion of the tip(s)) such that the tissue interfacing component comprises greater than or equal to 10 wt % solid therapeutic agent versus the total weight of the tissue interfacing component.

In some embodiments, the tissue interfacing component may comprise an additional coating. In some embodiments, the additional coating may comprise a material configured to e.g., slow the dissolution time relative to the dissolution of the tissue interfacing component without said additional coating. Non-limiting examples of suitable additional coating materials including Zn, Al, Mg, polymers (e.g., enteric polymers, polycaprolactone, parylene, hypromellose, polyethylene glycol), and combinations thereof. Other additional coating materials are also possible. In some embodiments, the additional coating may be configured such that the solid therapeutic agent is released over a particular amount of time. For example, in some embodiments, the additional coating is configured such that the solid therapeutic agent is released in less than or equal to 6 months, less than or equal to 3 months, less than or equal to 1 month, less than or equal to 2 weeks, less than or equal to 1 week, less than or equal to 4 days, less than or equal to 2 days, less than or equal to 1 day, less than or equal to 12 hours, less than or equal to 6 hours, less than or equal to 3 hours, less than or equal to 1 hour, less than or equal to 30 minutes, less than or equal to 15 minutes, less than or equal to 10 minutes, less than or equal to 5 minutes, or less than or equal to 2 minutes (e.g., upon exposure of the additional coating to a fluid such as gastric fluid). In some embodiments, the additional coating is configured such that the solid therapeutic agent is released in greater than or equal to 1 minute, greater than or equal to 2 minutes, greater than or equal to 5 minutes, greater than or equal to 10 minutes, greater than or equal to 15 minutes, greater than or equal to 30 minutes, greater than or equal to 1 hour, greater than or equal to 3 hours, greater than or equal to 6 hours, greater than or equal to 12 hours, greater than or equal to 1 day, greater than or equal to 2 days, greater than or equal to 4 days, greater than or equal to 1 week, greater than or equal to 2 weeks, greater than or equal to 1 month, or greater than or equal to 3 months. Combinations of the above-referenced ranges are also possible (e.g., greater than or equal to 1 minute and less than or equal to 1 day, greater than or equal to 1 day and less than or equal to 2 weeks, greater than or equal to 1 week and less than or equal to 6 months). Other ranges are also possible.

In some embodiments, the tissue interfacing component comprises a plurality of microneedles comprising the solid therapeutic agent and the second material (if present).

In some embodiments, at least a portion of the solid therapeutic agent is present on at least a surface of the tip. In some embodiments, at least a portion of the second material is present on at least a surface of the tip.

In some embodiments, a tissue interfacing component comprises an inlet and an outlet, and a channel fluidically connected to the inlet and the outlet (e.g., the inlet is in fluidic communication with the outlet). In some embodiments, the interfacing component comprises more than one inlet and/or more than one outlet. In some embodiments, the interfacing component comprises greater than or equal to 1, greater than or equal to 2, greater than or equal to 3, greater than or equal to 4, greater than or equal to 5, greater than or equal to 6, greater than or equal to 7, greater than or equal to 8, greater than or equal to 9, or greater than or equal to 10 inlets. In some embodiments, the interfacing component comprises less than or equal to 10, less than or equal to 9, less than or equal to 8, less than or equal to 7, less than or equal 6, less than or equal to 5, less than or equal to 4, less than or equal 3, less than or equal to 2, or less than or equal to 1 inlet. Combinations are also possible (e.g., interfacing component comprising more than 1 inlet and less than 10 inlets).

In some embodiments, the interfacing component comprises greater than or equal to 1, greater than or equal to 2, greater than or equal to 3, greater than or equal to 4, greater than or equal to 5, greater than or equal to 6, greater than or equal to 7, greater than or equal to 8, greater than or equal to 9, or greater than or equal to 10 outlets. In some embodiments, the interfacing component comprises less than or equal to 10, less than or equal to 9, less than or equal to 8, less than or equal to 7, less than or equal 6, less than or equal to 5, less than or equal to 4, less than or equal 3, less than or equal to 2, or less than or equal to 1 outlets. Combinations are also possible (e.g., interfacing component comprising more than 1 outlet and less than 10 outlets).

The tissue-interfacing components described herein may be formed using any suitable method. In some embodiments, the tissue-interfacing component is formed by providing the solid therapeutic agent and the second material (if present) and centrifuging and/or compressing, using at least 1 MPa of pressure, the solid therapeutic agent and a second material together to form the tissue interfacing component. In some embodiments, the second material (if present) and the solid therapeutic agent is heated to form the tissue interfacing component.

In some embodiments, the tissue-interfacing component is formed using at least 1 MPa of pressure, at least 2 MPa of pressure, at least 3 MPa of pressure, at least 5 MPa of pressure, at least 7 MPa of pressure, at least 10 MPa of pressure, at least 12 MPa of pressure, at least 15 MPa of pressure, at least 20 MPa of pressure, at least 25 MPa of pressure, at least 30 MPa of pressure, at least 40 MPa of pressure, at least 50 MPa of pressure, at least 75 MPa of pressure, at least 150 MPa of pressure, at least 300 MPa of pressure, at least 600 MPa of pressure, at least 900 MPa of pressure, at least 1 GPa of pressure, or at least 1.2 GPa of pressure. In some embodiments, the tissue-interfacing component is formed using less than or equal to 1.4 GPa of pressure, less than or equal to 1.2 GPa of pressure, less than or equal to 1 GPa of pressure, less than or equal to 900 MPa of pressure, less than or equal to 600 MPa of pressure, less than or equal to 300 MPa of pressure, less than or equal to 150 MPa of pressure, less than or equal to 100 MPa of pressure, less than or equal to 75 MPa of pressure, less than or equal to 50 MPa of pressure, less than or equal to 40 MPa of pressure, less than or equal to 30 MPa of pressure, less than or equal to 25 MPa of pressure, less than or equal to 20 MPa of pressure, less than or equal to 15 MPa of pressure, less than or equal to 12 MPa of pressure, less than or equal to 10 MPa of pressure, less than or equal to 7 MPa of pressure, less than or equal to 5 MPa pressure, less than or equal to 3 MPa of pressure, or less than or equal to 2 MPa of pressure. Combinations of the above-referenced ranges are also possible (e.g., at least 1 MPa of pressure and less than or equal to 100 MPa of pressure, at least 20 MPa of pressure and less than or equal to 100 MPa of pressure, at least 100 MPa and less than or equal to 1.4 GPa of pressure). Other ranges are also possible.

In some embodiments, the tissue interfacing component may be formed at a particular temperature. For example, the tissue interfacing component, in some embodiments, is formed at a temperature of greater than or equal to 50° C., greater than or equal to 60° C., greater than or equal to 70° C., greater than or equal to 80° C., greater than or equal to 90° C., greater than or equal to 100° C., or greater than or equal to 120° C. In some embodiments, the tissue interfacing component is formed at a temperature of less than or equal to 150° C., less than or equal to 130° C., less than or equal to 120° C., less than or equal to 110° C., less than or equal to 100° C., less than or equal to 90° C., less than or equal to 80° C., less than or equal to 70° C., or less than or equal to 60° C. Combinations of the above referenced ranges are also possible (e.g., greater than or equal to 50° C. and less than or equal to 130° C.). Other temperatures and ranges are also possible.

Advantageously, the tissue interfacing component may have desirable mechanical properties (e.g., Young's elastic modulus) e.g., such that the tissue interfacing component may suitably puncture tissue of the gastrointestinal tract. In some embodiments, the Young's elastic modulus of the tissue interfacing component is greater than or equal to 100 MPa (e.g., greater than or equal to 125 MPa, greater than or equal to 150 MPa, greater than or equal to 175 MPa, greater than or equal to 200 MPa, greater than or equal to 250 MPa, greater than or equal to 300 MPa, or greater than or equal to 350 MPa). In some embodiments, the tissue interfacing component has a Young's elastic modulus of less than or equal to 400 MPa, less than or equal to 350 MPa, less than or equal to 300 MPa, less than or equal to 250 MPa, less than or equal to 200 MPa, less than or equal to 175 MPa, less than or equal to 150 MPa, or less than or equal to 125 MPa. Combinations of the above-referenced ranges are also possible (e.g., greater than or equal to 100 MPa and less than or equal to 250 MPa, greater than or equal to 100 MPa and less than or equal to 400 MPa). Other ranges are also possible.

In some embodiments, the tissue interfacing component may be configured to penetrate a particular depth into human gastrointestinal mucosal tissue at a particular force. For example, the tissue interfacing component may be configured to penetrate greater than or equal to 1 mm (e.g., greater than or equal to 2 mm, greater than or equal to 3 mm, or greater than or equal to 4 mm) with a force of less than or equal to 20 N (e.g., less than or equal to less than or equal to 10 N, less than or equal to 5 N, less than or equal to 1 N, less than or equal to 500 mN, less than or equal to 100 mN, less than or equal to 50 mN, less than or equal to 20 mN, less than or equal to 15 mN, less than or equal to 10 mN, less than or equal to 5 mN).

In some embodiments, the second material comprises a polymerizable monomer and/or a polymer. In some embodiments, the second material is biodegradable. Non-limiting examples of suitable materials for the second material include polyethylene glycol, polyvinylpyrrolidone, polylactic acid, polysaccharides (e.g., maltose, lactose, starch, cellulose), acacia, methyl cellulose, gelatin, tragacanth, clays, HPMC, stearic acid, sodium stearate, magnesium stearate, talc, polyethylene glycol, mineral oil, preservatives (e.g., phenol, paraben, cetrimide), antioxidants (e.g., gallic acid, tocopherol), derivatives thereof, and combinations thereof.

In some embodiments, the tissue interfacing component comprises a coating having a yield strength of greater than or equal to 50 MPa (e.g., greater than or equal to 60 MPa, greater than or equal to 70 MPa, or greater than or equal to 80 MPa).

In some embodiments, the coating may be comprised of a thin film metal, a ceramic or a Diamond Like Coating (DLC). In some embodiments, the tissue interfacing component does not comprise a coating.

In some embodiments, the coating may be comprised of a corrodible material (e.g. iron, zinc, aluminum or alloys) such that when the coating comes in contact with the physiological environment it will disintegrate and present the therapeutic agent. In some embodiments, the coating may comprise a polymer such as parylene, as described herein.

In some embodiments, the tissue interfacing component may be configured to deliver a particular amount of active pharmaceutical agent per square centimeter of tissue of a subject. For example, in some embodiments, the tissue interfacing component is configured to deliver greater than or equal to 0.01 μg, greater than or equal to 0.05 μg, greater than or equal to 0.1 μg, greater than or equal to 0.2 μg, greater than or equal to 0.5 μg, greater than or equal to 0.7 μg, greater than or equal to 1 μg, greater than or equal to 2 μg, greater than or equal to 5 μg, or greater than or equal to 10 μg of pharmaceutical agent per square centimeter of tissue of the subject proximate the penetration location of the tissue interfacing component. In some embodiments, the tissue interfacing component is configured to deliver less than or equal to 20 μg, less than or equal to 5 μg, less than or equal to 2 μg, less than or equal to 1 μg, less than or equal to 0.7 μg, less than or equal to 0.5 μg, less than or equal to 0.2 μg, less than or equal to 0.1 μg, or less than or equal to 0.05 μg of pharmaceutical agent per square centimeter of tissue. Combinations of the above-referenced ranges are also possible (e.g., greater than or equal to 1 μg and less than or equal to 20 μg). In some embodiments, the tissue interfacing component is configured to deliver greater than or equal to 1 μg of pharmaceutical agent per square centimeter of tissue of the subject over any suitable time period (e.g., in greater than or equal to 0.1 seconds, in greater than or equal to 0.5 seconds, in greater than or equal to 1 second, in greater than or equal to 5 seconds, in greater than or equal to 30 seconds, greater than or equal to 1 minute, greater than or equal to 5 minutes, 10 minutes, greater than or equal to 30 minutes, greater than or equal to 1 hour, greater than or equal to 4 hours, greater than or equal to 24 hours, greater than or equal to 48 hours, greater than or equal to 72 hours, greater than or equal to 96 hours, greater than or equal to 120 hours, greater than or equal to 144 hours, greater than or equal to 168 hours).

In some embodiments, the tissue interfacing component comprises a binder (e.g., In some embodiments, the second material is a binder). Non-limiting examples of suitable binders include sugar such as sorbitol and sucrose, gelatin, polymers such as polyvinyl alcohol (PVA), polyethylene glycol (PEG), polycaprolactone (PCL), and polyvinylpyrrolidone (PVP), and polymers comprising ethanol or other Class 3 organic solvents (e.g., acetic acid, heptane, acetone, formic acid, isobutyl acetate, etc.).

In an exemplary embodiment, the tissue interfacing component (e.g., microneedle array comprising a plurality of microneedles) comprises greater than or equal to 80 wt % solid active pharmaceutical agent versus the total article weight. In some embodiments, the article comprises greater than or equal to 1 mg of active pharmaceutical agent.

Some exemplary embodiments are related to a method of forming the tissue interfacing component (e.g., microfluidic array), wherein the method comprises introducing, into a mold, a composition comprising greater than 80 wt % solid pharmaceutical agent versus the total weight of the composition, applying greater than or equal to 1 MPa of pressure to the composition, and heating the composition to a temperature of at least 70° C. for at least 1 minute.

According to some embodiments, the tissue interfacing component has a Young's elastic modulus of greater than or equal to 100 MPa. In some embodiments, the tissue interfacing component is configured to penetrate at least 1 mm into human gastrointestinal mucosal tissue with a force of less than or equal to 20 mN. According to some embodiments, the tissue interfacing component is configured to deliver at least 1 mg of pharmaceutical agent per square centimeter of a tissue of a subject, and/or the tissue interfacing component comprises greater than or equal to 1 mg of active pharmaceutical agent per square centimeter. In some embodiments, the devices, article, and methods disclosed herein are configured to deliver at least 1 mg of pharmaceutical agent per square centimeter of a tissue of a subject, and/or greater than or equal to 1 mg of active pharmaceutical agent per square centimeter of tissue in a subject.

### Agents

According to some embodiments, the composition and methods described herein are compatible with one or more therapeutic, diagnostic, and/or enhancement agents, such as drugs, nutrients, microorganisms, in vivo sensors, and tracers. In some embodiments, the active substance is a therapeutic, nutraceutical, prophylactic or diagnostic agent. While much of the specification describes the use of therapeutic agents, other agents listed herein are also possible.

According to some embodiments, the pharmaceutical agent comprises is selected from the group consisting of bacteriophage, DNA, mRNA, insulin, human growth hormone, monoclonal antibodies, adalimumab, epinephrine, and ondansetron. In some exemplary embodiments, the active pharmaceutical agent is cast into a mold to form the article. In some embodiments, the mold is centrifuged.

In some embodiments, the agent comprises a drug encapsulated within a particle, such as a nanoparticle (e.g., a lipid nanoparticle, LNP). In some embodiments, the pharmaceutical agent comprises an mRNA encapsulated within an LNP.

Agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of some embodiments are compounds or chemicals traditionally regarded as drugs, vaccines, and biopharmaceuticals, Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti (retro) viral agents like entecavir, dolutegravir, rilpivirine, and cabotegravir, antibiotics like doxycycline, ciprofloxacin, and azithromycin, anti-malarial agents, and Synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In some embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In some embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferative, such as anti-cancer agents, antihistamines, antimigraine drugs, hormones, prostaglandins, antimicrobials (including antibiotics, antifungals, antivirals, antiparasitic), antimuscarinics, anxiolytics, bacteriostatic, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergic, electrolytes, gastro-intestinal drugs, muscle relaxants, nutritional agents, vitamins, parasympathomimetic, stimulants, anorectics and anti-narcoleptics. Nutraceuticals can also be incorporated into the drug delivery device. These may be vitamins, supplements such as calcium or biotin, or natural ingredients such as plant extracts or phytohormones.

In some embodiments, the therapeutic agent is one or more antimalarial drugs. Exemplary antimalarial drugs include quinine, lumefantrine, chloroquine, amodiaquine, pyrimethamine, proguanil, chlorproguanil-dapsone, sulfonamides such as sulfadoxine and sulfamethoxypyridazine, mefloquine, atovaquone, primaquine, halofantrine, doxycycline, clindamycin, artemisinin and artemisinin derivatives. In some embodiments, the antimalarial drug is artemisinin or a derivative thereof. Exemplary artemisinin derivatives include artemether, dihydroartemisinin, arteether and artesunate. In some embodiments, the artemisinin derivative is artesunate.

In another embodiment, the therapeutic agent is an immunosuppressive agent. Exemplary immunosuppressive agents include glucocorticoids, cytostatics (such as alkylating agents, antimetabolites, and cytotoxic antibodies), antibodies (such as those directed against T-cell receptors or Il-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In some embodiments, the therapeutic agent is a hormone or derivative thereof. Non-limiting examples of hormones include insulin, growth hormone (e.g., human growth hormone), vasopressin, melatonin, thyroxine, thyrotropin-releasing hormone, glycoprotein hormones (e.g., luteinizing hormone, follicle-stimulating hormone, thyroid-stimulating hormone), eicosanoids, estrogen, progestin, testosterone, estradiol, cortisol, adrenaline, and other steroids.

In some embodiments, the therapeutic agent is a small molecule drug having molecular weight less than about 2500 Daltons, less than about 2000 Daltons, less than about 1500 Daltons, less than about 1000 Daltons, less than about 750 Daltons, less than about 500 Daltons, less or than about 400 Daltons. In some embodiments, the therapeutic agent is a small molecule drug having molecular weight between 200 Daltons and 400 Daltons, between 400 Daltons and 1000 Daltons, or between 500 Daltons and 2500 Daltons. In some embodiments, the therapeutic agent is selected from the group consisting of active pharmaceutical agents such as insulin, nucleic acids, peptides, bacteriophage, DNA, mRNA, human growth hormone, monoclonal antibodies, adalimumab, epinephrine, GLP-1 Receptor agonists, semaglutide, liraglutide, dulaglutide, exenatide, factor VIII, small molecule drugs, progastrin, vaccines, subunit vaccines, recombinant vaccines, polysaccharide vaccines, and conjugate vaccines, toxoid vaccines, influenza vaccine, shingles vaccine, Prevnar pneumonia vaccine, MMR vaccine, tetanus vaccine, hepatitis vaccine, HIV vaccine Ad4-env Clade C, HIV vaccine Ad4-mGag, DNA vaccines, RNA vaccines, etanercept, infliximab, filgrastim, glatiramer acetate, rituximab, bevacizumab, any molecule encapsulated in a nanoparticle, epinephrine, lysozyme, glucose-6-phosphate dehydrogenase, other enzymes, certolizumab pegol, Ustekinumab, ixekizumab, golimumab, brodalumab, guselkumab secikinumab, omalizumab, TNF-alpha inhibitors, interleukin inhibitors, vedolizumab, octreotide, teriparatide, Crispr cas9, insulin glargine, insulin detemir, insulin lispro, insulin aspart, human insulin, antisense oligonucleotides, and ondansetron.

In an exemplary embodiment, the therapeutic agent is insulin.

In some embodiments, the tissue-interfacing component described herein comprises two or more types of therapeutic agents.

In some embodiments, the therapeutic agent is present in the tissue interfacing component at a concentration such that, upon release from the tissue interfacing component, the therapeutic agent elicits a therapeutic response.

In some embodiments, the therapeutic agent may be present at a concentration below a minimal concentration generally associated with an active therapeutic agent (e.g., at a microdose concentration). For example, in some embodiments, the tissue interfacing component comprises a first therapeutic agent (e.g., a steroid) at a relatively low dose (e.g., without wishing to be bound by theory, low doses of therapeutic agents such as steroids may mediate a subject's foreign body response(s) (e.g., in response to contact by a tissue interfacing components) at a location internal to a subject). In some embodiments, the concentration of the therapeutic agent is a microdose less than or equal to 100 μg and/or 30 nMol. In other embodiments, however, the therapeutic agent is not provided in a microdose and is present in one or more amounts listed above.

Advantageously, administration of a tissue-interfacing component having a relatively high loading of API to the GI tract may permit more effective delivery of the API as compared to traditional methods. For example, without wishing to be bound by theory, delivering a drug via an injection to the GI tract has been shown to have a higher bioavailability compared to other methods.

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the self-righting article.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

### OTHER EMBODIMENTS

In some embodiments, the articles, systems, and methods disclosed comprise one or more liquid reservoirs. In some embodiments, the liquid reservoir comprises an active pharmaceutical ingredient. In some embodiments, the pharmaceutical ingredient is a liquid and/or is present within a liquid carrier (e.g., a solvent, an excipient). In some embodiments, the active pharmaceutical ingredient comprises a solid (e.g., a plurality of (nano) particles comprising the active pharmaceutical ingredient) and may be dissolved or suspended within a liquid carrier.

In some embodiments, a liquid reservoir has an internal volume of between 80 microliters and 300 microliters. In some embodiments, the liquid reservoir has an internal volume of between 80 microliters and 250 microliters. In some embodiments, a liquid reservoir has an internal volume of greater than or equal to 80 microliters, greater than or equal to 85 microliters, greater than or equal to 90 microliters, greater than or equal to 95 microliters, greater than or equal to 100 microliters, greater than or equal to 105 microliters, greater than or equal to 110 microliters, greater than or equal to 115 microliters, greater than or equal to 120 microliters, greater than or equal to 125 microliters, greater than or equal to 130 microliters, greater than or equal to 135 microliters, greater than or equal to 140 microliters, greater than or equal to 145 microliters, greater than or equal to 150 microliters, greater than or equal to 155 microliters, greater than or equal to 160 microliters, greater than or equal to 165 microliters, greater than or equal to 170 microliters, greater than or equal to 175 microliters, greater than or equal to 180 microliters, greater than or equal to 185 microliters, greater than or equal to 190 microliters, greater than or equal to 195 microliters, greater than or equal to 200 microliters, greater than or equal to 205 microliters, greater than or equal to 210 microliters, greater than or equal to 215 microliters, greater than or equal to 220 microliters, greater than or equal to 225 microliters, greater than or equal to 230 microliters, greater than or equal to 235 microliters, greater than or equal to 240 microliters, greater than or equal to 245 microliters or greater than or equal to 250 microliters. In some embodiments, a liquid reservoir has an internal volume of less than or equal to 250 microliters, less than or equal to 245 microliters, less than or equal to 240 microliters, less than or equal to 235 microliters, less than or equal to 230 microliters, less than or equal to 225 microliters, less than or equal to 220 microliters, less than or equal to 215 microliters, less than or equal to 210 microliters, less than or equal to 205 microliters, less than or equal to 200 microliters, less than or equal to 195 microliters, less than or equal to 190 microliters, less than or equal to 185 microliters, less than or equal to 180 microliters, less than or equal to 175 microliters, less than or equal to 170 microliters, less than or equal to 165 microliters, less than or equal to 160 microliters, less than or equal to 155 microliters, less than or equal to 150 microliters, less than or equal to 145 microliters, less than or equal to 140 microliters, less than or equal to 135 microliters, less than or equal to 130 microliters, less than or equal to 125 microliters, less than or equal to 120 microliters, less than or equal to 115 microliters, less than or equal to 110 microliters, less than or equal to 105 microliters, less than or equal to 100 microliters, less than or equal to 95 microliters, less than or equal to 90 microliters, less than or equal to 85 microliters or less than or equal to 80 microliters. Combinations are also possible in some embodiments. For example, in some embodiments, a liquid reservoir has an internal volume greater than or equal to 80 microliters and less than or equal to 250 microliters. Combinations of other ranges are also possible (e.g., greater than or equal to 80 microliters and less than or equal to 250 microliters. Other ranges are also possible.

As described elsewhere herein, in some embodiments, the articles, systems, and methods disclosed herein comprise a self-actuating component comprising a spring and a support material adapted to maintain the spring in at least a partially compressed state, wherein the support material is configured for at least partial degradation in a biological fluid, a reservoir comprising a liquid active pharmaceutical agent, and a tissue interfacing component operably linked to the self-actuating component, the tissue interfacing component comprising an inlet (e.g., side hole 385), an outlet (first end 302 of needle 320), and a channel fluidically connected to the inlet and the outlet. In some embodiments, a reservoir is configured to be in fluidic communication with the inlet upon activation of the self-actuating component.

In some embodiments, the self-righting article is at least partially supported by the tissue of the subject, the self-righting article orients in a direction to allow the tissue interfacing component to release at least a portion of the active pharmaceutical agent into the tissue.

In some embodiments, the self-righting article comprises a plug (e.g., septum 380) associated with the tissue interfacing component (e.g., needle 320), wherein the tissue interfacing component is configured to pierce the plug upon activation of the self-actuating component.

Those of ordinary skill in the art would understand based upon the teachings of this specification that, in some embodiments, the self-actuating component may be triggered at a desired time to actuate (e.g., upon exposure to a fluid, after a particular length of time, and/or a particular set of physiological conditions).

In some embodiments, the tissue interfacing component is dissolvable or retractable. In some embodiments, the self-righting article has an average density greater than 1 g/cm³.

In some embodiments, the support material is configured as a plug and wherein the plug is operably linked to the tissue interfacing component.

In some embodiments, the tissue interfacing component is configured as a projectile that extends substantially along a minor axis of the self-righting article.

In some embodiments the article comprises an outer shell having an exterior shape comprising a round cross-section, the outer shell defining a first axis, an actuation mechanism comprising a self-actuating component comprising a first spring component and a support material adapted to maintain the first spring component in at least a partially compressed state, wherein the support material is configured for at least partial degradation in a biological fluid, a reservoir disposed within the outer shell, the reservoir comprising a liquid active pharmaceutical agent, and a tissue interfacing component disposed relative to the outer shell and configured for transfer of liquid from the reservoir to a portion of tissue of the lumen wall at a location internal to the subject, wherein the article is self-righting having a geometric center and a center of mass, the center of mass being offset from the geometric center in a first direction along the first axis.

In some embodiments, when the article is supported by tissue of the lumen wall while being oriented such that the center of mass is offset laterally from the geometric center, the article experiences an externally applied torque due to gravity acting to orient the article with the first axis oriented along the direction of gravity such that the tissue interfacing component interacts with the tissue at the location internal to the subject, and upon at least partial degradation of the support material, the actuation mechanism acts on the liquid active pharmaceutical agent such that the tissue interfacing components releases at least a portion of the liquid active pharmaceutical agent into the tissue at the location internal to the subject.

In some embodiments, the liquid active pharmaceutical agent and the reservoir are constructed and arranged such that, when the article is oriented with the first axis oriented along the direction of gravity and the tissue interfacing component is interacting with the tissue, the center of mass of the liquid is disposed below said geometric center relative to the surface of the tissue.

In some embodiments, the liquid active pharmaceutical agent and the reservoir are constructed and arranged such that, when the article is oriented with the first axis oriented along the direction of gravity and the tissue interfacing component is interacting with the tissue, the center of mass of the liquid is disposed closer to the surface of the tissue than the geometric center of the article.

In some embodiments, the reservoir comprises a movable wall, wherein the movable wall is configured to expel liquid active pharmaceutical agent from the reservoir.

In some embodiments, the reservoir comprises a cylindrical wall, and wherein the movable wall of the reservoir comprises a plunger in slidable engagement with the cylindrical wall.

In some embodiments, the tissue interfacing component is operably linked to the self-actuating component, the tissue interfacing component comprising an inlet, an outlet, and a channel fluidically connected to the inlet and the outlet.

In some embodiments, the channel is configured to be in fluidic communication with the reservoir comprising the liquid active pharmaceutical ingredient upon activation of the activation mechanism.

In some embodiments, when the first spring component is in the first configuration, the side hole is positioned outside the reservoir comprising the liquid active pharmaceutical agent.

In some embodiments, the reservoir further comprises an end wall arranged opposite the movable wall, wherein the end wall comprises a penetrable seal, and wherein, when the first spring component assumes the first configuration, the tissue penetrating end of the injection needle is embedded in the penetrable seal, and wherein, when the first spring component assumes the second configuration, the injection needle pierces the penetrable seal.

In some embodiments, the actuation mechanism comprises a second spring component held releasably in a compressed state, the second spring component being configured to release from the compressed state, upon the first spring component moving from the first configuration into the second configuration, to drive the movable wall to expel liquid active pharmaceutical agent from the reservoir.

In some embodiments, the actuation assembly comprises a third spring component held releasably in a compressed state, the third spring component being configured to release when all or a predefined portion of expellable liquid active pharmaceutical agent from the reservoir has been expelled, the release of the third spring component causing the injection needle to retract relative to the article housing.

In some embodiments, the third spring component is arranged coaxially with the first spring component, such as radially within the first spring component, with at least partial axial overlap between the third spring component and the first spring component.

In some embodiments, the tissue interfacing component is at least partly dissolvable or becomes soft after actuation/penetration.

In some embodiments, the tissue interfacing component comprises a jet injection component and wherein the first spring component is configured to expel liquid active pharmaceutical agent through the jet injection component at a penetration velocity allowing the liquid active pharmaceutical agent to penetrate gastric submucosa of the lumen wall.

In some embodiments, the article does not comprise a solid therapeutic agent (e.g., the article comprises a liquid therapeutic agent). In some embodiments, a physiologically relevant dose may be present in a single tissue interfacing component (e.g., having a relatively high API loading).

The channel of the tissue interfacing component need not be present within the body of tissue interfacing component, but may, in some embodiments, be a channel disposed on a surface of the tissue interfacing component (e.g., configured to facilitate flow of the fluid in the fluidic reservoir to the tissue of a subject upon activation of the self-activating component). In some embodiments, the fluidic channel is disposed within the tissue interfacing component.

In some embodiments, the tissue interfacing component may comprise a first end portion configured to penetrate tissue (e.g., a tissue penetration end) and a second end portion opposite the first end portion. In some embodiments, the tissue interfacing component comprises a side wall extending between the first end portion and the second end portion.

In some embodiments, when the first spring component is in the first configuration, the side hole may be positioned outside the reservoir comprising the liquid active pharmaceutical agent. In some embodiments, the hole may be positioned adjacent the reservoir when the spring is in the second configuration.

In some embodiments, after a particular time period after activation of the self-actuating component, the tissue interfacing component may be configured to mechanically weaken, degrade, and/or dissolve. For example, at least a portion of the tissue interfacing component may, in the presence of a biological fluid, comprise a material (e.g., gelatin) that mechanically weakens, degrades, and/or dissolves.

In some embodiments, the tissue interfacing component is configured to be retracted after a particular period of time after activation of the self-actuating component. In some embodiments, the first spring component is operable from a first configuration to a second configuration up-on at least partial degradation of the support material, wherein the injection needle is retained within the article when the first spring component is in the first configuration. In some embodiments, the tissue interfacing component is configured to be advanced from the article and into tissue of the subject (e.g., such as a lumen wall). For example, movement of the spring from the first configuration to the second configuration such that the tissue interfacing component contacts and/or penetrates a tissue adjacent the article.

In some embodiments, the tissue interfacing component extends through the reservoir when the first spring assumes the first configuration and/or when the first spring assumes the second configuration.

In some embodiments, the tissue interfacing component may be retracted by actuation of the spring from the second configuration to a third configuration, such that the tissue interfacing component no longer interfaces with the tissue adjacent the article.

The distance the tissue interfacing component travels, the shape of the tissue interfacing component, the length of the tissue interfacing component, and/or the angle of a surface of the outlet of the tissue interfacing component may be adjusted to facilitate the flow of a fluid comprising an active pharmaceutical agent in accordance with the description herein.

In some embodiments, the self-actuating component may comprise a second support material configured, for example, to activate a second stage of the self-actuating component configured to interface with the fluidic reservoir. In some embodiments, the self-actuating component may facilitate the exit of the fluid (e.g., by applying pressure to the fluidic reservoir) from the fluidic reservoir into the inlet of the tissue interfacing component.

In some embodiments, the fluidic reservoir may be fluidically isolated until, for example, activation of the self-actuating component. In some embodiments, upon activation of the self-actuating component, the fluidic reservoir may be in fluidic communication with at least a portion of a tissue interfacing component (e.g., an inlet of the tissue interfacing component).

In some embodiments, the reservoir comprises a movable wall. For example, in some embodiments, the moveable wall may be configured to expel liquid active pharmaceutical agent from the reservoir. In some embodiments, the reservoir comprises a reservoir wall (e.g., having a cross-sectional shape such as a cylindrical shape, a square shape, or the like). In some embodiments, the movable wall of the reservoir comprises a plunger in slidable engagement with the reservoir wall.

In some embodiments, the reservoir comprises an end wall portion arranged opposite the movable wall. In some embodiments, the end wall comprises a penetrable seal. In some embodiments, when the first spring component assumes the first configuration, the tissue penetrating end of the tissue interfacing component is embedded in the penetrable seal. In some embodiments, when the first spring component assumes the second configuration, the tissue interfacing component pierces the penetrable seal.

In some embodiments, the tissue interfacing component comprises an injection needle.

In some embodiments, the actuation mechanism (e.g., a self-actuator as described herein) comprises a second spring component held releasably in a compressed state. In some embodiments, the second spring component is configured to release from the compressed state, upon the first spring component moving from the first configuration into the second configuration. For example, the first spring component moving from the first configuration into the second configuration may, in some embodiments, drive the movable wall to expel liquid active pharmaceutical agent from the reservoir.

In some embodiments, the actuation mechanism comprises a third spring component held releasably in a compressed state. In some embodiments, the third spring component is configured to release when all or a predefined portion of expellable liquid active pharmaceutical agent from the reservoir has been expelled. In some embodiments, the release of the third spring component causes the tissue interfacing component to retract relative to the article outer shell (e.g., such that the tissue interfacing component is contained within the article).

In some embodiments, the tissue interfacing component is at least partly dissolvable and/or becomes mechanically soft after actuation/penetration.

In some embodiments, a first actuation mechanism comprising a first spring and a first support material adapted to maintain the first spring in at least a partially compressed state, wherein the first support material is configured for at least partial degradation in a biological fluid; and a second actuation mechanism comprising a second spring and a second support material adapted to maintain the second spring in at least a partially compressed state, wherein the second support material is configured for at least partial degradation in a biological fluid.

In some embodiments, the systems further comprise a reservoir disposed within an outer shell, the reservoir comprising the drug-containing portion.

In some embodiments, the system comprises a drug-containing portion, the drug-containing portion comprising a liquid active pharmaceutical agent.

In some embodiments, a tissue interfacing component forms or comprises an injection needle operably linked to a first spring component, wherein the first spring component is operable from a first configuration to a second configuration upon at least partial degradation of a support material, wherein the injection needle is retained within the article when the first spring component is in the first configuration, and wherein the injection needle is configured to be advanced from the article and into a lumen wall by movement of the first spring component from the first configuration to the second configuration.

In some embodiments, an injection needle comprises a tissue penetration end, a second end portion opposite the tissue penetrating end, and a side wall extending between the tissue penetrating end and the second end portion, the side wall comprising a side hole disposed such that, when a first spring component is in a second configuration, fluid communication is established between a reservoir and the tissue penetration end of the injection needle.

In some embodiments, a reservoir and a tissue penetration end of an injection needle are not in fluidic communication when a first spring is in a first configuration.

In some embodiments, a reservoir further comprises an end wall arranged opposite a movable wall, wherein the end wall comprises a channel, and wherein, when a first spring component assumes a first configuration, a injection needle side hole is positioned above said channel, and wherein, when a first spring component assumes a second configuration, an injection needle side hole is positioned within said channel.

In some embodiments, the actuation mechanism comprises a second spring component held releasably in a compressed state, the second spring component being configured to release from the compressed state, upon a first spring component moving from a first configuration into a second configuration, to drive a movable wall to expel liquid active pharmaceutical agent from a reservoir into an end wall channel and into an injection needle side hole.

### EXAMPLES

The following examples are intended to illustrate some embodiments described herein, including certain aspects of the present invention, but do not exemplify the full scope of the invention.

### Example 1

Example 1 illustrates the components and assembly of exemplary system 300, as shown in FIG. 7. Exemplary components include, but are not limited to, the first spring 330 (labeled “needle spring” in FIG. 7), second spring 360 (labeled “dose spring” in FIG. 7), hub 335 (labeled “needle hub” in FIG. 7), side hole 385 in needle 320 (labeled “needle with laser cut opening” in FIG. 7), top 305 (labeled “top” in FIG. 7), tissue engaging surface 325 (labeled “bottom” in FIG. 7), plunger 365 and hub 370 (labeled “plunger” and “dose hub” in FIG. 7), and endcap 310 (labeled “endcap” in FIG. 7).

In the embodiment shown, an isomalt pellet serves as support material 340 and support material 375. Notably, support material 340 is smaller than support material 375 and is designed to dissolve before support material 375, thus triggering the first self-actuator before the second self-actuator. Dissolution of support material 340 is facilitated via contact with an external aqueous solution via opening 342 on top 305 of exemplary system 300. Similarly, dissolution of support material 375 is facilitated via contact with an external aqueous solution via an opening in the endcap (see FIG. 7).

Upon dissolution of support material 340 (e.g., isomalt), the first end of hub 335 disengages from latch 341 and the loaded (e.g., compression) applied to first spring 330 drives needle 320 into a position such that the side hole 385 of needle 320 is in fluidic communication with liquid reservoir 355 and first end 302 of needle 320 exits the device through second end 303 of septum 380 into a space exterior of the device. Similarly, upon dissolution of support material 375 (e.g., isomalt), the first end of second hub 370 disengages from latch 309 and the tension applied to second spring 360 causes the plunger 365 to move in a horizontal direction that compresses liquid drug reservoir 355 and pushes the liquid through the side hole 385 of needle 320 and into a space exterior of the device.

### Example 2

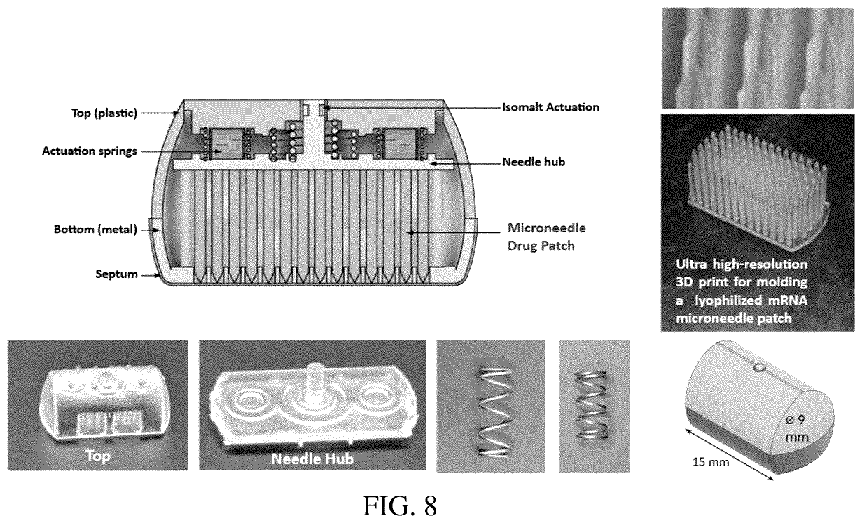

Example 2 illustrates the components and assembly of exemplary system 400, as shown in FIG. 8. Exemplary components include, but are not limited to, top 405 (labeled “Top” in FIG. 8), hub 420 (labeled “Needle Hub” in FIG. 8), first spring 430 and second spring 435 (spring adjacent “Needle Hub” in FIG. 8), third spring 440 (smaller spring adjacent CAD drawing in FIG. 8), and microarray needle 415 (labeled “microneedle patch” in FIG. 8).

In the embodiment shown, an isomalt pellet serves as support material 445. Dissolution of support material 445 is facilitated via contact with an external aqueous solution via opening 447 on top 405 of exemplary system 400. Upon dissolution of support material 445 (e.g., isomalt), the springs become disengaged, and the tension applied to the springs drives the microneedle array 415 into a second position. In some embodiments, bottom housing 410 further comprises a plurality of holes 455, the plurality of holes 455 comprising a plug (e.g., a septum) such that, upon activation of the actuator (e.g., disengagement of the latch), the microneedle array 415 pierces the plug in hole 455 into a space exterior of the device.

### Example 3

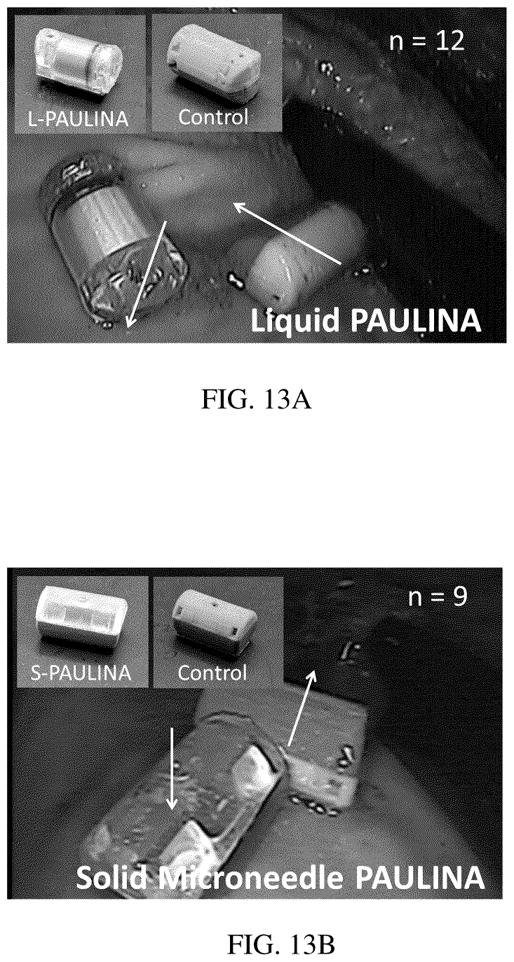

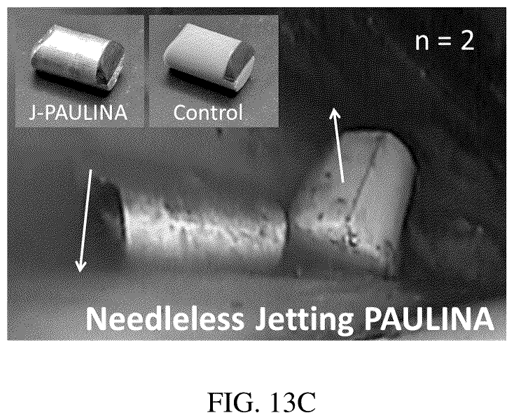

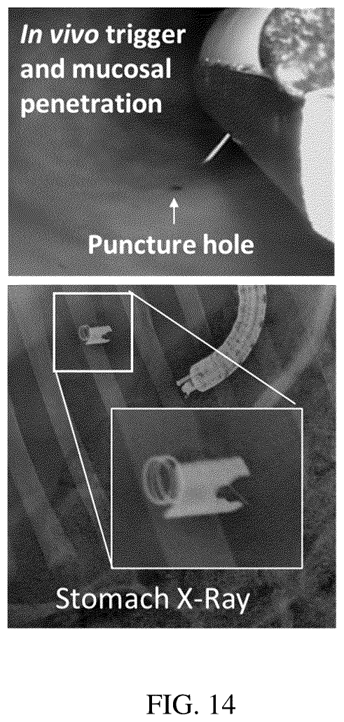

FIG. 13A shows a photomicrographs of an exemplary horizontal liquid injector (Liquid PAULINA) versus a comparative (control) device. The horizontal liquid injector is observed in a self-righted orientation relative to an in vivo surface (arrow shows direction of needle injector release), as compared to the comparative device which has not self-righted.

FIG. 13B shows a photomicrographs of an exemplary horizontal microneedle injector (Solid Microneedle PAULINA) versus a comparative (control) device. The horizontal microneedle injector is observed in a self-righted orientation relative to an in vivo surface (arrow shows direction of microneedle injection), as compared to the comparative device which has not self-righted.

FIG. 13C shows a photomicrographs of an exemplary horizontal liquid jetting injector (Jetting-PAULINA) versus a comparative (control) device. The horizontal liquid jetting injector is observed in a self-righted orientation relative to an in vivo surface (arrow shows direction of liquid release), as compared to the comparative device which has not self-righted.

FIG. 14 shows a photomicrograph of in vivo triggering of an exemplary horizontal liquid injector within the stomach of a pig. After triggering, the exposed needle penetrates the underlying tissue and delivers its liquid cargo into the tissue. The exposed needle and the resultant puncture hole are clearly visible in FIG. 14 (top). FIG. 14 also shows an x-ray of the device within the pig's stomach following deployment.

### Example 4. Simulation and Experimentation of a Jetting-Based Ingestible Therapeutic

### 4.1. Overview

To develop a device that can be used to deliver a drug in the GI tract, one of the most crucial requirements is to be within OROS. The OROS system measures 9 mm by 15 mm and provides a broad patient experience data set with respect to GI retention rates. The rate of GI retention is on the order of one in 29 million in a sample size of almost 13 billion. The OROS system has been on the market for dozens of years and is approved by the FDA for daily use. Therefore, any device that aims to be used for the administration of drugs to the GI tract requires OROS. Another requirement is the simplicity of the device. To facilitate the transition to clinical trials, passively activated devices are favorable. Self-orientation mechanisms are advantageous for delivering in the stomach, as they enable precise delivery without the need for any sensing mechanism. Lastly, adhesion of the device to the mucosa is crucial to ensure accurate delivery of the drug formulation.

Specifically, the proposed solution presents the following features engineered for human translation. First, a horizontal orientation with side delivery is adopted to maximize the drug payload to 200 μL and the internal space efficiency to 30%. The device is needle-free, can self-orient, and can passively trigger with the acid pH level present in the stomach. It respects the OROS size and uses standard compression springs in parallel to pressurize the liquid formulation and create a jet. The energy source chosen to create the jet provides less energy compared to the other methodologies used in the previously discussed work, but this ensures minimum thrust on the device and minimizes the recoil. Less recoil when jetting results in better adhesion of the device to the tissue and, therefore, a more focused jet. To have a device that works using such a low energy source, it is necessary to optimize the device and every single parameter to maximize the jet force generated at the outlet of the device. This crucial requirement is addressed by developing a robust, theory-informed simulation pipeline to guide design optimization within OROS size while ensuring device functionality and drug delivery.

In Table 1 the different ingestible devices discussed above are compared. The size of each device, the drug payload, the energy delivery method, and the presence of absence of a needle are taken into account. Lastly, it is highlighted whether a simulation approach was used to guide the device development and optimization.

Section 4.2 (Materials and Methods) of this work first presents the working principle and theory, deriving the spring-piston ODE, analytical jet-force and period expressions, and energy audit. It then lays out the simulation fundamentals, how the turbulent-flow COMSOL models driven by the ODE-based mesh motion were set up, presumed assumptions, the nozzle-shape and diameter parameter sweeps, and the full 3D device studies. Next, the prototyping methods describe both the pneumatic bench stand (pressure inlet, interchangeable cylinders/nozzles, force-sensor and high-speed imaging) the non-self-orienting devices and the final self-orienting device. Finally, the experimental protocols section details ex vivo gastric-antrum patch preparation, micro-CT depot imaging with iohexol, RNA-integrity assays, and all measurement modalities used (force transducer, high-speed video, and fluorescence).

| | | | Energy Deliver | |
|---|---|---|---|---|
| | Size | Drugload | Method | Needle |
| SOMA | OROS | 0.5 mg insulin | Springs | Drug loaded |
| | | | | dissolvable |
| | | | | microneedles |
| Robotic | [symbol] | 3.5 mg insulin | Chemical reaction | Single |
| Pill | capsule | | drive [symbol] needle | Dissolvable |
| | | | align [symbol] and | Needle |
| | | | insertion | |
| L-SOMA | ORO [symbol] | 4 mg insulin, | Springs | Single |
| | | 80 μL | | Retractable |
| | | | | Needle |
| J-SOMA | OROS | 4 mg insulin, | Pressurized CO₂ | Needle-l [symbol] ss |
| | | 80 μL | | |
| OSPRAE | [symbol] | Not available | Chemical, sodium | Needle-l [symbol] ss |
| | capsule | | bicarbonate and | |
| | | | citric acid | |
| Our | OROS | 10 mg insulin, | Springs | Needle-l [symbol] ss |
| Solution | | 200 [symbol] L | | |

[symbol]

indicates data missing or illegible when filed

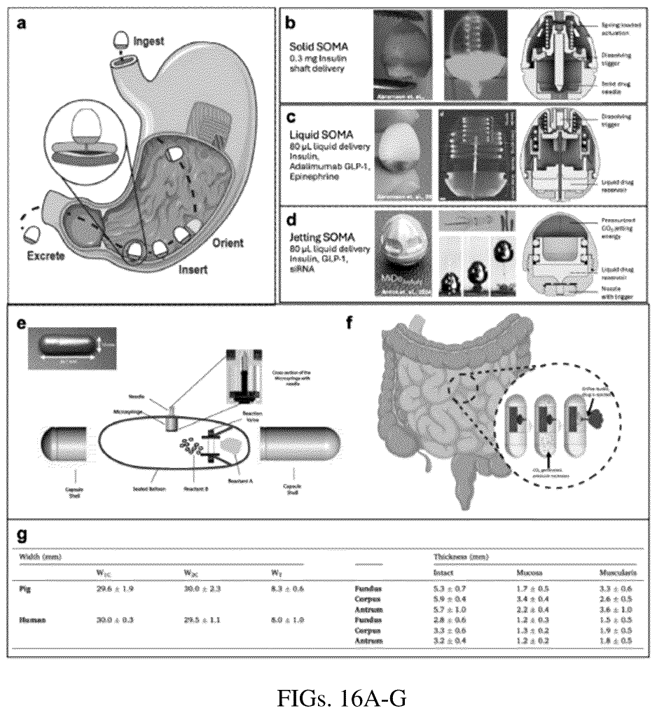

Table 1. Comparative features of ingestible drug delivery devices. Overview of the ingestible devices discussed in FIGS. 16A-G, comparing physical dimensions, drug payload capacity, energy delivery method, and presence or absence of a needle.

Section 4.3 (Results) then unfolds in three nested stages: first, Theory Validation, where the predicted piston motion of the ODE and the jetting period are compared against bench data; second, simulation outcomes, in which turbulent COMSOL runs confirm the optimal nozzle shapes, diameters, and full device force profiles; and third, experimental findings, showing how the selected cylinder/nozzle designs and spring configurations form submucosal depots (micro-CT) and preserve RNA integrity in vitro.

Section 4.4 (Discussion) integrates these three streams, theory, simulation, and experiment, into coherent design insights and outlines limitations and next steps.

Section 4.5 (Conclusion) then summarizes the key advances in self-orienting gastric jet delivery and points toward in vivo validation and broader payload applications.

### 4.2. Material and Methods

### 4.2.1. Working Principle

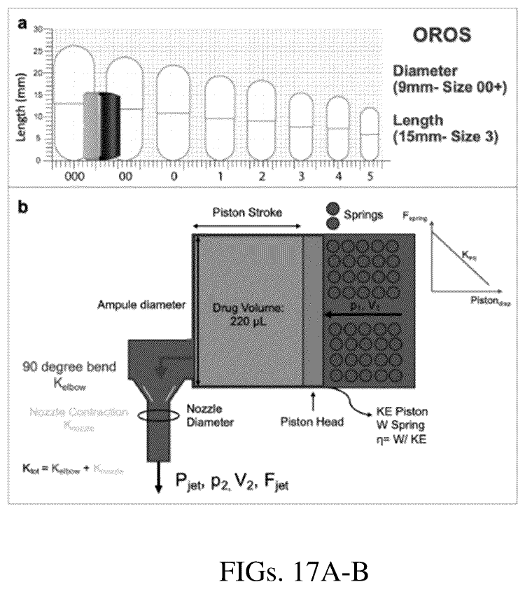

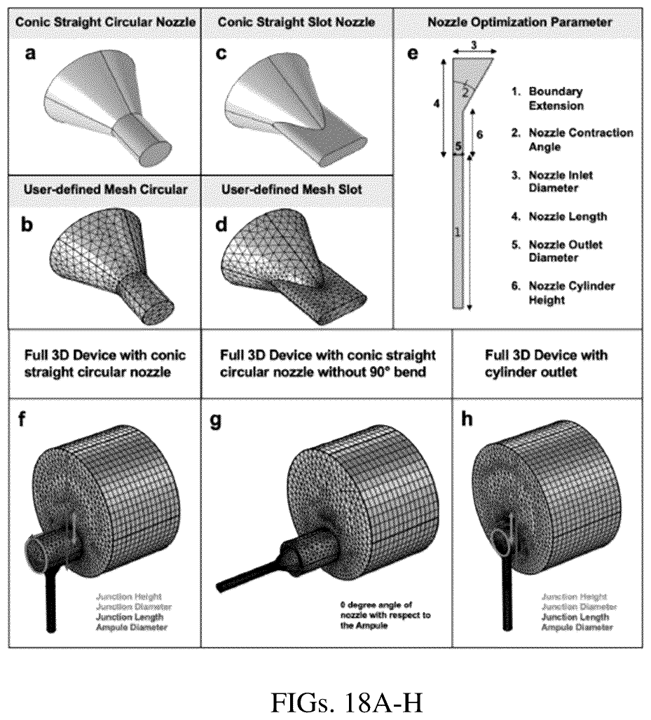

In FIGS. 17A-B the simplified schematic used to build the theoretical model is presented. The working principle is the following: the compression helical springs are placed in parallel and compressed. After triggering the piston pushes the drug through the device body into the junction and then through the 90 degree bend into the nozzle. The following components are identified from the simplified schematic: the piston head with an O-ring to ensure proper sealing is the region of the piston in direct contact with the drug formulation that is responsible of pushing it, the ampule is defined here as the area of the device body where the drug formulation is placed and stored before device triggering, the piston stroke is defined as the total distance traveled by the piston during the jetting, and the junction is referred to as the cylinder that connects the drug ampule with the nozzle. The nozzle is threaded into the junction at a 90 degree angle with respect to the device longitudinal axis, and this defines the 90 degree bend in the internal flow angle, which will be discussed later. Lastly, a cylindrical boundary extension is placed at the physical outlet of the nozzle.

The theoretical model is implemented to provide a robust fluid dynamics foundation for the simulation pipeline. The expected result of this model are the following; define the input pressure profile for different spring configurations, precisely identify the hydraulic losses introduced by the internal geometry and define the loss coefficient, derive the numerical solution of the piston displacement over time and precisely estimate the jetting period, calculate the optimal boundary extension length to minimize the numerical artifacts at the physical outlet, and lastly define the performance metric and derive and expression for it from the velocity field and the pressure field.

To model the pressure profile, the different springs were simulated in MATLAB (R2023a, The Math Works, Natick, MA, USA), after which Hooke's law was applied to define the force profile as a function of displacement. The resulting force values were then converted to pressure by dividing by the area of the piston.

$${p}_{bar} ( x ) = \frac{10}{π {( \frac{D}{2} )}^{2}} {\sum }_{i = 1}^{n} {k}_{i} max [ 0 , {L}_{i}^{0} - max ( {H}_{i}^{sol} , {C}_{i} ) - x ] \qquad ( 2.1 )$$

where:
Pbar (x): pressure in bar at piston displacement x (mm),
n: number of springs,
D: ampule diameter (mm),
ki: spring rate of spring i (N/mm),
£?: free length of spring i (mm),
H′f° 1: solid height of spring i (mm),
Ci: device-compressed height for spring i (mm),
x: piston displacement (mm).

Hydraulic losses are generally a function of changes in internal geometry, diameter contractions, nozzle effects, and internal flow bends.

To further improve the accuracy of the model and therefore of the simulation, a fine-tuning of the hydraulic losses and the jetting period estimation is performed. The Levenberg-Marquard optimization algorithm with leave-one-out cross-validation is implemented, and the following dataset is used. Measurements of the jetting period for three helical springs and four helical spring configuration were taken for 5 different outlet diameters: 450 μm, 500 μm, 600 μm, 700 μm, 800 μm. Each measurement was taken using the force sensor (Kistler 9215A piezoelectric load cell) and reading the jet period from the force profile, and three measurements were taken for each configuration. The average was then calculated, resulting in a data set of 10 data points. In Table 2 the data set is shown. The optimized hydraulic loss coefficient is recovered and the accuracy in predicting the jetting period is measured and compared to that achieved without optimization.

| | |
|---|---|
| [symbol] _{_{—}}_{3springs} | [symbol] _{_{—}}_{4springs} |

| | | | | |
|---|---|---|---|---|
| 450 μm | 32.26 | ms | 29 | ms |
| 500 μm | 26.13 | ms | 23.5 | ms |
| 600 μm | 18.15 | ms | 16 | ms |
| 700 μm | 14.6 | ms | 12.5 | ms |
| 800 μm | 10.5 | ms | 8.5 | ms |

[symbol]

indicates data missing or illegible when filed

Table 2: Dataset for Levenberg-Marquardt optimization with leave-one-out cross-validation. Dataset used for the Levenberg-Marquardt optimization algorithm with leave-one-out cross-validation. Each reported value represents the mean of three independent measurements, followed by their overall average.

### 4.2.2. Simulation Fundamentals

A simulation pipeline was developed using COMSOL MULTIPHYSrcs® (version 6.3, COMSOL AB, Stockholm, Sweden). This workflow is designed to optimize the shape and geometry of the device, while simultaneously enabling the testing of various spring configurations and nozzle systems. The primary scope of this simulation is to create a robust tool capable of accurately describing and predicting jet formation across different device configurations. This allows for a preliminary screening of designs prior to experimental testing. The key benefit of this approach lies in limiting fabrication efforts exclusively to designs that demonstrate promising simulation results, thereby significantly reducing component lead times and optimizing the number of required experiments.

### 4.2.2.1. Model Methods

To setup the simulation environment in COMSOL, the Multiphysics model is used. Within the framework of the Multiphysics simulation environment, the turbulent flow module is selected. The k-w model with the RANS-EVM turbulence model and low Re Wall Treatment is selected. Furthermore, the initial w value for turbulences model is chosen to ensure that the flow becomes turbulent only as a result of the internal flow geometry. Experimentally high-speed camera recordings will be used to determine the jet velocity at the outlet and estimate the Reynolds number at the outlet of the device. In conclusion, this turbulence model was chosen to ensure that turbulences are formed only as a result of the internal flow rather than the initial simulation setup. Different device geometries are created directly in COMSOL using the built-in function. For each design, only the fluid domain is modeled, and a No Slip Wall condition is applied along the boundaries of the 3d geometry. This strong constraint is chosen to set the velocity to zero and replicate the surface interactions between the stainless steel piston with the O-ring and the stainless-steel body and between the fluid and the stainless-steel wall of the device.

Two separate simulation models are created. A simplified model where only the nozzle is simulated to optimize its shape and the different parameters of the nozzle. In this simplified model, a linearly decaying pressure profile is applied as the boundary condition at the inlet of the nozzle. This input condition reflects only partially the real input condition; in this first stage, a relative simulation approach is preferred. The input conditions are kept fixed for every design iteration and the shape of the nozzle and the parameters are selected on the basis of the ones that achieve the highest jet power for the applied inlet condition. Here, for a faster validation process, the jet power is used as the main metric. A second more complete simulation is then created, where the whole 3d geometry is built. For this simulation a moving mesh is used as the inlet boundary condition. Specifically, the numerical solution to the piston displacement from the theoretical model is interpolated and used as the moving mesh deformation profile. A 1-ms ramp is then applied to the interpolated numerical solution to prevent exploding gradients at the start of the simulation. In addition, a mesh slip condition is applied to the lateral surface of the cylindric drug ampule to ensure proper displacement of the moving mesh at the boundary of the fluid domain. A fully developed flow boundary condition is applied to the outlet of the nozzle. This assumption will be validated in Section 4.3 with high-speed camera recordings showing the perfect columnarity of the jet at the nozzle outlet. Lastly, a Yeoh mesh smoothing type with a stiffening factor of 10 is chosen for the moving mesh deforming domain. The Yeoh model, commonly used in hyper-elastic materials, provides a nonlinear stiffening response that helps preserve mesh integrity, particularly in regions undergoing significant strain. Both simulations are performed in free air and no tissue interaction is simulated. The scope of this work is to evaluate the performance of different devices to enable the selection of the most promising devices; therefore, the main interest is to evaluate the jet formation rather than its interaction with tissue. Furthermore, the pipeline developed in this work aims to be a generalized tool for optimizing jetting-based devices for a wider spectrum of applications, while tissue interaction modeling is specific to experiments conducted within the scope of the application described in Section 4.1.

Within the simulation environment, different parameters for both the nozzle and the whole device are parameterized and then optimized running several parametric sweep. Table 3 presents the parameter space used in the simulation.

Every parameter listed will be evaluated and optimized and the chosen values will be discussed in the following sections of this work. Using the input of the theoretical model and the MATLAB script to derive the input pressure profile for different spring configurations, different spring configurations will be evaluated and tested.

| Parameter | Value | | Relevant Notes |
|---|---|---|---|
| Fluid Density | 0.996 | g/cm [symbol] | D [symbol] water mixed with methylene blue 0.01% w/v |
| Fluid Viscosity | 0. [symbol] 0 | mPa · s | D [symbol] water mixed with methylene blue 0.01% w/v |
| Turbulence Type | [symbol] | | RANS-EVM turbulence mode [symbol] Low R [symbol] Wall Treatment |
| Turbulent Kinetic Energy (k) | 1e−8 | | Fine tuned parameter based on simulation outcome |
| Specific Dissigation Rate (w) | [symbol] | | Default value |
| Nozzle Outlet Diameter | [symbol] 00 | μm | Different values tested from 450 [symbol] 800 μm |
| Nozzle Inlet Diameter | 750 | μm | Different values tested from 750 [symbol] 1000 μm |
| Nozzle Length | 1. [symbol] | mm | Fixed to minimize dead volume |
| Nozzle Contraction Angle | 40 | degrees | Different values tested from 30 [symbol] 75 degrees |
| Piston Stroke | 4 [symbol] | mm | Different drug volumes (piston strokes) were simulated |
| Boundary Extension Height | [symbol] | mm | Calculated to minimize boundary condition effects |
| Time Step | 1e−5 | [symbol] | Same time [symbol] used for the O [symbol] E [symbol] |
| M [symbol] Resolution | User Defined | | Boundary layers to model fluid w [symbol] interactions |
| Chamber Diameter | 7.5 | mm | Maximized while respecting OROS size |
| Junction Diameter | 2 | mm | Different values were tested ranging from [symbol] mm |
| Junction Length | [symbol] .5 | mm | Reduced to 0.75 mm when testing nozzleless design |

[symbol]

indicates data missing or illegible when filed

Table 3: Parameter space for nozzle and device optimization in COMSOL simulations. Geometric and operational parameters for the nozzle and full device were parameterized and optimized via multiple parametric sweeps. Listed parameters were evaluated in COMSOL MULTIPHYS1cs® (version 6.1, COMSOL AB, Stockholm, Sweden), with selected values discussed in subsequent sections.

For the definition of the mesh for both 3d geometry, the following approach is used: A user-controlled mesh type is selected and the mesh resolution is strongly increased at the 90 degree bend and at the nozzle. A swept mesh is then used to describe the drug ampule as it can be more easily resolved when applying the moving mesh slip condition. Moreover, the resolution of the different areas of the mesh was fine tuned by first starting with a more coarse mesh and then increasing the resolution until no significant differences in the solver performance could be observed.

Both the simplified simulation and the full 3d simulation use a time-dependent study with wall distance initialization. The time step used for the time-dependent simulation matches the one used for the ODE solver in the theoretical model.

### 4.2.2.2. Model Studies

The simplified simulation is first used to select between different nozzle shapes. In order to maximize penetration power, a conic straight circular nozzle is used as the first prototype. Building on top of the theoretical model where was shown that the jet force and therefore the penetration power increases with the area of the outlet a slot outlet is tested.

The two different 3d geometry are shown in FIG. 18A and in FIG. 18B with the respective user-defined mesh. The simplified model is then used to optimize the nozzle contraction angle (shown with number 2 in FIG. 18E) and the nozzle length (number 4 in FIG. 18E). The outlet diameter of the nozzle will be further optimized using the full 3d model to validate the simulation, and all the other nozzle parameters are functions of the two aforementioned. Therefore, their optimization is enough to optimize the entire nozzle. In FIG. 18E the boundary extension is also shown and its value is defined based on the value calculated in the previous section. The complete 3d simulation is used to first select the final nozzle outlet diameter (FIG. 18F), run a complete internal flow optimization, fine-tuning the junction height, the junction diameter, the junction length and the ampule diameter within the physical constraints imposed by the OROS size. Then it is used to evaluate the influence of the 90-degree bend on the device performance to understand whether its optimization is required or not. FIG. 18G shows the 3d geometry used, which perfectly replicates the previous 3d geometry with the only difference that now the nozzle is coaxial with the drug ampule. Lastly, the full 3d simulation is used to explore nozzle less design variants built in to improve drug loading while maintaining the required penetration power. The effort of this full 3d simulation effort is on the one end to maximize the peak jet force and on the other hand to maximize the drug load and the internal space efficiency (drug volume/device volume). Therefore, the expected outcome of the simulation is a device that maximizes the jet force, drug load, and internal space efficiency while respecting the OROS size constraint.

### 4.2.3. Self Orientation

the self-orientation behavior of the device was studied through a combination of geometric and inertial analysis. The device is designed to autonomously return to a stable resting configuration when perturbed, undergoing two sequential self-correcting phases governed by mass distribution, curvature geometry, and restoring torques.

To quantify these dynamics, the center of curvature (COC) (Described as the point which the curved surface of the device making contact with the external surface is concentric with) and the center of mass (COM) were first identified in 3d CAD models for two internal spring configurations (3-spring and 4-spring assemblies). The vertical distance between the COC and the COM was measured, as this offset is a key determinant of the gravitational torque. A smaller COC-COM distance generally reduces the restoring torque, while larger offsets enhance the self-righting moment. The restoring torque was estimated by calculating the gravitational torque induced by the COM offset under the assumption of small perturbations. This analysis aligns with the principles of monostable behavior, in which a body with a single stable equilibrium point always returns to that state when displaced. In this system, although the body is not geometrically homogeneous (as in the Gomboc), inhomogeneous mass distribution and geometric shaping were deliberately combined to achieve efficient self-righting.

The mechanism undergoes self-orientation in two phases: (1) an initial coarse re-alignment phase driven predominantly by asymmetric geometry and gravitational torque, followed by (2) a finer realignment phase facilitated by inertial damping and the specific curvature near the stable orientation point. The behavior is consistent with damped rolling along the physical gradient and benefits from a design that minimizes saddle-type equilibria, which would otherwise reduce efficiency.

These metrics were derived using 3d modeling and were implemented in the fabricated devices and verified through ex vivo and in vivo experiments. The aforementioned principles and restoring torque derivation were used to develop the outer geometry used in the built-in nozzle-less design where self-orientation ability is further improved.

### 4.2.4. RNA Methods

In vitro experiments were performed employing a combination of established cell-based and molecular techniques. Briefly, cells were cultured and plated under standard conditions, followed by an evaluation of cell viability. mRNA-loaded lipid nanoparticles (mRNA-LNPs) were prepared using the vortex method. The transfection efficiency was determined by a luciferase reporter assay. Finally, encapsulation efficiency was evaluated using the RiboGreen assay, and nanoparticle quality was assessed via dynamic light scattering using a Z-sizer machine.

### 4.2.5. Prototyping Methods

In this section, first the pneumatic setup is presented and then the different devices used for testing are introduced.

### 4.2.5.1. Pneumatic Setup

A pressure-regulated bench stand with interchangeable nozzle outlets is implemented. The main scope of the pneumatic setup is to have a simplified model of the jetting device to characterize the peak jet force required to create an ex vivo depot in the gastric antrum of the pig's stomach and also to better understand how to recognize and identify a depot. The setup consists of a pneumatic actuator that is directly connected to a pressurized air cylinder. The pneumatic actuator is then used to eject the loaded solution from the nozzle by pushing it through a plunger. The desired input pressure for the air cylinder can be selected using an adjustment knob. Applying the conservation of force to connected components with different surface areas, there is a conversion factor of 24 between the input pressure provided by the air cylinder and the final input pressure applied to the plunger and therefore to the liquid formulation.

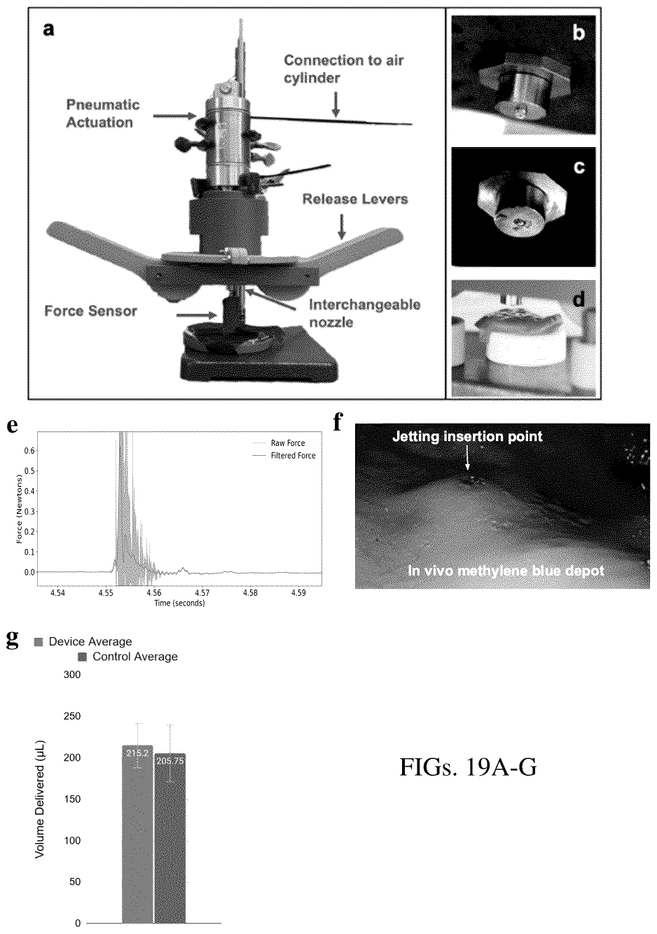

To trigger the pneumatic actuator, there are two release levers that enable an instantaneous triggering mechanism that mimics that present in the real device. In FIG. 19A the complete set-up is presented with the force sensor placed underneath the interchangeable nozzle. This provides useful information on the peak force required to achieve a depot. To establish the required pressure to form a depot, the pressure provided by the air cylinder is increased in a stepwise fashion from a value of 0.5 bar with increments of 0.1 bar to ensure a precise quantification of the total input pressure that the system of compression springs placed in parallel needs to provide. FIGS. 19B and 19C show how different nozzles can be quickly tested through pneumatic setup simply by threading different nozzles into the ampule. To test ex vivo depot formation, a 3×3 cm patch of gastric antrum from stomach tissue is placed in contact with the outlet.

### 4.2.5.2. Device

Four different devices are fabricated to enable simulation validation and experimental testing: a dummy non-self-orienting device, a dummy non-self-orienting device with a 0° internal flow angle and two different self-orienting devices, one with an external nozzle and the other with a built-in nozzle.

### Non Self Orienting Comparative Device

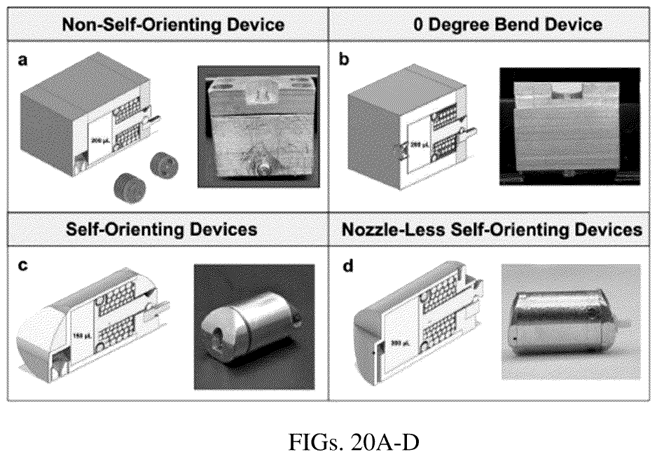

The non-self-orienting device is a simplification of the final self-orienting device (FIG. 20A). The internal geometry perfectly matches the one of the final device, but the outer geometry is adapted to simplify fabrication and ensure faster testing. This comparative device cannot self-orient and is not within the OROS size constraint. It comprises 5 main components: a stainless steel body, fabricated by an external supplier, Xometry (Xometry, Inc., Rockville, MD, USA), with a drug reservoir of 200 μl, a stainless-steel piston (Xometry, Inc., Rockville, MD, USA) with a 7.5 mm O-ring (McMaster-Carr Supply Company, Elmhurst, IL, USA), a combination of different compression helical springs depending on different device configurations (Lee Spring Company, Brooklyn, NY, USA), a brass cap fabricated in-house using a Bantam Tools Desktop CNC Milling Machine (Bantam Tools, Peekskill, NY, USA) to close the device, an external nozzle (Xometry, Inc., Rockville, MD, USA) that is threaded in the device body and a system of a stainless still pin (McMaster-Carr Supply Company, Elmhurst, IL, USA) and pellet to lock the preloaded piston. The locking mechanism uses the stainless steel pin that is placed inside the neck of the piston and then a pellet is placed inside the cap insert to keep the pin fixed inside the neck of the piston. The pellet was initially fabricated using Rigid IOK Resin on a Form 3B+ stereolithography (SLA) 3D printer (Formlabs Inc., Somerville, MA, USA) for preliminary testing. For passive triggering tests, a dissolvable pellet was produced from polyvinyl alcohol (PVA) by mold casting and subsequently processed using a MeltPrep vacuum hot-press system (MeltPrep, Graz, Austria).

The external nozzle offers greater flexibility to test different nozzles, as multiple nozzles can be tested simply by threading them into the device body.

### 0 Degree Bend Device

The same features and components apply to the dummy non-self-orienting device with a 0° internal flow angle (FIG. 20B). The assembly procedure is kept unchanged. The main difference from the non-self-orienting device lies in the nozzle position. Now, it is coaxial with the drug ampule to experimentally validate the simulation result to determine whether horizontal orientation with side delivery has a negligible effect on device performance.

### Self-Orienting Devices

The same internal geometry from the dummy non-self-orienting device is used to fabricate the self-orienting device but a few relevant differences are present. Two different versions of this device have been manufactured: one with an external nozzle and a total drug payload of 150 μl (FIG. 20C), and one with a built-in nozzle and a total drug payload of 200 μl (FIG. 20D). Both versions are within the OROS size constraint. We will refer to the former as the self-orienting device, and to the latter as the nozzle-less self-orienting device.

The devices comprise the following components: a stainless-steel body (Xometry, Inc., Rockville, MD, USA) with a built-in nozzle for the nozzle-less self-orienting device, and a threaded interface for the external nozzle in the self-orienting device; a stainless-steel piston (Xometry, Inc., Rockville, MD, USA) fitted with a 7.5 mm O-ring (McMaster-Carr Supply Company, Elmhurst, IL, USA) and various sets of springs; a cap to close the device and hold the preloaded piston in position; and the same locking mechanism described previously, consisting of a stainless steel pin (Xometry, Inc., Rockville, MD, USA) and a PVA dissolvable pellet. The cap was initially fabricated in brass in-house using a Bantam Tools Desktop CNC Milling Machine (Bantam Tools, Peekskill, NY, USA) for preliminary tolerance testing, and subsequently produced in stainless steel (Xometry, Inc., Rockville, MD, USA).

Both the two devices are assembled in the following steps: the cap is positioned with the piston and the helical springs inside the lower part of the assembly jig. The upper part of the jig is then screwed in to the lower part, compressing the helical springs until the neck piston is exposed on the outer part of the lower part of the assembly jig. The stainless steel pin is then positioned inside the neck of the piston and then the PVA pellet is placed in the cap's pocket to lock the pin is position. The cap with the preloaded piston is then removed from the assembly jig and then assembled with the body. Two dowel pins are then used to ensure full coupling between the body and the cap and prevent backfiring after device triggering. The drug formulation is then loaded using a syringe through the external nozzle or the built-in nozzle.

### 4.2.6. Experimental Protocols

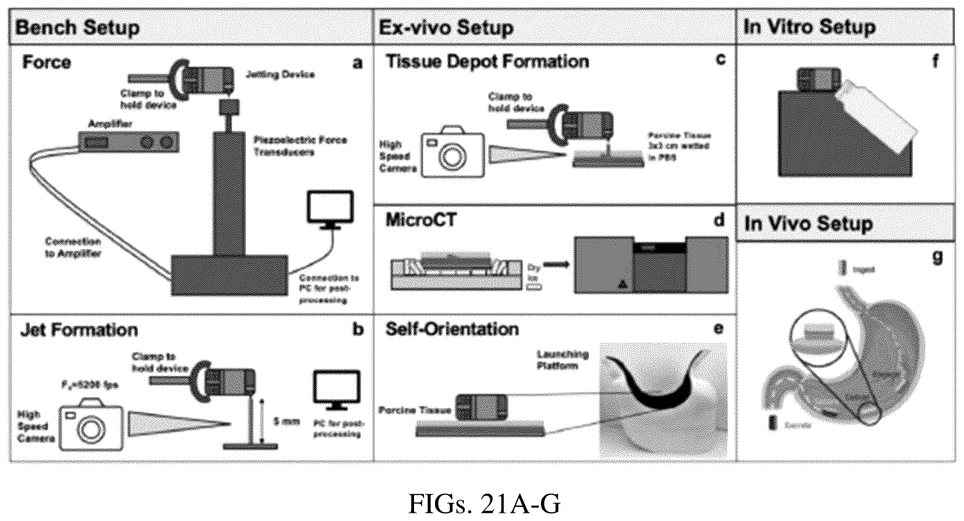

To comprehensively characterize the jet performance, delivery dynamics, and biological outcomes of the device, a series of benchtop, ex vivo, in vitro, and in vivo experiments were conducted. Each experimental modality was designed to evaluate specific functional parameters under conditions that gradually approximate physiological complexity. Bench experiments focused on quantifying jet force, velocity, and structural integrity of the jet using force sensors and high-speed imaging. Ex vivo and in vitro setups allowed tissue penetration, depot formation, and RNA delivery to be assessed, supported by imaging pipelines such as microCT. In vivo experiments evaluated the autonomous functionality of the device, including self-orientation and triggering in the gastric environment. Detailed methodologies and data acquisition protocols for each modality are outlined in the following and are presented in FIGS. 21A-G.

### 4.2.6.1. Bench Setup

To characterize the transient jetting dynamics of the device, we employed a high-sensitivity piezoelectric force transducer capable of resolving rapid, low magnitude force events. All bench measurements were performed using a Kistler 9215A piezoelectric load cell (Fz max=200 N, ø6.2 mm×23.3 mm, Kistler Group, Switzerland), specifically selected for its high signal fidelity in low-force regimes (FIG. 21A). The sensor was factory preloaded and calibrated across three force ranges: 0-2 N, 0-20 N, and 0-200 N, with a nominal sensitivity of approximately −95 pC/N. The sensor output was connected to a Kistler LabAmp Type 5165A single-channel laboratory charge amplifier (charge input range: ±100 to ±1,000,000 pC; bandwidth: 0.1 Hz to 100 kHz). The amplifier includes integrated 24-bit analog-to-digital conversion with a sampling rate of up to 200 kS/s per channel, enabling accurate acquisition of high-frequency transients. Data were sampled at 200 kHz and exported as CSV files. Post-processing was performed using a custom Python script, which applied a 4th-order low-pass Butterworth filter with a 1 kHz cutoff frequency to attenuate noise while preserving the force signal envelope. To quantify the jetting period, the filtered force trace was analyzed to identify the onset and termination of the jet impulse. The jetting window was defined as the time interval during which the filtered force exceeded 5% of the peak force amplitude. This threshold-based approach allowed for robust segmentation of the transient signal for further energy and momentum analysis. Peak detection and duration analysis were automated across datasets to maintain consistency between different nozzle and spring configurations. Matplotlib was used to visualize the date and validate the filtering, and representative traces showing raw and filtered force signals were included to illustrate the fidelity of the measurement.

High-speed imaging was used to assess the dynamics of jet formation, the columnarity of the jet, and to estimate the velocity of the jet (FIG. 21B). The recordings were made with a VEO-E 340L high-speed camera (Vision Research, USA) configured with a resolution of 896×600 pixels, a frame rate of 5,200 fps, an exposure time of 90 μs, and an exposure index of 32,000. The device was positioned 2 cm from a planar target, and the number of frames required for the jet to reach this surface was used to estimate the jet tip velocity. This information supported the calculation of an approximate Reynolds number to characterize the jetting regime.

### 4.2.6.2. Ex-Vivo Setup

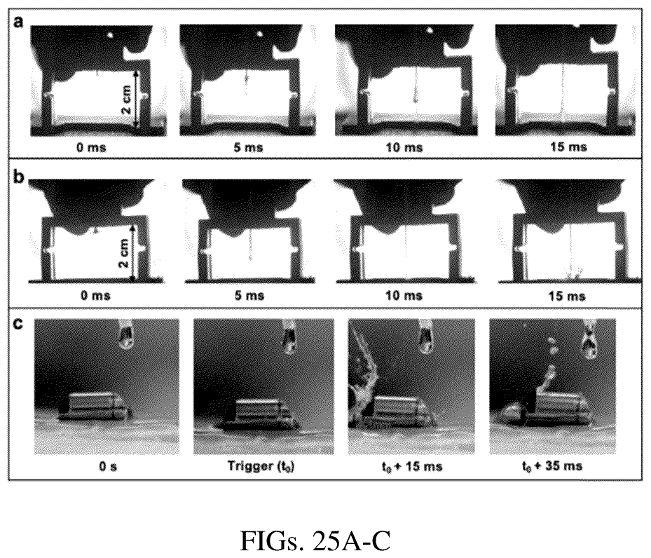

The tissue is cut from a whole pig stomach in 3×3 cm patches and classified according to the thickness of the mucosa in the different portions of the stomach. The gastric antrum is selected as the area of the stomach where the device will sit and be activated passively. To perform ex vivo depot formation studies, the tissue patch is removed from the −80° freezer and left to thaw until it reaches room temperature. The tissue is then hydrated using a solution of PBS to restore its viscoelastic properties. To perform ex vivo depot formation testing, the non-self orienting device is used. First, it is loaded with a solution of 200 μl of ultra pure water with 0.5% w/v methylene blue for visual localization in the tissue of the delivered solution. For this test, to reduce the time required for each experiment, 3D printed pellets are used and removed using tweezers. The device is positioned on the tissue using a portable jig and then triggered (FIG. 25C). The tissue in which the depot has been formed is immediately stored in the −80° freezer to prevent deterioration of the depot. After the tissue has been frozen, it is removed again from the freezer and, as it is still partially frozen, it is cut. Visual confirmation of the depot formation and precise identification of its location are performed. The high-speed camera is deployed to record the formation of the depot in the tissue and the recording is used to assess the formation of the jet, its penetration through the tissue, and the retention of dosage. These recordings will then be used to validate the final choice of nozzle outlet diameter.

MicroCT scans are then used to precisely quantify volume delivery efficiency. The device is loaded with a solution of 100% iohexol (Omnipaque oral solution, 9 mg iodine/ml). The experiment is carried out following the same procedure described before, and the tissue is flash frozen to limit the diffusion of contrast agents through the tissue and ensure an accurate depot location when the microCT scan is performed (FIG. 21D). To flash freeze the tissue, dry ice is placed in a bucket and the bucket is filled with pure Ethyl alcohol, generating a temperature of −80° C. Once the depot is formed, the tissue is dipped in the bucket and is frozen instantly. The frozen tissue is then placed in the −80° C. freezer until the CT scan is performed. To perform the scan, a SkyScan 1273 micro-CT system (Bruker microCT, Kontich, Belgium) was used. The built-in software was then used to filter CT scans and precisely calculate the volume with an accuracy of 95%.

To validate self-orientation ex vivo, a launching platform that mimics the stomach has been designed and 3d printed using Bambu Lab Xl-Carbon fused filament fabrication (FFF) 3D printer (Bambu Lab, Shenzhen, China). The stomach tissue patches are then positioned inside the 3d printed stomach to ensure realistic friction and the device is then dropped from the esophagus into the stomach. The 4 different orientations of the device are tested and each configuration is repeated 5 times and the success rate is assessed by looking at the final orientation of the device on the tissue. Video recordings are also made to ensure the two-phase orientation mechanism.

### 4.2.6.3. In Vitro Setup

Transfection experiments were conducted the day after cell plating and formulation preparation. Throughout the process, the LNP-mRNA formulations were kept on ice to minimize degradation and maintain stability. A total of seven study groups were established to evaluate the efficiency of device delivery. Before the experiment, the working surfaces, devices, and all instruments were thoroughly cleaned with 70% ethanol followed by RNaseZap (Thermo Fisher Scientific) to ensure a sterile and RNase-free environment. To preserve the integrity of the lipid nanoparticles, the entire procedure was completed in less than 30 minutes.

The self-orienting Jetting PAULINA, with N=3 technical replicates, was tested. A control group (N=1) was included, consisting of the LNP-mRNA formulation without exposure to either device. In addition, a device control replicate was included for each test. These involved briefly loading and then immediately removing the mRNA-LNP solution to assess its interaction with the device materials. Eventually, the devices were loaded with 220 μl of the LNP formulation. A slightly higher volume than the total payload was loaded to ensure the complete absence of air pockets and dead volumes. Upon activation, the devices ejected the formulations into separate sterile collection vials. For each study group, including the control, the LNP-mRNA solution was diluted in fresh complete cell culture medium to a final volume of 300 μl at a concentration of 0.0025 mg/mL. From this diluted solution, 20 μl (corresponding to 50 ng of LNPs) were added to 80 μl of medium already present in each well. Six biological replicates were prepared for each mixture to ensure statistical robustness and reproducibility.

In vitro validation of mRNA delivery was also tested using the pneumatic bench setup to ensure a smoother experimental pipeline. Input pressures larger than the one generated by the self-orienting device were used to ensure that successful results would remain valid under less stringent circumstances. Cell viability was assessed using the CellTiter-Fluor™ assay (Promega) 24 hours after treatment to ensure consistent seeding and support comparability across conditions. Transfection efficiency was quantified using the Steady-Glo@ Luciferase Assay System (Promega), measuring luciferase expression as a proxy for successful mRNA delivery. Lipid nanoparticle (LNP) characterization was performed using RiboGreen assays to determine encapsulation efficiency and dynamic light scattering using a Zetasizer (Malvern) to measure the size distribution, polydispersity index, and zeta potential.

### 4.2.6.4. In Vivo Setup

The self-orienting device is loaded with a solution of 99.5% ultrapure water and 0.5% w/v methylene blue. A pH-sensitive dissolvable polyvinylidene (PVA) pellet is used to lock the piston. The pig fasted for 24 hours prior to the experiment and was sedated at the time of the experiment. Two different tests are conducted. First, in vivo mucosal engagement and self-orientation are demonstrated. The self-orienting device, together with a control device, is dropped into the stomach through an endoscopic tube and is monitored through a camera. The control device is placed to showcase the self-orienting capability of the real device. The second test conducted is the passive activation of the device as a result of the acid pH of the pig's stomach. The device activation is assessed through the camera recording and snapshots of the pig's stomach, where the solution containing methylene blue has been delivered.

### 4.3. Results

In the next section, the results are presented. The results are organized as follows: first, the outcomes of the theoretical model are discussed, then the simulations are displayed with a focus on the final nozzle selection and the optimization of the internal flow parameters, and lastly, the experimental results are presented

### 4.3.1. Theory

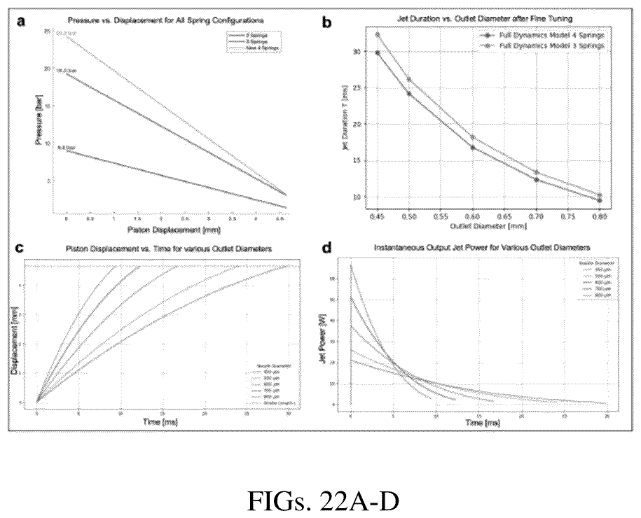

The different input pressure profiles for different spring configurations are shown in FIG. 22A. The pressure profiles are plotted against the piston displacement for the following configurations of springs: two helical springs, three helical springs, and four helical springs, all in parallel. The pressure values are calculated using an ampule diameter of 7.5 mm to maximize drug loading within the OROS size constraint, with an upper limit of 9 mm in diameter. The peak input pressure values are compared with the input pressure values retrieved from the pneumatic bench setup to ensure that the input energy is sufficient to achieve a depot. The four-spring configuration is preferable, but initial tests were conducted using a three-spring configuration to enable a first validation of the simulation results.

Using a theoretical model to describe piston displacement, we estimated the jetting period for different outlet diameters and for three- and four-spring configurations. This model involved solving a second-order ordinary differential equation (ODE) until the piston reached a full stroke. By fine-tuning the hydraulic loss parameters, the estimation error was reduced from 13.7% to 11.5%. As shown in FIG. 22B, two main trends were identified: a higher input pressure results in a shorter jetting period, and the jetting period shows a quasi-linear decay with increasing outlet diameter. This is expected as a larger nozzle outlet diameter reduces the total hydraulic resistance, leading to a higher flow rate for the same input pressure.

By solving the second-order ODE of the piston dynamic, the numerical solution of the piston displacement over time is retrieved. In FIG. 22C, the piston displacements are shown in time for different outlet diameters. The horizontal dashed line shows the stroke length after reaching which the piston is stopped by the bottom of the ampule. The plot shows that the larger the outlet diameter and the higher the velocity of the piston, as expected.

Lastly, in FIG. 22D the instantaneous output jet power is plotted for different outlet diameters. The different curves clearly show the expected trend with larger outlet diameters resulting in higher jet power. The jet power profile presents a sharp spike, the result of the sudden trigger of the piston, and then a linear decaying trend that is consistent with the linear profile of the input pressure.

Table 4 presents the results of the energy audit. Each row in the table corresponds to a different outlet diameter with its estimated jetting period.

For each different outlet diameter, the average velocity is calculated, and the maximum velocity is then derived assuming a triangular profile for the velocity of the piston. The kinetic energy for each configuration is then calculated and compared to the total work of the springs, which is always equal to 277 mJ as it does not depend on the outlet diameter but only on the springs used to drive the piston. The inertial coefficient is then calculated for each configuration taking the ratio between the kinetic energy and the total spring work. The inertial coefficient is strongly below Ie-3 for each outlet diameter, which demonstrates the validity of the linearity assumption.
Table 4: Energy audit outcomes for different nozzle outlet diameters. Estimated jetting period and calculated jet velocity were used to determine kinetic energy, which was compared with the spring work to derive the inertia coefficient.

| Do [μm] | T_jet [ms] | v_avg [m/s] | v_max [m/s] | KE [mJ] | W_spring [mJ] | η_inertia |
|---|---|---|---|---|---|---|
| 450 | 29.81 | 0.16 | 0.31 | 0.02 | 277 | 0.00007 |
| 500 | 24.15 | 0.19 | 0.39 | 0.03 | 277 | 0.0001 |
| 600 | 16.77 | 0.28 | 0.55 | 0.07 | 277 | 0.00025 |
| 700 | 12.32 | 0.38 | 0.75 | 0.12 | 277 | 0.0004 |
| 800 | 9.43 | 0.49 | 0.99 | 0.21 | 277 | 0.00075 |

### 4.3.2. Simulation

### 4.3.2.1. Nozzle Selection

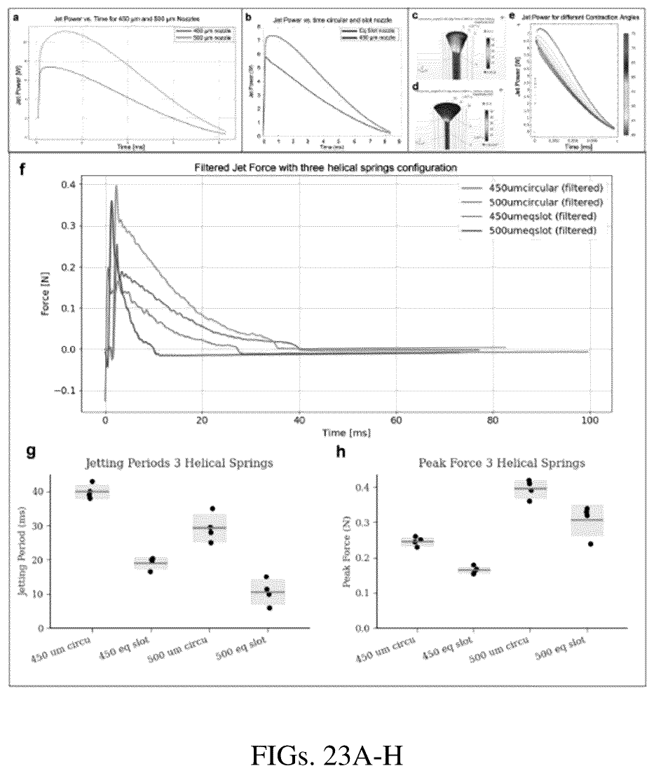

The simplified simulation model is first used to assess whether the simulation shows the same trend that was observed in the theoretical section. FIG. 23A shows that if the diameter of the nozzle outlet is increased (orange curve), the jet power increases. The simulation is then used to assess whether changing the shape of the nozzle outlet increases the penetration power. Different slot nozzles with areas equivalent to the circular nozzle are tested. The jet power generated by the 450 μm outlet diameter circular nozzle is plotted in red against the jet power generated by the equivalent slot nozzle in blue (FIG. 23B). The circular nozzle performs better than the equivalent slot nozzle, as the velocity field at the outlet of the circular nozzle is higher. The jet power shows a rapid spike at the triggering and then a linear decay as expected.

The results of the parametric sweep of the contraction angle of the conic straight cylinder nozzle are shown in FIG. 23E. A lower contraction angle produces larger jet power. The velocity field plots in FIG. 23C and FIG. 23D show how the velocity field increases for smaller contraction angles.

The force sensor experimental measurements are then used to validate the nozzle selection. In this set of experiments, a three-helical spring configuration is used. The non-self-orienting device is used and different nozzles are tested. In FIG. 23H the experimental results are shown. When comparing the performance of the circular nozzle with its equivalent slot nozzle, the circular nozzle always outperforms the slot nozzle. If the orange curve (500 um circular nozzle) is compared with the red curve (500 μm eq slot nozzle), a 0.06 N force reduction is observed. The same trend can be seen for the 450 μm circular nozzle and its equivalent slot nozzle. The jetting period for the equivalent slot nozzle is shorter than that for the circular nozzle. This finding is further discussed in Section 4.4.

Each measurement is repeated four times to ensure statistical significance. In FIG. 23G the box plot for the jetting period is reported. The mean of the four measurements is reported together with the standard deviation bar. The results show significant statistical reproducibility and a limited standard deviation. The same procedure is repeated for the peak jet force and the results are shown in 3.2.h. Statistical reproducibility is achieved across the different nozzles with only one single outlier in the peak jet force of the 500 μm eq slot.

### 4.3.2.2. Full 3D Geometry

The full 3d simulation is first validated using a parametric analysis where the nozzle circular outlet diameter is swept between the following values: 500 μm, 600 μm and 800 μm. Experimental measurements are then collected using the force sensor and the non-self-orienting device. Five replicates are taken for each outlet diameter.

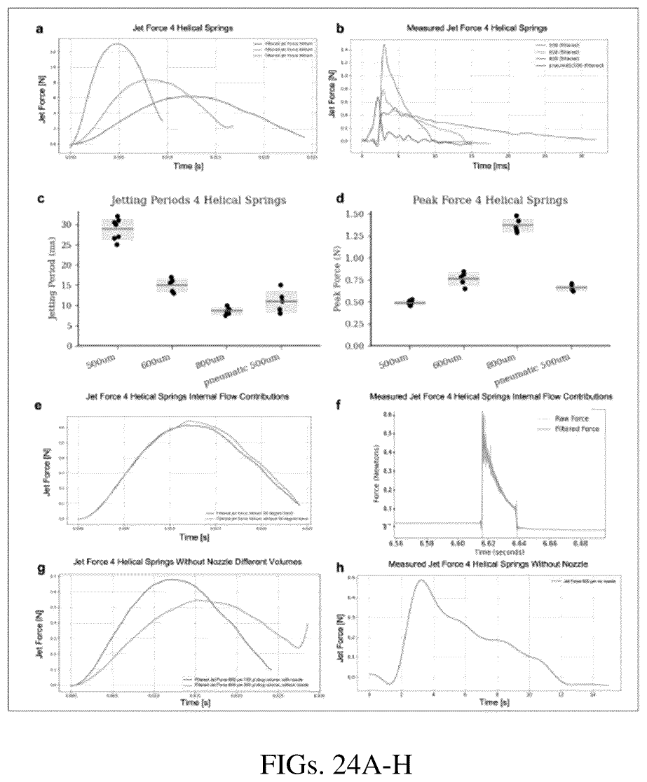

In FIG. 24A the simulation results are plotted. The peak jet force increases with the outlet diameter, and as expected, the jetting period decreases. The force profiles show a less sharp rise at the trigger and the expected linear decay after the peak. The force sensor experimental measurements are used to validate the full 3d simulation outcome. In FIG. 24B the measured filtered forces for the three different outlet diameters are plotted. The values for the peak jet force closely match the ones retrieved from the simulation. In addition, the measured jetting periods are consistent with the one in the simulation. The red curve shows the force profile for the pneumatic bench setup. This curve is the reference peak jet force required to achieve ex vivo depot formation in the gastric antrum. Each measurement is repeated five times to ensure statistical significance. In FIG. 24C the box plot for the jetting period is reported. The mean of the five measurements is reported together with the standard deviation bar. The results show significant statistical reproducibility and a limited standard deviation. The same procedure is repeated for the peak jet force and the results are shown in FIG. 24D. Statistical reproducibility is achieved across the different nozzles.

The full 3d model is leveraged to assess the effect of the horizontal orientation with side delivery on the jet force and the overall device performance. In the simulation, the same 3d geometry used to simulate the full device is used with the only difference that now the outlet is coaxial with the ampule. To validate the simulation results, the non-self-orienting dummy device without the 90 degree bend is used. In both the simulation and experimental validation, a four-helical spring configuration is used. In FIG. 24E the orange curve shows the jet force when the 90-degree internal bend is removed. The blue curve shows the jet force with the 90-degree internal bend. A slight reduction in the peak jet force is observed. In FIG. 24F the jet force measured using the non-self-orienting device with a 0-degree internal flow angle is plotted. The measured peak jet force is comparable to that shown in FIG. 24B for the 500 μm circular nozzle (blue curve).

The full 3d model is leveraged to explore built-in nozzle-less designs. The rationale behind this simulation effort is to overcome the main limitations of having an external nozzle.

Having a nozzle increases fabrication complexity, reduces internal space efficiency, limits drug payload, limits self-orientation, and challenges scalability in the assembly.

In the simulation a new 3d geometry is used where the conic nozzle is replaced with a straight cylinder outlet. FIG. 24G the results of the simulation are plotted. The blue curve shows the jet force achieved with an external nozzle while delivering a payload of 200 μl. The orange curve shows the jet force achieved with a built-in outlet while delivering a payload of 220 μl. The nozzle less design delivers a slightly lower peak jet force, but enables a higher drug load. The experimental results are shown in FIG. 24H. The measured peak jet force aligns well with the simulation. A discrepancy in the jetting period is observed with the measured jetting period being shorter than the simulated one.

### 4.3.3. Jet Parameters and Recoil

To evaluate the Reynolds number and qualitatively assess the jet columnarity, high-speed camera recordings were used. The device was placed at a distance of 2 cm from a reference plane and triggered, using a 600 μm circular nozzle. The different frames in FIG. 25A show jet formation for the three-helical spring configuration. A total of 15 ms was required for the tip of the jet to travel the fixed distance of 2 cm. The average jet velocity was therefore calculated as

$${v}_{jet} = \frac{20 mm}{15 ms} = 1.33 m / s .$$

The Reynolds number was then estimated using the following formula.

$$Re = \frac{ρ {v}_{fluid} L}{μ}$$

resulting in Re=1452 for the three-helical-spring configuration. The flow at the nozzle outlet is thus laminar, leading to stronger jet columnarity and, consequently, to a greater penetration power. This suggests that the vortices and turbulence generated within the device's internal geometry are dissipated by the time the flow exits the nozzle.

The same procedure was applied to the four-helical-spring configuration. The results are shown in FIG. 25B. After only 10 ms, the tip of the jet had already reached the reference plane, confirming that the additional spring increases the velocity of the jet and thus the penetration power. The average jet velocity in this case was vⱼₑₜ=20 mm/10 ms=2 m/s, which yields a Reynolds number of Re=2167. Once again, the flow at the outlet is laminar and strong jet columnarity is observed.

The recoil test is then performed using the self-orienting device with a circular nozzle of 600 μ m. A dissolvable PVA pellet is used to seamlessly trigger the device using water droplets. The device is placed directly in contact with the stomach tissue of the gastric antrum and the high-speed camera was used to record the triggering and assess the recoil. The snapshots in FIG. 25C illustrate the operational sequence of the device on tissue, starting with its initial placement. Subsequent frames show the piston's release, the moment of maximum recoil during the jet, which demonstrated a minimal 1 mm bounce, and finally the drug depot formed in the tissue. This minimal recoil confirms the good adhesion of the device to the tissue and its stability during operation.

### 4.3.3. Depot Formation and Imaging

### Depot Formation

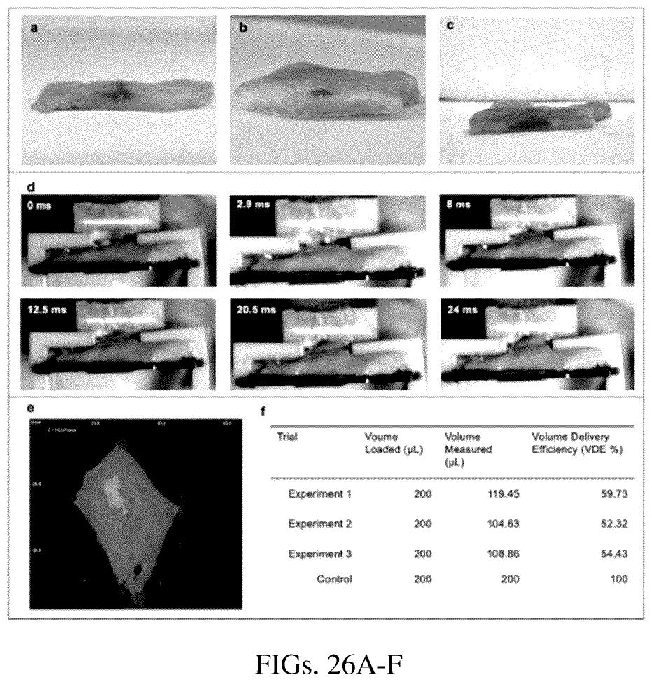

To evaluate ex vivo depot formation, porcine stomach tissue is used. The gastric antrum is specifically selected for testing as it is the region of the stomach where the device falls when swallowed. The 3×3 cm tissue patch is positioned on a foam layer to replicate the elastic deformations the stomach undergoes in vivo. The foam with the stomach on top is placed on a small tray and then slid into a jig to ensure the proper position of the device when jetting. Specifically, it is crucial to ensure a minimum offset between the device and the tissue and, at the same time, to prevent the device from physically pressing on the tissue when activating the 3D printed pellet. FIG. 26A shows the gold standard of an ex vivo depot in the gastric antrum. The pneumatic bench setup with an input pressure of 24 bar was used. The provided formulation is a 200 μ 1 solution of ultrapure water with 0.5% w/v methylene blue. In the depot, the formulation is located between the submucosal layer and the muscularis of the tissue, and this prevents it from leaking out from the edges of the tissue patch. Once the depot benchmark is created, tests are conducted using the non-self-orienting device with a 600 μm circular nozzle. In FIG. 26B, the results with a three-helical spring configuration are shown. A partial depot is formed, but the volume delivered is much lower than the one delivered in the gold standard. The tests were repeated five times in total and similar results were retrieved. The 4 helical spring configuration is then tested.

In FIG. 26C the result is shown. The formulation is fully delivered inside the tissue, the blue is located between the submucosal layer and the muscularis. The swelling generated by the depot is visible when looking at the mucosal layer of the tissue. FIG. 26D shows some frames from the high-speed camera recording of the depot formation. In the recording, the non-self-orienting device with a circular nozzle of 600 μ m diameter is used, and the final depot recovered is the one shown in FIG. 26C and discussed earlier. At 2.9 ms, the jet starts forming and, throughout the different frames, the swelling gradually increases in volume. At 24 ms, the jet is completed and a final jetting period of 24 ms is measured. This value further confirms the results discussed previously in the theoretical model and in the jet period estimation. The tissue after the test does not show any back-leakage in the formulation from the injection point, which confirms the hypothesis that a 600 μm circular nozzle is the optimal trade-off between penetration power and dosage retention.

### MicroCT Imaging

The ex vivo depot formation tests are then repeated, loading the non-self-orienting device with a 200 μl solution of 100% iohexol (Omnipaque oral solution, 9 mg iodine/ml). FIG. 26E shows first how the depot looks in the microCT scan. Volume reconstruction is shown with the thresholding applied. The volume delivery efficiency is then calculated as the ratio between the volume retrieved by the microCT software after thresholding and the volume loaded into the device. The measurements were repeated three times, and the average delivery efficiency is 56%. A control was performed by simply injecting 200 μl of the solution through a needle and a syringe. The control showed 100% VDE (volume delivery efficiency), which shows the efficacy of the built-in software to calculate the volume of the contrast agent injected.

### 4.3.5. Self Orientation

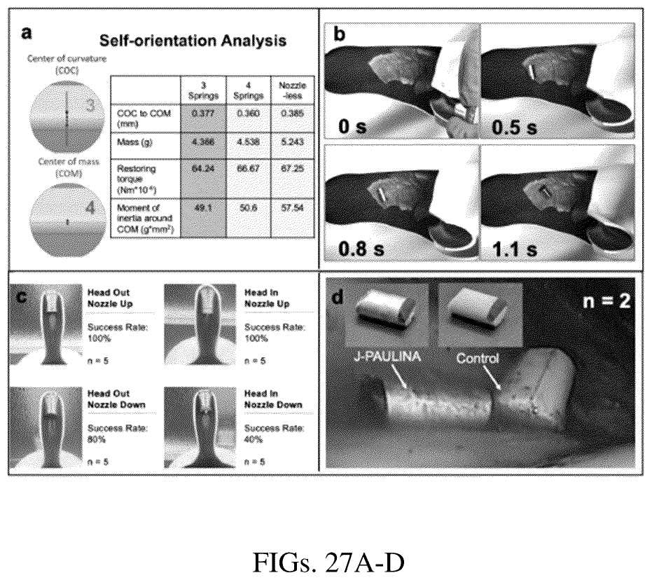

Self-orientation of the device is evaluated both ex vivo and in vivo. First, a mathematical self-orientation analysis is performed from the CAD of the different devices. In FIG. 27A the results of this analysis are shown for three helical springs configuration, the 4 helical springs configuration with an external nozzle and the 4 helical springs configuration with the built-in nozzle. To interpret the outcomes of this analysis, three parameters are crucial to achieve the device self-orientation: the distance between the center of curvature and the center of mass, the restoring torque and the moment of inertia around the center of mass. All three parameters must be maximized to ensure smoother self-orientation. For the 3 helical springs device, ex vivo tests are conducted by dropping the device into a 3d printed stomach. The stomach walls are covered with patches of stomach tissue and the device is removed from four different orientations. Each test was repeated five times. Self-orientation occurs in two separate stages.

FIG. 27B shows four frames of the ex vivo self-orientation test. At t=0 s, the device is dropped, at 0.5 s the first phase of self-orientation is completed, at t=0.8 s the second phase of self-orientation begins. After 1 s, the device is fully self-oriented and is now adhered to the stomach tissue with the nozzle facing toward it. FIG. 27C shows the success rate for each configuration. The two orientations with the nozzle facing up are favorable as they achieve a 100% success rate. The only orientation to avoid is the one with the head in and the nozzle facing down. FIG. 27D shows the results of in vivo experiments for the three-helical spring configuration. The real self-orienting device is dropped into the stomach of the pig together with a 3D-printed control device. The experiment was repeated 2 times, and the real device shows a successful self-orientation with adhesion to the mucosal layer, while the control is sitting in a casual orientation. The ex vivo and in vivo testing results were presented only for the three-helical springs configuration, that is, the one that has the lowest restoring torque and moment of inertia around COM, so a fortiori, it is conceivable that the same results will be achieved for the 4 helical springs device and the built-in nozzle less device.

### 4.3.6. In Vitro

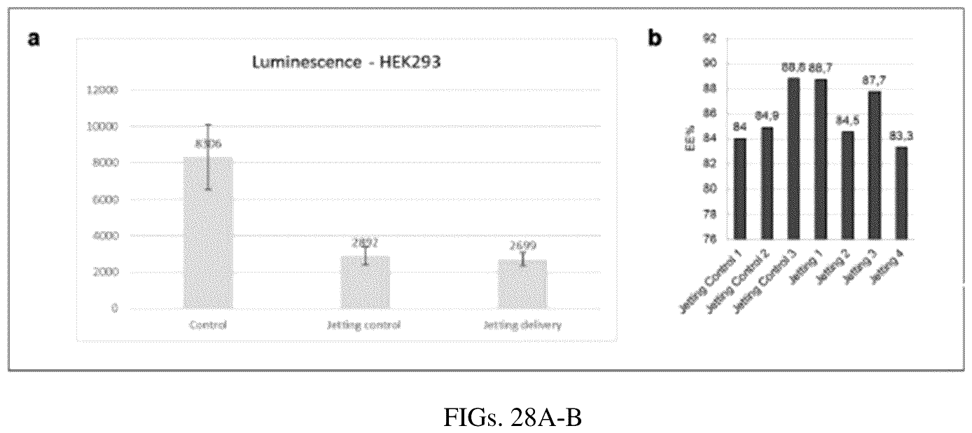

FIG. 28A presents the bioluminescence measured 24 hours after transfection. The self-orienting devices perform in a way similar to the jetting control. The jetting control is retrieved simply by loading and unloading the device without jetting. The results for the formulation not loaded into the device are plotted as a control. A significant drop is observed between the control and the jetting control. Possible explanations and mitigations are discussed in Section 4.4. FIG. 28B shows the efficiency of encapsulation for device control and for the four different replicas of the jetting. For encapsulation efficiency, similar values are measured for each different replicate and closely match the values for the jetting control. The effect of jetting forces and shear on RNA transfection efficiency is acceptable when compared to a jetting control, but the reduced performance when compared to the pure formulation requires further investigation.

### 4.4. Discussion

### 4.4.1. Results Discussions

This work aims to integrate a theoretical model with a simulation pipeline to drive device development and optimization. The first section is focused on the creation of the theoretical model aiming for a rigorous derivation, while trying to reduce, as much as possible, the simplifications introduced. The second section leverages simulations to drive device optimization, and the third focuses on simulation validation and device testing.

When defining piston dynamics, the second-order inertial effects of the piston were neglected. The validity of the linearity assumption assessed in the simulation pipeline was further confirmed through the read-out of the force sensor, justifying the adoption of a second-order ODE for the piston displacement without inertial contributions.

The values estimated for the jetting period closely match those measured in the experiments and through high-speed camera recordings. This was expected as the theoretical model was fine-tuned using the measured values of the jetting period. The measured values of the jetting period for the built-in nozzle less device do not match the simulation jetting period. The simulation is based on theoretical modeling, and the model adopts two main loss coefficients: one depending on the diameter reduction and the 90-degree bend, and the other one depending on the nozzle itself. The simulation jetting period is indeed longer than the measured one because the hydraulic losses were overestimated when modeling the piston displacement.

The experimental results closely match those of the simulation for the different circular nozzles and the slot nozzle. The slot nozzle, although it exhibits a shorter jetting period compared to the equivalent circular nozzle. This could be attributed to the diameter enlargement introduced by placing a slot instead of the circle at the outlet. The larger area results in a lower hydraulic resistance and therefore a shorter jet period, but this does not lead to a higher peak jet force because the velocity field for the slot nozzle is smaller. It is conceivable that this is due to a less uniform transition between the conical section of the nozzle and the slot itself. This is additionally demonstrated by plotting the velocity gradient, where the slot blends with the cone, the gradients are higher.

The 90-degree bend has a negligible impact on the device performance. Although the jet force is reduced compared to a device without 90 degree bend, it is still acceptable. The first reason is primarily from a fabrication point of view: optimizing the internal bend substantially increases the fabrication complexity. The second reason is due to the horizontal orientation with side delivery, one of the key features of this device. Being able to jet toward the tissue at a perfect 90-degree angle maximizes the jet efficacy and further confirms that this 90-degree internal flow bend is necessary. This is also demonstrated by the recoil test. Having a horizontal orientation and jetting at a 90-degree angle with respect to the main axis of the device strongly reduces the recoil after triggering and ensures better adhesion to the stomach, improving the overall jet delivery.

| Parameter | Value | |
|---|---|---|
| Nozzle Outlet Diameter | [symbol] | μm |
| Nozzle Inlet diameter | [symbol] 0 | μm |
| Nozzle Length | [symbol] | mm |
| Nozzle Contraction Angle | 40 | degrees |
| P [symbol] Stroke | 4. [symbol] | mm |
| Chamber Diameter | 7. [symbol] | mm |
| Junction Diameter | 2 | mm |
| Junction Length | [symbol] | mm |

[symbol]

indicates data missing or illegible when filed

Table 5: Optimized device parameters and final selected values. Overview of all device parameters optimized during development, with the final selected values used for implementation of the final device.

The theory simulation pipeline presented in this work was used to optimize every parameter of the device to maximize the jet force and provide the required penetration power with minimal input energy. In Table 5, all the optimized device parameters are presented. These values were retrieved by running several parametric simulations and were used to build the different devices that were tested and whose results have previously been presented.

The performance of the device is evaluated ex vivo by analyzing whether it can form a depot in the stomach. One of the key aspects before running the experiments is understanding the range of forces needed, depending on the nozzle used. To assess this, the pneumatic bench set-up was used, and a maximum jet force of 0.63 N was derived. The trade-off between dosage retention and penetration power is another relevant criterion when optimizing the device; otherwise, the outlet diameter would simply be maximized as much as possible. The only way to assess it is through experiments; therefore, different nozzle outlets were tested with both the pneumatic bench set-up and the non-self-orienting device. Since a nozzle with a 600 μm diameter can form a depot while allowing dosage retention, the selection of the nozzle was finalized.

MicroCT scans confirmed the location of the depot and yielded a volume delivery efficiency (VDE) of 60%. Considering that our proposed solution delivers 200 μl, which is significantly greater than the 80 μl delivered by the MiDe (AxAuto) device, this result shows promising results and warrants further testing. Volume delivery efficiency is specific to the solution used for imaging. In this case 100% iohexol (Omnipaque oral solution, 9 mg iodine/ml) was used. To mitigate for the larger viscosity of the solution, different dilutions of iohexol with ultrapure water were tested, but none of them provided a sufficient contrast factor. To assess volume delivery efficiency with a solution closer to that used in the final applications of the device, another possible mitigation is to use ultrapure water with 0.5% methylene blue and measure the weight of the tissue before and after depot formation. With a precise scale, this method could potentially provide an appropriate alternative to microCT to calculate VDE, while ensuring that the real performance of the device is evaluated.

In vitro RNA testing showed promising results when comparing the device control with jetting experiments. At the same time, the experimental procedure needs further optimization as the fluorescence results show a strong degradation when the formulation is placed inside the device. One of the potential mitigations is to thoroughly clean the device prior to testing with citric acid and sodium bicarbonate. Even a very small concentration of methylene blue left in the device from previous experiments can strongly degrade the performance of the formulation. The second main mitigation is to repeat the same experiments with the pneumatic bench set-up. Using the two-lever trigger would simplify operation, enabling a smoother test and reducing the time the temperature-sensitive formulation remains at room temperature inside the device.

Leveraging the simulation-guided approach, the presented device uses a lower energy-density energy source and optimizes every aspect behind the jet formation. Having a lower energy source combined with the horizontal orientation ensures better adhesion to the tissue, minimizing the recoil. In addition, mucosal engagement and adhesion are, in fact, two crucial requirements behind an ingestible therapeutic for drug delivery. The presented approach also enables higher internal space efficiency, resulting in a higher payload within the OROS size requirements. The nozzle less device achieves an internal space efficiency of 30% with a total drug load of 200 μl. This is made possible specifically by adopting off-the-shelf springs as the energy source. Furthermore, compression springs facilitate the assembly of the device with fewer steps required, potentially reducing the risk of device failure. On the other hand, opting for less input energy increases the risk of the device under performing and not delivering the required peak jet force if the springs become misaligned.

The porcine stomach tissue is a good proxy for the human stomach. The thickness of the gastric antrum for the human stomach is below that of the pig. Without considering stiffness and relaxation time, this corroborates that the results seen with porcine tissue can be translated to human tissue. Potentially, a spring-based system that can deliver a 200 μl formulation for a mucosal thickness less than 2 mm in the pig's stomach should be able to deliver everywhere in the human's stomach.

Generally, many of the solutions discussed show reliable performance in preliminary in vivo tests, sometimes at the cost of human translation. Jetting-based devices and, more specifically, spring-based systems have the potential to facilitate the translation of these ingestible therapeutics to humans.

### 4.4.2. Limitations and Future Directions

The work discussed so far presents a theoretical foundation that is used to develop the simulation pipeline. The simulation is then validated through experiments, and the optimized device is used to run preliminary tests. Currently, most of the experimental results have been validated ex vivo and in vitro. A more in-depth in vivo experimental effort is required to further corroborate the feasibility of this device to deliver drug formulation in vivo. Semaglutide has been selected as the optimal candidate for the first in vivo experiments with the goal of detecting its presence in the blood.

The current simulation pipeline is tuned for the specific optimization of a jetting-based drug delivery device for the stomach, but the simulation does not include any modeling of the jet tissue interaction. Implementing the jet-tissue interaction would allow the optimization of the device to be carried out while factoring in dosage retention. This would be at the cost of higher computational time, limiting the ability of this pipeline to provide real-time (24 hour) feedback to a new design.

Lastly, the current approach enables the study of the behavior of the device when loaded with different formulations by factoring in their rheological properties. This could potentially pave the way for extending this study to nonnewtonian fluids, developing a comprehensive tool to study and understand jetting principles and jetting-based devices.

### 4.5. Conclusions

This project successfully validated a comprehensive approach for the development of the next generation of jetting-based drug delivery devices. The theoretical model accurately predicted critical performance metrics, such as jet timing and force, while a COMSOL pipeline for turbulent flow reliably ranked different device configurations. Experimental testing further confirmed these findings by demonstrating effective depot delivery and maintaining payload integrity. This integrated methodology provides a powerful and efficient tool for screening and prioritizing new designs. Using the predictive capabilities of the theoretical and simulation models, we can significantly reduce the time and cost associated with physical fabrication and experimentation. With all the parameters optimized, the device is fully prepared for crucial in vivo testing.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape-such as, round, square, circular/circle, rectangular/rectangle, triangular/triangle, cylindrical/cylinder, elliptical/ellipse, (n) polygonal/(n) polygon, etc.; angular orientation-such as perpendicular, orthogonal, parallel, vertical, horizontal, collinear, etc.; contour and/or trajectory-such as, plane/planar, coplanar, hemispherical, semi-hemispherical, line/linear, hyperbolic, parabolic, flat, curved, straight, arcuate, sinusoidal, tangent/tangential, etc.; direction-such as, north, south, east, west, etc.; surface and/or bulk material properties and/or spatial/temporal resolution and/or distribution-such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

1. A self-righting article, comprising:
(i) a tissue-engaging surface;
(ii) one or more curved surfaces adjacent the tissue-engaging surface;
(iii) a drug-containing portion, wherein the drug-containing portion occupies greater than or equal to 30 vol % of the self-righting article and/or the drug-containing portion comprises greater than or equal to 250 uL of drug;
(iv) a tissue interfacing component;
(v) an actuation mechanism configured to actuate horizontally with respect to a major axis of the tissue-engaging surface; and
(vi) one or more springs, where at least one spring is operably linked to the actuation mechanism and/or the tissue interfacing component.

2-3. (canceled)

4. A self-righting article, comprising:
(i) one or more curved surfaces adjacent a tissue-engaging surface;
(ii) a drug-containing portion, wherein the drug-containing portion comprises greater than or equal to 250 uL of drug;
(iv) a liquid jet injection component;
(iv) an actuation mechanism configured to actuate horizontally with respect to a major axis of the tissue-engaging surface; and
(vi) one or more springs, where at least one spring is operably linked to the actuation mechanism and/or the jet injection component.

5. The article as in claim 1, further comprising a reservoir comprising the drug-containing portion.

6. The article as in claim 1, wherein the drug-containing portion comprises a liquid active pharmaceutical agent or a solid active pharmaceutical agent.

7. (canceled)

8. The article as in claim 1, wherein the tissue interfacing component is operably linked to the actuation mechanism.

9. The article as in claim 1, wherein when the self-righting article is oriented in a longitudinal orientation relative to an external surface:
a center of mass of the self-righting article is offset from a first axis that is perpendicular to the tissue-engaging surface, wherein the first axis passes through the center of curvature of the one or more curved surfaces, and
wherein the article rotates toward the center of mass until the tissue-engaging surface contacts the external surface.

10. A self-righting article, the self-righting article comprising:
an outer shell;
an active pharmaceutical ingredient disposed within the outer shell configured for delivery to a location internal to a subject;
the outer shell comprising:
i) a flat surface;
ii) a lateral monostable cross-section; and
iii) a longitudinal, bistable, cross-section,
such that the outer shell reorients such that the flat surface contacts a surface, regardless of an initial orientation of the self-righting article.

11. The article as in claim 1, wherein the drug-containing portion comprises a plurality of tissue interfacing components.

12. The article as in claim 1, wherein a first spring is configured to displace the tissue interfacing component and a second spring is configured to displace a liquid active pharmaceutical agent.

13. The article as in claim 1, wherein:
a first actuation mechanism comprises:
(i) a first spring and a first support material adapted to maintain the first spring in at least a partially compressed state, wherein the first support material is configured for at least partial degradation in a biological fluid; and
a second actuation mechanism comprises:
(ii) a second spring and a second support material adapted to maintain the second spring in at least a partially compressed state, wherein the second support material is configured for at least partial degradation in a biological fluid.

14-15. (canceled)

16. The article as in claim 1, wherein the tissue interfacing component forms or comprises an injection needle operably linked to a first spring component, wherein the first spring component is operable from a first configuration to a second configuration upon at least partial degradation of a support material, wherein the injection needle is retained within the article when the first spring component is in the first configuration, and wherein the injection needle is configured to be advanced from the article and into a lumen wall by movement of the first spring component from the first configuration to the second configuration.

17. The article as in claim 1, wherein an injection needle comprises a tissue penetration end, a second end portion opposite the tissue penetrating end, and a side wall extending between the tissue penetrating end and the second end portion, the side wall comprising a side hole disposed such that, when a first spring component is in a second configuration, fluid communication is established between a reservoir and the tissue penetration end of the injection needle.

18. The article as in claim 1, wherein a reservoir and a tissue penetration end of an injection needle are not in fluidic communication when a first spring is in a first configuration.

19. The article as in claim 1, wherein a reservoir further comprises an end wall arranged opposite a movable wall, wherein the end wall comprises a channel, and wherein, when a first spring component assumes a first configuration, a injection needle side hole is positioned above said channel, and wherein, when a first spring component assumes a second configuration, an injection needle side hole is positioned within said channel.

20. The article as in claim 1, wherein the actuation mechanism comprises a second spring component held releasably in a compressed state, the second spring component being configured to release from the compressed state, upon a first spring component moving from a first configuration into a second configuration, to drive a movable wall to expel liquid active pharmaceutical agent from a reservoir into an end wall channel and into an injection needle side hole.

21. The article as in claim 1, wherein the actuation mechanism comprises a self-actuating component comprising a movable microneedle hub within a reservoir, the movable microneedle hub comprising:
a plurality of spring components and a plurality of support material adapted to maintain the plurality of spring components in at least a partially compressed state, wherein the plurality of support material is configured for at least partial degradation in a biological fluid.

22. The article as in claim 1, wherein a movable microneedle hub comprises a plurality of microneedles, and is operably linked to a plurality of spring components, wherein the plurality of spring components is operable from a first configuration to a second configuration upon at least partial degradation of a support material, wherein the plurality of microneedles is retained within the article when the plurality of spring components is in the first configuration, and wherein the plurality of microneedles is configured to be advanced from the article and into a lumen wall by movement of the plurality of spring components from the first configuration to the second configuration.

23-26. (canceled)

27. The article as in claim 1, wherein when the self-righting article is oriented in a longitudinal orientation relative to an external surface:
a center of mass of the self-righting article is offset from a first axis that passes through a point of contact with an external surface and is parallel to a vector of a gravitational force, wherein the article rotates toward the center of mass until the tissue-engaging surface contacts the external surface.

28. (canceled)
